(19) United States
(12) Patent Application Publication
(10) Pub. No.: US 2026/0014130 A1
(43) Pub. Date: Jan. 15, 2026

(54) FORMULATIONS OF CAPSID INHIBITOR

(71) Applicant: Gilead Sciences, Inc., Foster City, CA (US)

(72) Inventors: Pallavi Pralhad Pawar, Fremont, CA (US); Charles W. Rowe, San Bruno, CA (US)

(21) Appl. No.: 19/244,084

(22) Filed: Jun. 20, 2025

Related U.S. Application Data
(60) Provisional application No. 63/662,961, filed on Jun. 21, 2024.
(60) Provisional application No. 63/756,195, filed on Feb. 9, 2025.

(51) Int. Cl. A61K 31/4439; A61K 47/10

(52) U.S. Cl. CPC A61K 31/4439; A61K 47/10

(57) ABSTRACT

The present disclosure relates to pharmaceutical compositions comprising an HIV capsid inhibitor and methods for the treatment or prevention of a human immunodeficiency virus (HIV) infection in a patient.

### TECHNICAL FIELD

The present disclosure relates to pharmaceutical formulations comprising a HIV capsid inhibitor and methods for the treatment or prevention of a human immunodeficiency virus (HIV) infection in a patient.

### BACKGROUND

The viral capsid protein (CA) is essential for multiple stages of the HIV life cycle. During viral maturation following the processing of Gag polyprotein by the HIV protease, CA self-assembles into the conical shaped core characteristic of mature HIV-1 virions. Contained within this capsid core are the viral RNA, nucleocapsid, reverse transcriptase, and integrase. Failure to generate a suitable core precludes infectivity. In addition, CA contributes to multiple essential processes during the early stages of HIV replication, including important roles in regulating proper capsid core disassembly (uncoating) kinetics to ensure efficient and productive viral DNA synthesis via coupled reverse transcription, and contributes to the active transport of pre-integration complexes into the nuclear compartment to support viral DNA integration into transcriptionally active loci. Defects in the proper function of capsid ultimately inhibit efficient nuclear uptake and integration of viral DNA into the host genome.

Human immunodeficiency virus type 1 infection is a life-threatening and serious disease of major public health significance, with approximately 38 million people infected worldwide and approximately 26 million on antiretroviral (ARV) treatment (UNAIDS. Global HIV & AIDS statistics, 2020 fact sheet). Advances in combination ARV therapy for HIV have led to significant improvements in morbidity and mortality by suppressing viral replication, preserving immunologic function, and averting disease progression to AIDS.

### SUMMARY

The present disclosure provides, inter alia, a pharmaceutical composition comprising a free acid form of the compound of Formula Ia:

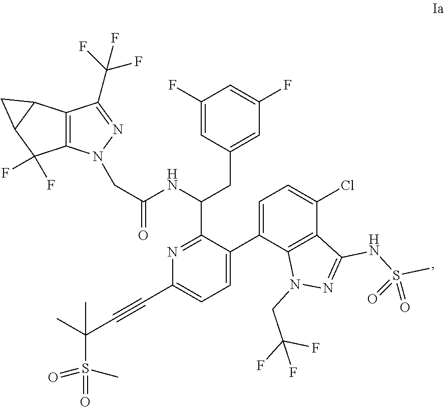 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 30% to about 45% of the free acid form of the compound of Formula Ia;
about 40% to about 50% of the PEG 300;
about 5% to about 10% of the water; and
about 7% to about 15% of the ethanol.

The present disclosure further provides a method of treating or preventing HIV in a patient, comprising administering to the patient a pharmaceutical composition provided herein.

The present disclosure further provides a pharmaceutical composition provided herein, for use in any of the methods described herein.

The present disclosure further provides use of a pharmaceutical composition provided herein, for the preparation of a medicament for use in any of the methods described herein.

### DESCRIPTION OF DRAWINGS

FIG. 1 shows lenacapavir plasma concentration over time in patients administered a one-time intramuscular dosage of Composition 2 (square trace), Composition 4 (triangle trace), or Composition 3 (circle trace). LEN refers to lenacapavir; NaS refers to lenacapavir sodium; FA refers to lenacapavir free acid; IM refers to intramuscular administration; 18G refers to 18 gauge needle for administration.

FIG. 2 shows lenacapavir plasma concentration over time in patients administered a one-time subcutaneous dosage of Composition 6 (circle trace) or Composition 3 (triangle trace). LEN refers to lenacapavir; FA refers to lenacapavir free acid; SC refers to subcutaneous administration.

FIG. 3 shows mean (90% CI) plasma concentration-time profiles following once-yearly intramuscular administration of lenacapavir 5000 mg Compositions 3 and 4. Horizontal dashed reference line at 1070.4 ng/ml represents mean Cₘₐₓ achieved at the supratherapeutic total dose of 9600 mg of oral lenacapavir in the thorough QT/QTc study. Cₘₐₓ=peak plasma concentration.

FIGS. 4A-4B show mean (90% CI) plasma concentration-time profiles of once-yearly intramuscular lenacapavir versus twice-yearly subcutaneous lenacapavir for Composition 4:5000 mg (2×5 mL 500 mg/mL with 5% w/w ethanol) (FIG. 4A); and Composition 3:5000 mg (2×5 mL 500 mg/mL with 10% w/w ethanol) (FIG. 4B). Vertical dotted line indicates week 52 interval. Horizontal dashed lines at 3.87 ng/mL represents in vitro paEC₉₅. Observed mean (90% CI) plasma concentration-time profile following administration of subcutaneous lenacapavir 927 mg on day 1 and at the end of 26 weeks, with oral lenacapavir 600 mg on days 1 and 2, in PURPOSE 1 and PURPOSE 2 studies. paEC₉₅-protein binding-adjusted 95% effective concentration.

FIG. 5 shows comparison of lenacapavir C_{trough} following once-yearly intramuscular lenacapavir 5000 mg (Compositions 4 and 3) and twice-yearly subcutaneous lenacapavir from the PURPOSE 1 and PURPOSE 2 studies. Boxes represent the interquartile range (25th and 75th percentiles), with horizontal solid lines in the middle representing the median and “whiskers” representing the 5^{th} and 95^{th} percentiles.

FIG. 6 shows a graphical representation of patient reported injection site pain.

FIGS. 7A-7B show graphical representations of patient reported impact of injection site pain on sleep.

### DETAILED DESCRIPTION

Pre-exposure prophylaxis (PrEP) strategies have been used to prevent transmission of HIV-1 infection. In locations where uptake of PrEP is high, there has been a significant population-level reduction in HIV incidence, demonstrating the potential for PrEP to contribute to population-wide HIV control (see e.g., Grulich et al, The Lancet. HIV, 2018; 5 (11): e629-e37; and Sullivan et al, Abstract LBPEC036, International AIDS Conference (IAC); July 2018, 23-27). However, for many individuals at risk for HIV, the uptake of daily oral PrEP has been a challenge. In the US, it is estimated that 1.1 million persons are at risk for HIV, yet only 240,000 are on Descovy or Truvada, and another 400,000 more started but have stopped daily oral PrEP. Several reasons likely contribute to the suboptimal uptake of PrEP, including lack of health system accessibility, medical mistrust due to experiences of homophobia, transphobia, and stigma, lack of willingness to take daily oral PrEP, concerns about side effects, and low self-perception of risk (see e.g., Center for Disease Control and Prevention, HIV/AIDS: Pre-exposure prophylaxis (PrEP); cdc.gov/hiv/basics/prep.html, 2017; and Wood et al, AIDS Behav. 2019; 23 (10): 2719-29).

Lenacapavir (LEN), a human immunodeficiency virus type 1 (HIV-1) capsid inhibitor, has the potential to provide an additional option for populations at risk of HIV acquisition. Lenacapavir can help address substantial existing PrEP barriers including 1) requirement for daily adherence, 2) stigma and concerns about disclosure and discrimination or other social harms, 3) oral medication-associated adverse events (AEs), including gastrointestinal tolerability, and 4) challenges with access to health care providers in overburdened health systems or in geographical PrEP deserts. Thus, LEN has the potential to increase the uptake of, adherence to, and thereby the scalability of PrEP, thus contributing to the overarching goal of ending the HIV-1 epidemic.

An annual administration (i.e., once every twelve months) of LEN for prevention, if efficacious, would be a potentially useful tool to help reduce HIV incidence among people who would benefit from PrEP. Accordingly, the present disclosure provides compositions comprising lenacapavir (e.g., the free acid form of lenacapavir) which are useful for dosing intervals of at least 1 year in duration, for the prevention of HIV-1 infection.

There were 1.3 million new HIV infections globally in 2023. Despite the availability of multiple pre-exposure prophylaxis (PrEP) options, only 3.5 million of the 21.2 million people who would benefit from PrEP were receiving it in 2023.

Although daily oral PrEP options are highly effective when used as directed, challenges with adherence and persistence have limited their overall impact. Longer-acting options can overcome some of the key challenges with daily oral PrEP by avoiding the requirement for adherence to daily dosing. In two phase 3 studies, twice-yearly subcutaneous lenacapavir was found to have superior efficacy to oral PrEP at preventing HIV acquisition in gender-diverse populations. A longer-acting PrEP option could further improve adherence and persistence, especially in settings with limited healthcare access.

There is increasing recognition of the important role of longer-acting antiretrovirals for HIV prevention. While daily oral PrEP options such as emtricitabine/tenofovir disoproxil fumarate (F/TDF) and emtricitabine/tenofovir alafenamide are highly effective when used as directed, challenges with adherence and persistence have greatly limited their overall impact. PrEP options with longer dosing intervals can overcome some of the key challenges with daily oral PrEP by removing the requirement for adherence to daily dosing. This potential was recently demonstrated in the phase 3 studies of twice-yearly subcutaneous lenacapavir for PrEP-PURPOSE 1 and PURPOSE 2—where lenacapavir demonstrated superior efficacy to daily oral F/TDF in a highly diverse participant population (see e.g., Bekker et al, N. Engl. J. Med. 2024; 391 (13): 1179-92; and Kelley et al, N. Engl. J. Med. 2024). Similarly, every two month intramuscular cabotegravir was demonstrated to be superior to daily oral F/TDF in two phase 3 studies (see e.g., Landovitz et al, N. Engl. J. Med. 2021; 385 (7): 595-608; and Delany-Moretlwe et al, Lancet, 2022; 399 (10337): 1779-89). A once-yearly administered lenacapavir formulation could have substantial additional benefits by further improving adherence and persistence, thereby reducing potential periods of non-protection. Furthermore, annual administration could improve PrEP access in regions with limited PrEP services and reduce the burden on care systems that may occur with regimens that require more frequent injection administration or medication dispensation.

### Pharmaceutical Compositions

Accordingly, the present disclosure provides pharmaceutical compositions comprising a compound of Formula Ia or Ib:

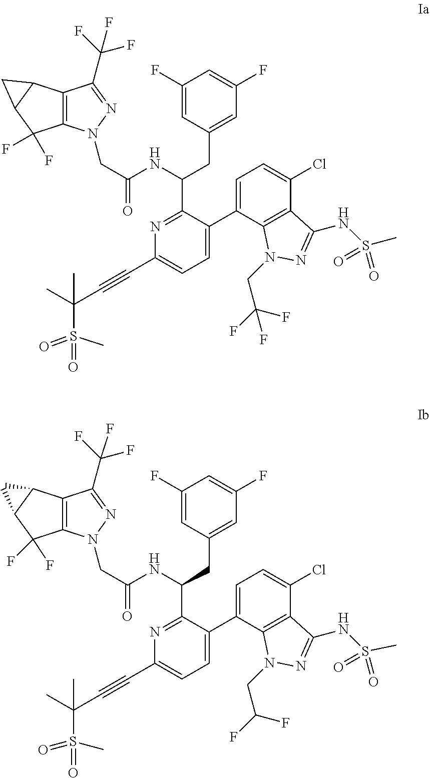 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O ; CC(C)(C#Cc1ccc(-c2ccc(Cl)c3c(NS(C)(=O)=O)nn(CC(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, and at least one pharmaceutically acceptable carrier. In some embodiments, the present disclosure provides pharmaceutical compositions comprising a free acid form of the compound of Formula Ia or Ib, and at least one pharmaceutically acceptable carrier.

In some embodiments, the free acid form of the compound of Formula Ia or Ib is prepared according to a process disclosed herein. In some embodiments, the free acid form of the compound of Formula Ia or Ib is prepared according to a process comprising reacting a sodium salt of the compound of Formula Ia or Ib with an acid in the presence of a solvent.

In some embodiments, the acid is a weak acid. In some embodiments, the acid is a strong acid. In some embodiments, the acid is an organic acid. In some embodiments, the organic acid is a weak organic acid. In some embodiments, the organic acid is a strong organic acid.

In some embodiments, the acid is selected from acetic acid, sulfuric acid, oxalic acid, citric acid, phosphoric acid, chloroacetic acid, nitric acid, formic acid, lactic acid, ascorbic acid, benzoic acid, and propionic acid. In some embodiments, the acid is acetic acid. In some embodiments, the acid is glacial acetic acid.

In some embodiments, the solvent is an organic solvent. In some embodiments, the solvent is a polar organic solvent. In some embodiments, the solvent is selected from an alcohol (e.g. methanol, ethanol, propanol, isopropanol, butanol), an ether (e.g., diethyl ether, 1,4-dioxane, tetrahydrofuran, 2-methyltetrahydrofuran), a hydrocarbon solvent (e.g. n-hexane, n-heptane, toluene, xylenes), an ester (e.g. methyl acetate, ethyl acetate, isopropyl acetate, isobutyl acetate), dichloromethane, acetonitrile, a ketone (e.g. acetone, methyl ethyl ketone, methyl isobutyl ketone), or any mixture thereof, optionally in combination with water.

In some embodiments, the solvent is selected from methanol, ethanol, propanol, isopropanol, butanol, diethyl ether, 1,4-dioxane, tetrahydrofuran, 2-methyltetrahydrofuran, n-hexane, n-heptane, toluene, xylenes, methyl acetate, ethyl acetate, isopropyl acetate, isobutyl acetate, dichloromethane, acetonitrile, acetone, methyl ethyl ketone, methyl isobutyl ketone, or any mixture thereof, optionally in combination with water.

In some embodiments, the solvent is selected from methanol, ethanol, propanol, isopropanol, and butanol. In some embodiments, the solvent is ethanol. In some embodiments, the solvent is anhydrous ethanol.

In some embodiments, the reacting is performed at a temperature of from about 10° C. to about 100° C. In some embodiments, the reacting is performed at a temperature of from about 10° C. to about 30° C. In some embodiments, the reacting is performed at a temperature of from about 15° C. to about 25° C. In some embodiments, the reacting is performed at a temperature of from about 19° C. to about 25° C. In some embodiments, the reacting is performed at a temperature of about 22° C.

In some embodiments, the free acid form of the compound of Formula Ia or Ib is prepared according to a process comprising reacting a sodium salt of the compound of Formula Ia or Ib with glacial acetic acid in the presence of anhydrous ethanol.

In some embodiments, the free acid form of the compound of Formula Ia is prepared according to a process comprising reacting a sodium salt of the compound of Formula Ia with glacial acetic acid in the presence of anhydrous ethanol.

In some embodiments, the free acid form of the compound of Formula Ib is prepared according to a process comprising reacting a sodium salt of the compound of Formula Ib with glacial acetic acid in the presence of anhydrous ethanol.

Pharmaceutical compositions of the disclosure are formulated so as to allow the active ingredients contained therein to be bioavailable upon administration of the composition to a patient. Compositions that will be administered to a patient take the form of one or more dosage units, where for example, a container of the compound of Formula Ia or Ib, or a pharmaceutically acceptable salt or free acid form thereof, in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the compound of Formula Ia or Ib, or a pharmaceutically acceptable salt or free acid form thereof, for treating an HIV infection, prevention of an HIV infection, or reducing the risk of acquiring HIV, as described herein.

As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, the compound of Formula Ia or Ib is administered as a salt, such as a pharmaceutically acceptable salt. A salt generally refers to a derivative of a disclosed compound wherein the parent compound is modified by converting an existing acid or base moiety to its salt form. A pharmaceutically acceptable salt is one that, within the scope of sound medical judgment, is suitable for use in contact with the tissues of human beings and animals without excessive toxicity, irritation, allergic response, or other problem or complication, commensurate with a reasonable benefit/risk ratio. Examples of pharmaceutically acceptable salts include, but are not limited to, mineral or organic acid salts of basic residues such as amines, alkali or organic salts of acidic residues such as carboxylic acids, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

Reference to “about” a value or parameter herein includes (and describes) embodiments that are directed to that value or parameter per se. In certain embodiments, the term “about” may include the indicated amount ±10%. In other embodiments, the term “about” may include the indicated amount ±5%. In certain other embodiments, the term “about” may include the indicated amount ±1%. Also, the term “about X” includes description of “X”.

In some embodiments, the salt is a sodium salt. In some embodiments, the pharmaceutically acceptable salt of the compound of Formula Ia provided herein is a sodium salt. In some embodiments, the pharmaceutically acceptable salt of the compound of Formula Ib provided herein is a sodium salt.

In certain embodiments, the active ingredient (for example, a compound of Formula Ia or Ib) of a composition provided herein is present as a sodium salt. In certain embodiments, the active ingredient (for example, a compound of Formula Ia or Ib) of a composition provided herein is present as a free acid.

In some embodiments, the active ingredient is a compound of Formula Ia. In some embodiments, the active ingredient is a sodium salt of the compound of Formula Ia. In some embodiments, the active ingredient is a free acid form of the compound of Formula Ia.

In some embodiments, the active ingredient is a compound of Formula Ib. In some embodiments, the active ingredient is a sodium salt of the compound of Formula Ib. In some embodiments, the active ingredient is a free acid form of the compound of Formula Ib.

In some embodiments, the present disclosure relates to a pharmaceutical composition comprising the compound of Formula Ia:

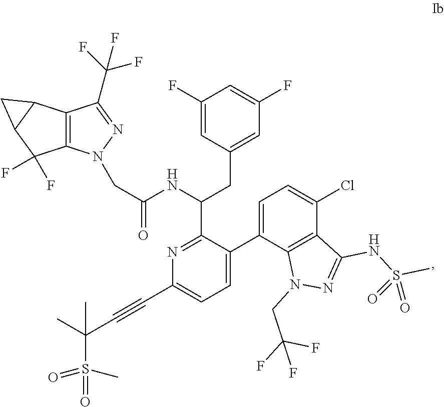 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, PEG 300, water, and ethanol.

In some embodiments, the present disclosure relates to a pharmaceutical composition comprising the compound of Formula Ib:

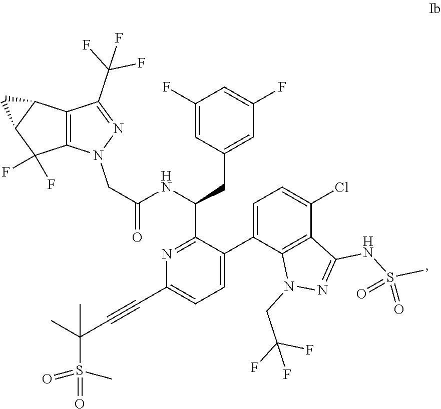 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, PEG 300, water, and ethanol.

In some embodiments, the present disclosure relates to a pharmaceutical composition comprising the compound of Formula Ia:

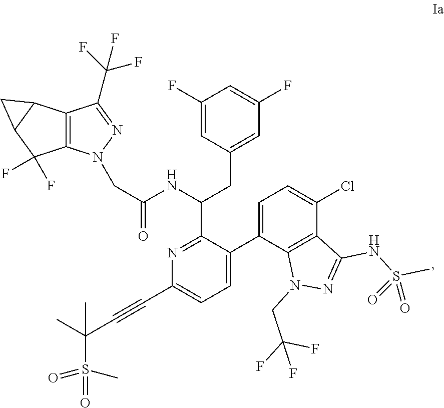 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, PEG 300, poloxamer 188, and water.

In some embodiments, the present disclosure relates to a pharmaceutical composition comprising the compound of Formula Ib:

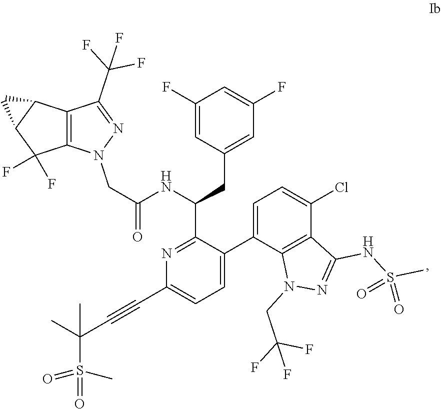 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, PEG 300, poloxamer 188, and water.

As used herein, polyethylene glycol (“PEG”) refers to a polyether having a general formula H—(O—CH₂—CH₂)ₙ—OH. In certain embodiments, the PEG may be “capped” by an alkyl group. In those embodiments, the capped PEG is of the formula alkyl-(O—CH₂—CH₂)ₙ—O-alkyl (for example, CH₃—(O—CH₂—CH₂)ₙ—OCH₃). The pharmaceutical compositions of the present disclosure can include PEG having an average molecular weight of about 100 to about 1000. In some embodiments, the average molecular weight of PEG within the pharmaceutical composition is about 100 to about 800. In some embodiments, the average molecular weight of PEG within the pharmaceutical composition is about 200 to about 600. In some embodiments, the average molecular weight of PEG within the pharmaceutical composition is about 400. In some embodiments, the average molecular weight of PEG within the pharmaceutical composition is about 300. In some embodiments, the average molecular weight of PEG within the pharmaceutical composition is about 200. In some embodiments of the pharmaceutical composition, different molecular weight PEG can be combined to obtain a desired property or properties (for example, viscosity). Specific examples of PEG include, but are not limited to, PEG 100, PEG 200, PEG 300, PEG 400, PEG 500, and PEG 600. PEG 100, for example, refers to a polyethylene glycol with an average molecular weight of about 100.

In general, poloxamers are synthetic non-ionic triblock of linear copolymers having a central hydrophobic chain of polyoxypropylene adjacent to two hydrophilic polypropylene oxide, in certain instances in a 4:2:4 weight ratio. Accordingly, in certain embodiments, the compositions disclosed herein include a compound of Formula Ia or Ib, or a pharmaceutically acceptable salt thereof, and a block copolymer comprised of one polyoxypropylene segment and two hydrophilic polypropylene oxide segments. In certain embodiments, the ratio of the polyoxypropylene segment to the two hydrophilic polypropylene oxide segments is 4:2:4 (hydrophilic polypropylene oxide: polyoxypropylene: hydrophilic polypropylene oxide). Poloxamers are generally understood to have the following structure:

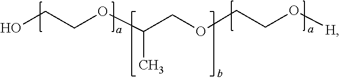

here a and b are integers. For example, a is between about 2 and about 130 and b is between about 15 and about 67. Poloxamer 188, for example, is understood to have a molecular weight from about 7680 to about 9510 Daltons (where a is about 80 and b is about 27) (see, for example, International Journal of Pharm Tech Research, Vol. 1, No. 2, pp 299-303, April-June 2009). In some instances, poloxamer 188 has an average molecular weight of about 8400 Daltons.

In some embodiments, the present disclosure relates to a pharmaceutical composition comprising a free acid form of the compound of Formula Ia:

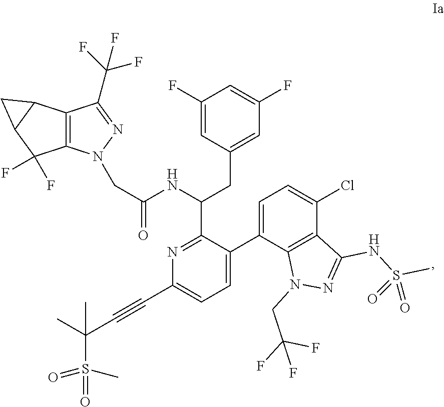 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol.

In some embodiments, the present disclosure relates to a pharmaceutical composition comprising a free acid form of the compound of Formula Ib:

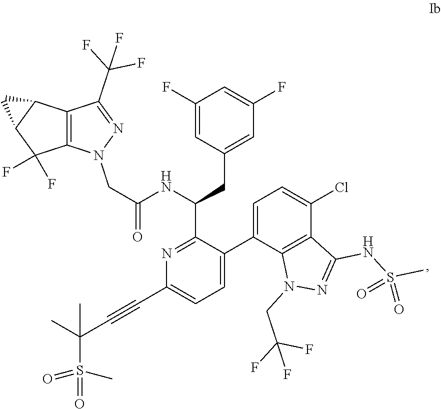 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol.

Any of the compositions disclosed herein may consist essentially of the specified ingredients. Any of the compositions disclosed herein may consist of the specified ingredients, i.e. they may contain only the specified ingredients.

As used herein, the % of an ingredient in the composition refers to the percentage on a weight basis, i.e. w/w %.

In some embodiments, the composition comprises about 30% to about 45% of the compound of Formula Ia or Formula Ib, or a pharmaceutically acceptable salt thereof, for example, about 30.0%, about 30.1%, about 30.2%, about 30.3%, about 30.4%, about 30.5%, about 30.6%, about 30.7%, about 30.8%, about 30.9%, about 31.0%, about 31.1%, about 31.2%, about 31.3%, about 31.4%, about 31.5%, about 31.6%, about 31.7%, about 31.8%, about 31.9%, about 32.0%, about 32.1%, about 32.2%, about 32.3%, about 32.4%, about 32.5%, about 32.6%, about 32.7%, about 32.8%, about 32.9%, about 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, about 35.0%, about 35.1%, about 35.2%, about 35.3%, about 35.4%, about 35.5%, about 35.6%, about 35.7%, about 35.8%, about 35.9%, about 36.0%, about 36.1%, about 36.2%, about 36.3%, about 36.4%, about 36.5%, about 36.6%, about 36.7%, about 36.8%, about 36.9%, about 37.0%, about 37.1%, about 37.2%, about 37.3%, about 37.4%, about 37.5%, about 37.6%, about 37.7%, about 37.8%, about 37.9%, about 38.0%, about 38.1%, about 38.2%, about 38.3%, about 38.4%, about 38.5%, about 38.6%, about 38.7%, about 38.8%, about 38.9%, about 39.0%, about 39.1%, about 39.2%, about 39.3%, about 39.4%, about 39.5%, about 39.6%, about 39.7%, about 39.8%, about 39.9%, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, about 43.0%, about 43.1%, about 43.2%, about 43.3%, about 43.4%, about 43.5%, about 43.6%, about 43.7%, about 43.8%, about 43.9%, about 44.0%, about 44.1%, about 44.2%, about 44.3%, about 44.4%, about 44.5%, about 44.6%, about 44.7%, about 44.8%, about 44.9%, or about 45.0% of the compound of Formula Ia or Formula Ib, or a pharmaceutically acceptable salt thereof.

In some embodiments, the composition comprises about 30% to about 45% of the free acid form of the compound of Formula Ia or Formula Ib, for example, about 30.0%, about 30.1%, about 30.2%, about 30.3%, about 30.4%, about 30.5%, about 30.6%, about 30.7%, about 30.8%, about 30.9%, about 31.0%, about 31.1%, about 31.2%, about 31.3%, about 31.4%, about 31.5%, about 31.6%, about 31.7%, about 31.8%, about 31.9%, about 32.0%, about 32.1%, about 32.2%, about 32.3%, about 32.4%, about 32.5%, about 32.6%, about 32.7%, about 32.8%, about 32.9%, about 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, about 35.0%, about 35.1%, about 35.2%, about 35.3%, about 35.4%, about 35.5%, about 35.6%, about 35.7%, about 35.8%, about 35.9%, about 36.0%, about 36.1%, about 36.2%, about 36.3%, about 36.4%, about 36.5%, about 36.6%, about 36.7%, about 36.8%, about 36.9%, about 37.0%, about 37.1%, about 37.2%, about 37.3%, about 37.4%, about 37.5%, about 37.6%, about 37.7%, about 37.8%, about 37.9%, about 38.0%, about 38.1%, about 38.2%, about 38.3%, about 38.4%, about 38.5%, about 38.6%, about 38.7%, about 38.8%, about 38.9%, about 39.0%, about 39.1%, about 39.2%, about 39.3%, about 39.4%, about 39.5%, about 39.6%, about 39.7%, about 39.8%, about 39.9%, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, about 43.0%, about 43.1%, about 43.2%, about 43.3%, about 43.4%, about 43.5%, about 43.6%, about 43.7%, about 43.8%, about 43.9%, about 44.0%, about 44.1%, about 44.2%, about 44.3%, about 44.4%, about 44.5%, about 44.6%, about 44.7%, about 44.8%, about 44.9%, or about 45.0% of the free acid form of the compound of Formula Ia or Formula Ib.

In some embodiments, the composition comprises about 35% to about 55% of the PEG 300, for example, about 35.0%, about 35.1%, about 35.2%, about 35.3%, about 35.4%, about 35.5%, about 35.6%, about 35.7%, about 35.8%, about 35.9%, about 36.0%, about 36.1%, about 36.2%, about 36.3%, about 36.4%, about 36.5%, about 36.6%, about 36.7%, about 36.8%, about 36.9%, about 37.0%, about 37.1%, about 37.2%, about 37.3%, about 37.4%, about 37.5%, about 37.6%, about 37.7%, about 37.8%, about 37.9%, about 38.0%, about 38.1%, about 38.2%, about 38.3%, about 38.4%, about 38.5%, about 38.6%, about 38.7%, about 38.8%, about 38.9%, about 39.0%, about 39.1%, about 39.2%, about 39.3%, about 39.4%, about 39.5%, about 39.6%, about 39.7%, about 39.8%, about 39.9%, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, about 43.0%, about 43.1%, about 43.2%, about 43.3%, about 43.4%, about 43.5%, about 43.6%, about 43.7%, about 43.8%, about 43.9%, about 44.0%, about 44.1%, about 44.2%, about 44.3%, about 44.4%, about 44.5%, about 44.6%, about 44.7%, about 44.8%, about 44.9%, about 45.0%, about 45.1%, about 45.2%, about 45.3%, about 45.4%, about 45.5%, about 45.6%, about 45.7%, about 45.8%, about 45.9%, about 46.0%, about 46.1%, about 46.2%, about 46.3%, about 46.4%, about 46.5%, about 46.6%, about 46.7%, about 46.8%, about 46.9%, about 47.0%, about 47.1%, about 47.2%, about 47.3%, about 47.4%, about 47.5%, about 47.6%, about 47.7%, about 47.8%, about 47.9%, about 48.0%, about 48.1%, about 48.2%, about 48.3%, about 48.4%, about 48.5%, about 48.6%, about 48.7%, about 48.8%, about 48.9%, about 49.0%, about 49.1%, about 49.2%, about 49.3%, about 49.4%, about 49.5%, about 49.6%, about 49.7%, about 49.8%, about 49.9%, about 50.0%, about 50.1%, about 50.2%, about 50.3%, about 50.4%, about 50.5%, about 50.6%, about 50.7%, about 50.8%, about 50.9%, about 51.0%, about 51.1%, about 51.2%, about 51.3%, about 51.4%, about 51.5%, about 51.6%, about 51.7%, about 51.8%, about 51.9%, about 52.0%, about 52.1%, about 52.2%, about 52.3%, about 52.4%, about 52.5%, about 52.6%, about 52.7%, about 52.8%, about 52.9%, about 53.0%, about 53.1%, about 53.2%, about 53.3%, about 53.4%, about 53.5%, about 53.6%, about 53.7%, about 53.8%, about 53.9%, about 54.0%, about 54.1%, about 54.2%, about 54.3%, about 54.4%, about 54.5%, about 54.6%, about 54.7%, about 54.8%, about 54.9%, or about 55.0% of the PEG 300.

In some embodiments, the composition comprises about 5% to about 10% of the poloxamer 188, for example, about 5.0%, about 5.1%, about 5.2%, about 5.3%, about 5.4%, about 5.5%, about 5.6%, about 5.7%, about 5.8%, about 5.9%, about 6.0%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0% of the poloxamer 188.

In some embodiments, the composition comprises about 5% to about 20% of the water, for example, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0%, about 10.1%, about 10.2%, about 10.3%, about 10.4%, about 10.5%, about 10.6%, about 10.7%, about 10.8%, about 10.9%, about 11.0%, about 11.1%, about 11.2%, about 11.3%, about 11.4%, about 11.5%, about 11.6%, about 11.7%, about 11.8%, about 11.9%, about 12.0%, about 12.1%, about 12.2%, about 12.3%, about 12.4%, about 12.5%, about 12.6%, about 12.7%, about 12.8%, about 12.9%, about 13.0%, about 13.1%, about 13.2%, about 13.3%, about 13.4%, about 13.5%, about 13.6%, about 13.7%, about 13.8%, about 13.9%, about 14.0%, about 14.1%, about 14.2%, about 14.3%, about 14.4%, about 14.5%, about 14.6%, about 14.7%, about 14.8%, about 14.9%, about 15.0%, about 15.1%, about 15.2%, about 15.3%, about 15.4%, about 15.5%, about 15.6%, about 15.7%, about 15.8%, about 15.9%, about 16.0%, about 16.1%, about 16.2%, about 16.3%, about 16.4%, about 16.5%, about 16.6%, about 16.7%, about 16.8%, about 16.9%, about 17.0%, about 17.1%, about 17.2%, about 17.3%, about 17.4%, about 17.5%, about 17.6%, about 17.7%, about 17.8%, about 17.9%, about 18.0%, about 18.1%, about 18.2%, about 18.3%, about 18.4%, about 18.5%, about 18.6%, about 18.7%, about 18.8%, about 18.9%, about 19.0%, about 19.1%, about 19.2%, about 19.3%, about 19.4%, about 19.5%, about 19.6%, about 19.7%, about 19.8%, about 19.9%, or about 20.0% of the water.

In some embodiments, the composition comprises about 3% to about 15% of the ethanol, for example, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0%, about 10.1%, about 10.2%, about 10.3%, about 10.4%, about 10.5%, about 10.6%, about 10.7%, about 10.8%, about 10.9%, about 11.0%, about 11.1%, about 11.2%, about 11.3%, about 11.4%, about 11.5%, about 11.6%, about 11.7%, about 11.8%, about 11.9%, about 12.0%, about 12.1%, about 12.2%, about 12.3%, about 12.4%, about 12.5%, about 12.6%, about 12.7%, about 12.8%, about 12.9%, about 13.0%, about 13.1%, about 13.2%, about 13.3%, about 13.4%, about 13.5%, about 13.6%, about 13.7%, about 13.8%, about 13.9%, about 14.0%, about 14.1%, about 14.2%, about 14.3%, about 14.4%, about 14.5%, about 14.6%, about 14.7%, about 14.8%, about 14.9%, or about 15.0% of the ethanol.

In some embodiments, the present disclosure relates to a pharmaceutical composition comprising a free acid form of the compound of Formula Ia:

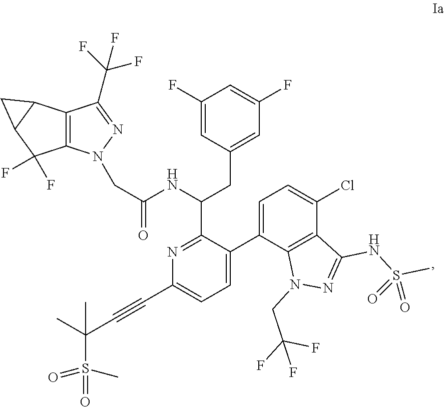 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 30% to about 45% of the free acid form of the compound of Formula Ia;
about 37% to about 50% of the PEG 300;
about 5% to about 10% of the water; and
about 7% to about 15% of the ethanol.

In some embodiments, the present disclosure relates to a pharmaceutical composition comprising a free acid form of the compound of Formula Ia:

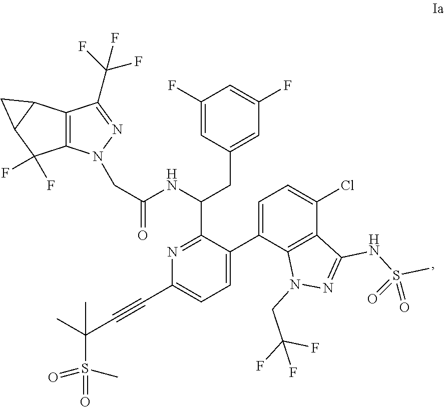 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 30% to about 45% of the free acid form of the compound of Formula Ia;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ia:

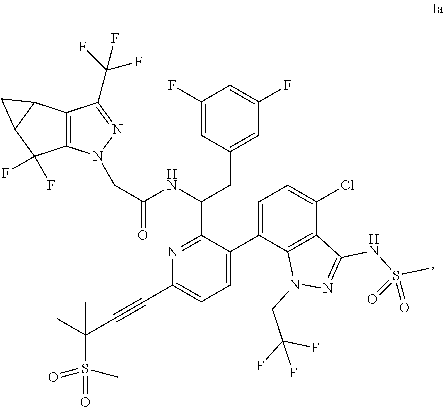 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 41% to about 43% of the free acid form of the compound of Formula Ia;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ia:

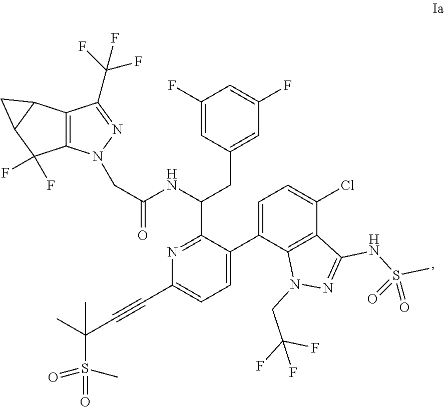 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 42% of the free acid form of the compound of Formula Ia;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ia:

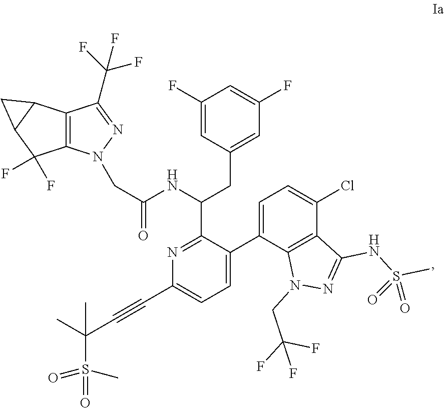 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 42.02% of the free acid form of the compound of Formula Ia;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ia:

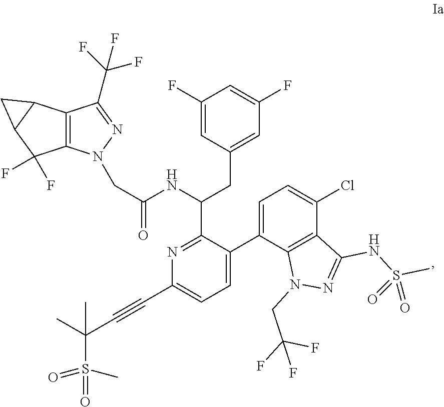 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 30% to about 45% of the free acid form of the compound of Formula Ia;
about 40% to about 50% of the PEG 300;
about 5% to about 10% of the water; and
about 7% to about 15% of the ethanol.

In some embodiments, the present disclosure relates to a pharmaceutical composition comprising a free acid form of the compound of Formula Ib:

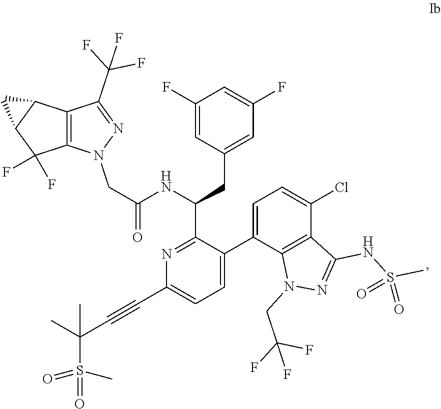 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 30% to about 45% of the free acid form of the compound of Formula Ib;
about 37% to about 50% of the PEG 300;
about 5% to about 10% of the water; and
about 7% to about 15% of the ethanol.

In some embodiments, the present disclosure relates to a pharmaceutical composition comprising a free acid form of the compound of Formula Ib:

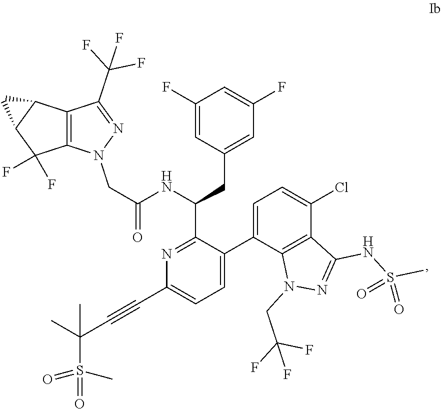 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 30% to about 45% of the free acid form of the compound of Formula Ib;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ib:

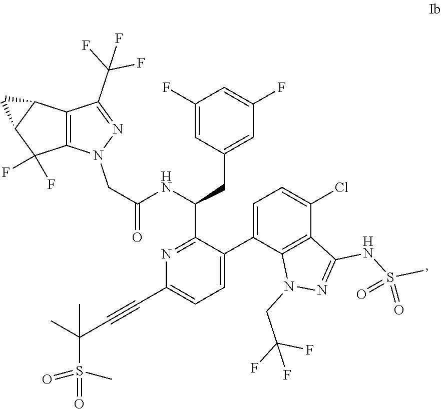 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 41% to about 43% of the free acid form of the compound of Formula Ib;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ib:

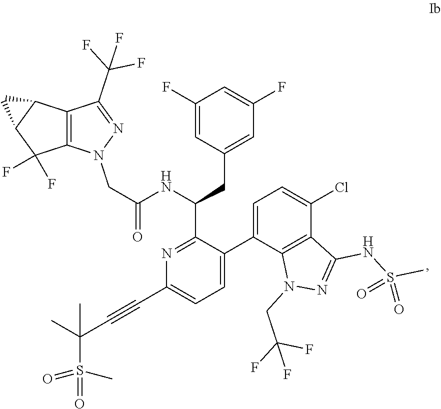 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 42% of the free acid form of the compound of Formula Ib;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ib:

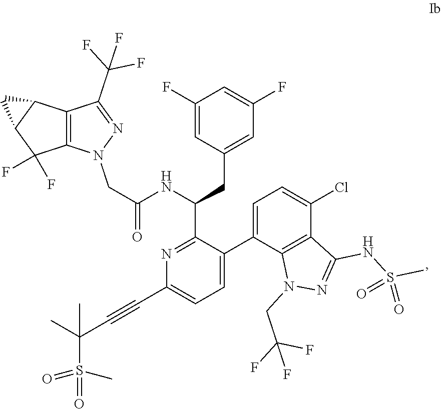 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 42.02% of the free acid form of the compound of Formula Ib;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ib:

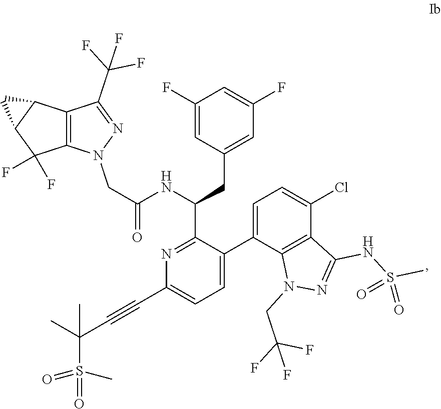 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 30% to about 45% of the free acid form of the compound of Formula Ib;
about 40% to about 50% of the PEG 300;
about 5% to about 10% of the water; and
about 7% to about 15% of the ethanol.

In some embodiments, the present disclosure relates to a pharmaceutical composition comprising:
about 30% to about 45% of a means for inhibiting HIV capsid;
about 40% to about 50% of the PEG 300;
about 5% to about 10% of the water; and
about 7% to about 15% of the ethanol.

In some embodiments, the present disclosure relates to a pharmaceutical composition comprising:
about 30% to about 45% of a means for inhibiting HIV capsid;
about 37% to about 50% of the PEG 300;
about 5% to about 10% of the water; and
about 7% to about 15% of the ethanol.

In some embodiments, the present disclosure relates to a pharmaceutical composition comprising:
about 30% to about 45% of a means for inhibiting HIV capsid;
about 37% to about 43% of the PEG 300;
about 6% to about 8% of the water; and
about 9% to about 11% of the ethanol.

In some embodiments, the present disclosure relates to a pharmaceutical composition comprising:
about 41% to about 43% of a means for inhibiting HIV capsid;
about 37% to about 43% of the PEG 300;
about 6% to about 8% of the water; and
about 9% to about 11% of the ethanol.

In some embodiments, the present disclosure relates to a pharmaceutical composition comprising:
about 42% of a means for inhibiting HIV capsid;
about 37% to about 43% of the PEG 300;
about 6% to about 8% of the water; and
about 9% to about 11% of the ethanol.

In some embodiments, the present disclosure relates to a pharmaceutical composition comprising:
about 42.02% of a means for inhibiting HIV capsid;
about 37% to about 43% of the PEG 300;
about 6% to about 8% of the water; and
about 9% to about 11% of the ethanol.

### Composition 1

In some embodiments, the present disclosure relates to a pharmaceutical composition comprising a compound of Formula Ia:

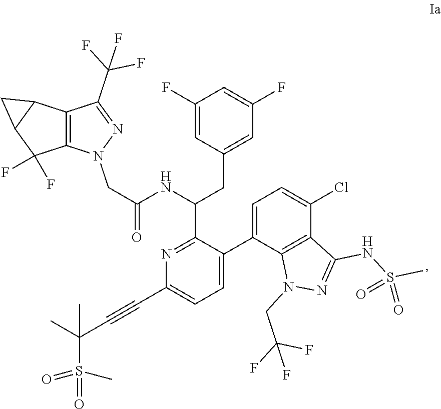 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, PEG 300, ethanol, and water, wherein the composition comprises:
about 30% to about 45% of the compound of Formula Ia, or a pharmaceutically acceptable salt thereof;
about 35% to about 45% of the PEG 300;
about 15% to about 25% of the water; and
about 2% to about 7% of the ethanol.

In some embodiments, the present disclosure relates to a pharmaceutical composition comprising a compound of Formula Ib:

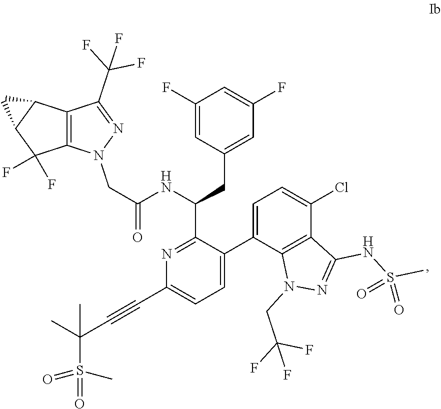 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, PEG 300, ethanol, and water, wherein the composition comprises:
about 30% to about 45% of the compound of Formula Ia, or a pharmaceutically acceptable salt thereof;
about 35% to about 45% of the PEG 300;
about 15% to about 25% of the water; and
about 2% to about 7% of the ethanol.

In some embodiments, the present disclosure relates to a pharmaceutical composition comprising a compound of Formula Ia:

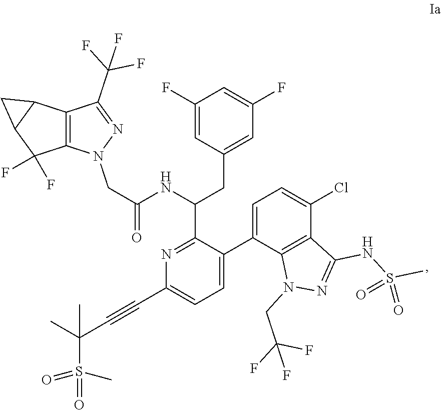 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, PEG 300, water, and ethanol, wherein the composition comprises:
about 30% to about 45% of the compound of Formula Ia, or a pharmaceutically acceptable salt thereof;
about 40% to about 50% of the PEG 300;
about 5% to about 10% of the water; and
about 7% to about 15% of the ethanol.

In some embodiments, the present disclosure relates to a pharmaceutical composition comprising a compound of Formula Ib:

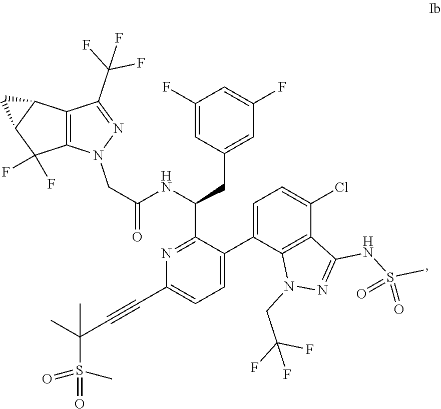 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, PEG 300, water, and ethanol, wherein the composition comprises:
about 30% to about 45% of the compound of Formula Ib, or a pharmaceutically acceptable salt thereof;
about 40% to about 50% of the PEG 300;
about 5% to about 10% of the water; and
about 7% to about 15% of the ethanol.

In some embodiments, the composition comprises about 33% to about 43% of the compound of Formula Ia or Formula Ib, or a pharmaceutically acceptable salt thereof, for example, about 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, about 35.0%, about 35.1%, about 35.2%, about 35.3%, about 35.4%, about 35.5%, about 35.6%, about 35.7%, about 35.8%, about 35.9%, about 36.0%, about 36.1%, about 36.2%, about 36.3%, about 36.4%, about 36.5%, about 36.6%, about 36.7%, about 36.8%, about 36.9%, about 37.0%, about 37.1%, about 37.2%, about 37.3%, about 37.4%, about 37.5%, about 37.6%, about 37.7%, about 37.8%, about 37.9%, about 38.0%, about 38.1%, about 38.2%, about 38.3%, about 38.4%, about 38.5%, about 38.6%, about 38.7%, about 38.8%, about 38.9%, about 39.0%, about 39.1%, about 39.2%, about 39.3%, about 39.4%, about 39.5%, about 39.6%, about 39.7%, about 39.8%, about 39.9%, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, or about 43.0%, of the compound of Formula Ia or Formula Ib, or a pharmaceutically acceptable salt thereof.

In some embodiments, the composition comprises about 33% to about 36% of the compound of Formula Ia or Formula Ib, or a pharmaceutically acceptable salt thereof, for example, about 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, about 35.0%, about 35.1%, about 35.2%, about 35.3%, about 35.4%, about 35.5%, about 35.6%, about 35.7%, about 35.8%, about 35.9%, or about 36.0% of the compound of Formula Ia or Formula Ib, or a pharmaceutically acceptable salt thereof.

In some embodiments, the composition comprises about 34% to about 35% of the compound of Formula Ia or Formula Ib, or a pharmaceutically acceptable salt thereof. In some embodiments, the composition comprises about 34.25% to about 34.75% of the compound of Formula Ia or Formula Ib, or a pharmaceutically acceptable salt thereof. In some embodiments, the composition comprises about 34.5% of the compound of Formula Ia or Formula Ib, or a pharmaceutically acceptable salt thereof. In some embodiments, the composition comprises about 34.50% of the compound of Formula Ia or Formula Ib, or a pharmaceutically acceptable salt thereof.

In some embodiments, the composition comprises about 35% to about 45% of the PEG 300. In some embodiments, the composition comprises about 40% to about 43% of the PEG 300, for example, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, or about 43.0% of the PEG 300.

In some embodiments, the composition comprises about 41% to about 42% of the PEG 300. In some embodiments, the composition comprises about 41.1% to about 41.3% of the PEG 300. In some embodiments, the composition comprises about 41.2% of the PEG 300. In some embodiments, the composition comprises about 41.24% of the PEG 300.

In some embodiments, the composition comprises about 15% to about 25% of the water. In some embodiments, the composition comprises about 15% to about 20% of the water, for example, about 15.0%, about 15.1%, about 15.2%, about 15.3%, about 15.4%, about 15.5%, about 15.6%, about 15.7%, about 15.8%, about 15.9%, about 16.0%, about 16.1%, about 16.2%, about 16.3%, about 16.4%, about 16.5%, about 16.6%, about 16.7%, about 16.8%, about 16.9%, about 17.0%, about 17.1%, about 17.2%, about 17.3%, about 17.4%, about 17.5%, about 17.6%, about 17.7%, about 17.8%, about 17.9%, about 18.0%, about 18.1%, about 18.2%, about 18.3%, about 18.4%, about 18.5%, about 18.6%, about 18.7%, about 18.8%, about 18.9%, about 19.0%, about 19.1%, about 19.2%, about 19.3%, about 19.4%, about 19.5%, about 19.6%, about 19.7%, about 19.8%, about 19.9%, or about 20.0% of the water.

In some embodiments, the composition comprises about 19% to about 20% of the water. In some embodiments, the composition comprises about 19.1% to about 19.3% of the water. In some embodiments, the composition comprises about 19.3% of the water. In some embodiments, the composition comprises about 19.26% of the water.

In some embodiments, the composition comprises about 2% to about 7% of the ethanol. In some embodiments, the composition comprises about 4% to about 6% of the ethanol, for example, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, or about 6.0% of the ethanol.

In some embodiments, the composition comprises about 4.5% to about 5.5% of the ethanol. In some embodiments, the composition comprises about 4.75% to about 5.25% of the ethanol. In some embodiments, the composition comprises about 5% of the ethanol. In some embodiments, the composition comprises about 5.0% of the ethanol. In some embodiments, the composition comprises about 5.00% of the ethanol.

In some embodiments, the composition comprises:
about 33% to about 36% of the compound of Formula Ia or Formula Ib, or a pharmaceutically acceptable salt thereof;
about 40% to about 43% of the PEG 300;
about 15% to about 20% of the water; and
about 4% to about 6% of the ethanol.

In some embodiments, the composition comprises:
about 34% to about 35% of the compound of Formula Ia or Formula Ib, or a pharmaceutically acceptable salt thereof;
about 40% to about 43% of the PEG 300;
about 15% to about 20% of the water; and
about 4% to about 6% of the ethanol.

In some embodiments, the composition comprises:
about 34.25% to about 34.75% of the compound of Formula Ia or Formula Ib, or a pharmaceutically acceptable salt thereof;
about 41.1% to about 41.3% of the PEG 300;
about 19.1% to about 19.3% of the water; and
about 4.75% to about 5.25% of the ethanol.

In some embodiments, the composition comprises:
about 34.5% of the compound of Formula Ia or Formula Ib, or a pharmaceutically acceptable salt thereof;
about 41.2% of the PEG 300;
about 19.3% of the water; and
about 5.0% of the ethanol.

In some embodiments, the composition comprises:
about 34.50% of the compound of Formula Ia or Formula Ib, or a pharmaceutically acceptable salt thereof;
about 41.24% of the PEG 300;
about 19.26% of the water; and
about 5.00% of the ethanol.

In some embodiments, the composition comprises:
about 33% to about 36% of the sodium salt of the compound of Formula Ia or Formula Ib;
about 40% to about 43% of the PEG 300;
about 15% to about 20% of the water; and
about 4% to about 6% of the ethanol.

In some embodiments, the composition comprises:
about 34% to about 35% of the sodium salt of the compound of Formula Ia or Formula Ib;
about 40% to about 43% of the PEG 300;
about 15% to about 20% of the water; and
about 4% to about 6% of the ethanol.

In some embodiments, the composition comprises:
about 34.25% to about 34.75% of the sodium salt of the compound of Formula Ia or Formula Ib;
about 41.1% to about 41.3% of the PEG 300;
about 19.1% to about 19.3% of the water; and
about 4.75% to about 5.25% of the ethanol.

In some embodiments, the composition comprises:
about 34.5% of the sodium salt of the compound of Formula Ia or Formula Ib;
about 41.2% of the PEG 300;
about 19.3% of the water; and
about 5.0% of the ethanol.

In some embodiments, the composition comprises:
about 34.50% of the sodium salt of the compound of Formula Ia or Formula Ib;
about 41.24% of the PEG 300;
about 19.26% of the water; and
about 5.00% of the ethanol.

In some embodiments, the composition consists of:
about 33% to about 36% of the compound of Formula Ia or Formula Ib, or a pharmaceutically acceptable salt thereof;
about 40% to about 43% of the PEG 300;
about 15% to about 20% of the water; and
about 4% to about 6% of the ethanol.

In some embodiments, the composition consists of:
about 34% to about 35% of the compound of Formula Ia or Formula Ib, or a pharmaceutically acceptable salt thereof;
about 40% to about 43% of the PEG 300;
about 15% to about 20% of the water; and
about 4% to about 6% of the ethanol.

In some embodiments, the composition consists of:
about 34.25% to about 34.75% of the compound of Formula Ia or Formula Ib, or a pharmaceutically acceptable salt thereof;
about 41.1% to about 41.3% of the PEG 300;
about 19.1% to about 19.3% of the water; and
about 4.75% to about 5.25% of the ethanol.

In some embodiments, the composition consists of:
about 34.5% of the compound of Formula Ia or Formula Ib, or a pharmaceutically acceptable salt thereof;
about 41.2% of the PEG 300;
about 19.3% of the water; and
about 5.0% of the ethanol.

In some embodiments, the composition consists of:
about 34.50% of the compound of Formula Ia or Formula Ib, or a pharmaceutically acceptable salt thereof;
about 41.24% of the PEG 300;
about 19.26% of the water; and
about 5.00% of the ethanol.

In some embodiments, the composition consists of:
about 33% to about 36% of the sodium salt of the compound of Formula Ia or Formula Ib;
about 40% to about 43% of the PEG 300;
about 15% to about 20% of the water; and
about 4% to about 6% of the ethanol.

In some embodiments, the composition consists of:
about 34% to about 35% of the sodium salt of the compound of Formula Ia or Formula Ib;
about 40% to about 43% of the PEG 300;
about 15% to about 20% of the water; and about 4% to about 6% of the ethanol.

In some embodiments, the composition consists of:
about 34.25% to about 34.75% of the sodium salt of the compound of Formula Ia or Formula Ib;
about 41.1% to about 41.3% of the PEG 300;
about 19.1% to about 19.3% of the water; and
about 4.75% to about 5.25% of the ethanol.

In some embodiments, the composition consists of:
about 34.5% of the sodium salt of the compound of Formula Ia or Formula Ib;
about 41.2% of the PEG 300;
about 19.3% of the water; and
about 5.0% of the ethanol.

In some embodiments, the composition consists of:
about 34.50% of the sodium salt of the compound of Formula Ia or Formula Ib;
about 41.24% of the PEG 300;
about 19.26% of the water; and about 5.00% of the ethanol.

### Composition 2

In some embodiments, the present disclosure relates to a pharmaceutical composition comprising a compound of Formula Ia:

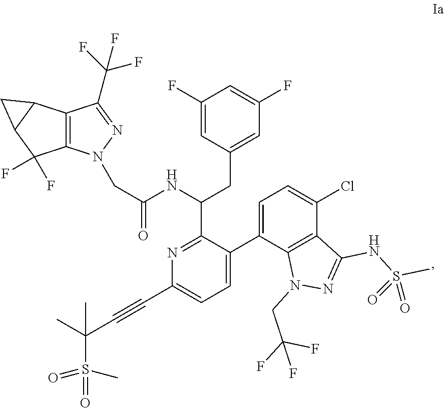 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, PEG 300, ethanol, and water, wherein the composition comprises:
about 30% to about 45% of the compound of Formula Ia, or a pharmaceutically acceptable salt thereof;
about 30% to about 40% of the PEG 300;
about 10% to about 20% of the water; and
about 5% to about 10% of the ethanol.

In some embodiments, the present disclosure relates to a pharmaceutical composition comprising a compound of Formula Ib:

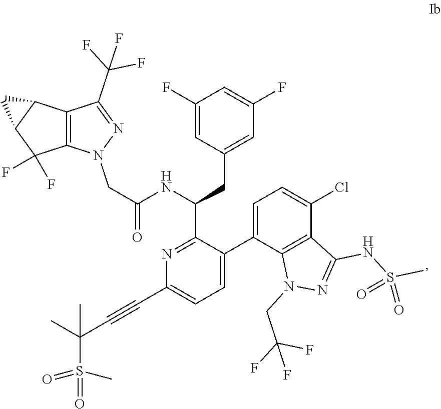 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, PEG 300, ethanol, and water, wherein the composition comprises:
about 30% to about 45% of the compound of Formula Ib, or a pharmaceutically acceptable salt thereof;
about 30% to about 40% of the PEG 300;
about 10% to about 20% of the water; and
about 5% to about 10% of the ethanol.

In some embodiments, the composition comprises about 40% to about 43% of the compound of Formula Ia or Formula Ib, or a pharmaceutically acceptable salt thereof, for example, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, or about 43.0% of the compound of Formula Ia or Formula Ib, or a pharmaceutically acceptable salt thereof.

In some embodiments, the composition comprises about 40% to about 43% of the sodium salt of the compound of Formula Ia or Formula Ib, for example, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, or about 43.0% of the sodium salt of the compound of Formula Ia or Formula Ib.

In some embodiments, the composition comprises about 41% to about 42% of the compound of Formula Ia or Formula Ib, or a pharmaceutically acceptable salt thereof. In some embodiments, the composition comprises about 41.5% to about 42.0% of the compound of Formula Ia or Formula Ib, or a pharmaceutically acceptable salt thereof. In some embodiments, the composition comprises about 41.8% to about 42.9% of the compound of Formula Ia or Formula Ib, or a pharmaceutically acceptable salt thereof. In some embodiments, the composition comprises about 41.8% of the compound of Formula Ia or Formula Ib, or a pharmaceutically acceptable salt thereof. In some embodiments, the composition comprises about 41.9% of the compound of Formula Ia or Formula Ib, or a pharmaceutically acceptable salt thereof. In some embodiments, the composition comprises about 41.85% of the compound of Formula Ia or Formula Ib, or a pharmaceutically acceptable salt thereof.

In some embodiments, the composition comprises about 41% to about 42% of the sodium salt of the compound of Formula Ia or Formula Ib. In some embodiments, the composition comprises about 41.5% to about 42.0% of the sodium salt of the compound of Formula Ia or Formula Ib. In some embodiments, the composition comprises about 41.8% to about 42.9% of the sodium salt of the compound of Formula Ia or Formula Ib. In some embodiments, the composition comprises about 41.8% of the sodium salt of the compound of Formula Ia or Formula Ib. In some embodiments, the composition comprises about 41.9% of the sodium salt of the compound of Formula Ia or Formula Ib. In some embodiments, the composition comprises about 41.85% of the sodium salt of the compound of Formula Ia or Formula Ib.

In some embodiments, the composition comprises about 35% to about 37% of the PEG 300, for example, about 35.0%, about 35.1%, about 35.2%, about 35.3%, about 35.4%, about 35.5%, about 35.6%, about 35.7%, about 35.8%, about 35.9%, about 36.0%, about 36.1%, about 36.2%, about 36.3%, about 36.4%, about 36.5%, about 36.6%, about 36.7%, about 36.8%, about 36.9%, or about 37.0% of the PEG 300.

In some embodiments, the composition comprises about 36% to about 37% of the PEG 300. In some embodiments, the composition comprises about 36.0% to about 36.5% of the PEG 300. In some embodiments, the composition comprises about 36.1% to about 36.3% of the PEG 300. In some embodiments, the composition comprises about 36.2% of the PEG 300. In some embodiments, the composition comprises about 36.22% of the PEG 300.

In some embodiments, the composition comprises about 15% to about 20% of the water, for example, about 15.0%, about 15.1%, about 15.2%, about 15.3%, about 15.4%, about 15.5%, about 15.6%, about 15.7%, about 15.8%, about 15.9%, about 16.0%, about 16.1%, about 16.2%, about 16.3%, about 16.4%, about 16.5%, about 16.6%, about 16.7%, about 16.8%, about 16.9%, about 17.0%, about 17.1%, about 17.2%, about 17.3%, about 17.4%, about 17.5%, about 17.6%, about 17.7%, about 17.8%, about 17.9%, about 18.0%, about 18.1%, about 18.2%, about 18.3%, about 18.4%, about 18.5%, about 18.6%, about 18.7%, about 18.8%, about 18.9%, about 19.0%, about 19.1%, about 19.2%, about 19.3%, about 19.4%, about 19.5%, about 19.6%, about 19.7%, about 19.8%, about 19.9%, or about 20.0% of the water.

In some embodiments, the composition comprises about 15% to about 17% of the water. In some embodiments, the composition comprises about 16% to about 17% of the water. In some embodiments, the composition comprises about 16.5% to about 17% of the water. In some embodiments, the composition comprises about 16.9% of the water. In some embodiments, the composition comprises about 16.93% of the water.

In some embodiments, the composition comprises about 4% to about 6% of the ethanol, for example, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, or about 6.0% of the ethanol.

In some embodiments, the composition comprises about 4.5% to about 5.5% of the ethanol. In some embodiments, the composition comprises about 4.9% to about 5.1% of the ethanol. In some embodiments, the composition comprises about 5.0% of the ethanol. In some embodiments, the composition comprises about 5.00% of the ethanol.

In some embodiments, the composition comprises:
about 40% to about 43% of the compound of Formula Ia or Formula Ib, or a pharmaceutically acceptable salt thereof;
about 35% to about 37% of the PEG 300;
about 15% to about 20% of the water; and
about 4% to about 6% of the ethanol.

In some embodiments, the composition comprises:
about 41% to about 42% of the compound of Formula Ia or Formula Ib, or a pharmaceutically acceptable salt thereof;
about 35% to about 37% of the PEG 300;
about 15% to about 17% of the water; and
about 4% to about 6% of the ethanol.

In some embodiments, the composition comprises:
about 41.5% to about 42.0% of the compound of Formula Ia or Formula Ib, or a pharmaceutically acceptable salt thereof;
about 36.0% to about 36.5% of the PEG 300;
about 16.5% to about 17% of the water; and
about 4% to about 6% of the ethanol.

In some embodiments, the composition comprises:
about 41.8% of the compound of Formula Ia or Formula Ib, or a pharmaceutically acceptable salt thereof;
about 36.2% of the PEG 300;
about 16.9% of the water; and
about 5.0% of the ethanol.

In some embodiments, the composition comprises:
about 41.9% of the compound of Formula Ia or Formula Ib, or a pharmaceutically acceptable salt thereof;
about 36.2% of the PEG 300;
about 16.9% of the water; and
about 5.0% of the ethanol.

In some embodiments, the composition comprises:
about 41.85% of the compound of Formula Ia or Formula Ib, or a pharmaceutically acceptable salt thereof;
about 36.22% of the PEG 300;
about 16.93% of the water; and
about 5.00% of the ethanol.

In some embodiments, the composition comprises:
about 40% to about 43% of the sodium salt of the compound of Formula Ia or Formula Ib;
about 35% to about 37% of the PEG 300;
about 15% to about 20% of the water; and
about 4% to about 6% of the ethanol.

In some embodiments, the composition comprises:
about 41% to about 42% of the sodium salt of the compound of Formula Ia or Formula Ib;
about 35% to about 37% of the PEG 300;
about 15% to about 17% of the water; and
about 4% to about 6% of the ethanol.

In some embodiments, the composition comprises:
about 41.5% to about 42.0% of the sodium salt of the compound of Formula Ia or Formula Ib;
about 36.0% to about 36.5% of the PEG 300;
about 16.5% to about 17% of the water; and
about 4% to about 6% of the ethanol.

In some embodiments, the composition comprises:
about 41.8% of the sodium salt of the compound of Formula Ia or Formula Ib; about 36.2% of the PEG 300;
about 16.9% of the water; and
about 5.0% of the ethanol.

In some embodiments, the composition comprises:
about 41.9% of the sodium salt of the compound of Formula Ia or Formula Ib;
about 36.2% of the PEG 300;
about 16.9% of the water; and
about 5.0% of the ethanol.

In some embodiments, the composition comprises:
about 41.85% of the sodium salt of the compound of Formula Ia or Formula Ib;
about 36.22% of the PEG 300;
about 16.93% of the water; and
about 5.00% of the ethanol.

In some embodiments, the composition consists of:
about 40% to about 43% of the compound of Formula Ia or Formula Ib, or a pharmaceutically acceptable salt thereof;
about 35% to about 37% of the PEG 300;
about 15% to about 20% of the water; and
about 4% to about 6% of the ethanol.

In some embodiments, the composition consists of:
about 41% to about 42% of the compound of Formula Ia or Formula Ib, or a pharmaceutically acceptable salt thereof;
about 35% to about 37% of the PEG 300;
about 15% to about 17% of the water; and
about 4% to about 6% of the ethanol.

In some embodiments, the composition consists of:
about 41.5% to about 42.0% of the compound of Formula Ia or Formula Ib, or a pharmaceutically acceptable salt thereof;
about 36.0% to about 36.5% of the PEG 300;
about 16.5% to about 17% of the water; and
about 4% to about 6% of the ethanol.

In some embodiments, the composition consists of:
about 41.8% of the compound of Formula Ia or Formula Ib, or a pharmaceutically acceptable salt thereof;
about 36.2% of the PEG 300;
about 16.9% of the water; and
about 5.0% of the ethanol.

In some embodiments, the composition consists of:
about 41.9% of the compound of Formula Ia or Formula Ib, or a pharmaceutically acceptable salt thereof;
about 36.2% of the PEG 300;
about 16.9% of the water; and
about 5.0% of the ethanol.

In some embodiments, the composition consists of:
about 41.85% of the compound of Formula Ia or Formula Ib, or a pharmaceutically acceptable salt thereof;
about 36.22% of the PEG 300;
about 16.93% of the water; and
about 5.00% of the ethanol.

In some embodiments, the composition consists of:
about 40% to about 43% of the sodium salt of the compound of Formula Ia or Formula Ib;
about 35% to about 37% of the PEG 300;
about 15% to about 20% of the water; and
about 4% to about 6% of the ethanol.

In some embodiments, the composition consists of:
about 41% to about 42% of the sodium salt of the compound of Formula Ia or Formula Ib;
about 35% to about 37% of the PEG 300;
about 15% to about 17% of the water; and
about 4% to about 6% of the ethanol.

In some embodiments, the composition consists of:
about 41.5% to about 42.0% of the sodium salt of the compound of Formula Ia or Formula Ib;
about 36.0% to about 36.5% of the PEG 300;
about 16.5% to about 17% of the water; and
about 4% to about 6% of the ethanol.

In some embodiments, the composition consists of:
about 41.8% of the sodium salt of the compound of Formula Ia or Formula Ib; about 36.2% of the PEG 300;
about 16.9% of the water; and
about 5.0% of the ethanol.

In some embodiments, the composition consists of:
about 41.9% of the sodium salt of the compound of Formula Ia or Formula Ib;
about 36.2% of the PEG 300;
about 16.9% of the water; and
about 5.0% of the ethanol.

In some embodiments, the composition consists of:
about 41.85% of the sodium salt of the compound of Formula Ia or Formula Ib;
about 36.22% of the PEG 300;
about 16.93% of the water; and
about 5.00% of the ethanol.

### Composition 3

In some embodiments, the present disclosure relates to a pharmaceutical composition comprising a compound of Formula Ia:

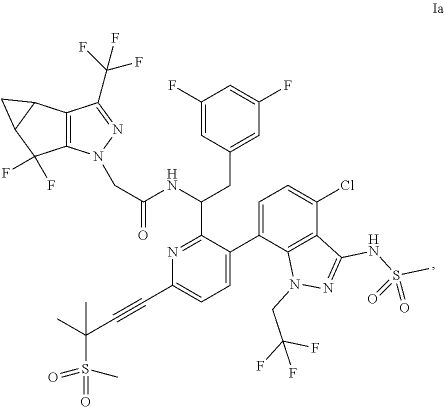 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, ethanol, and water, wherein the composition comprises:
about 30% to about 45% of the free acid form of the compound of Formula Ia;
about 37% to about 50% of the PEG 300;
about 5% to about 10% of the water; and
about 7% to about 15% of the ethanol.

In some embodiments, the present disclosure relates to a pharmaceutical composition comprising a compound of Formula Ia:

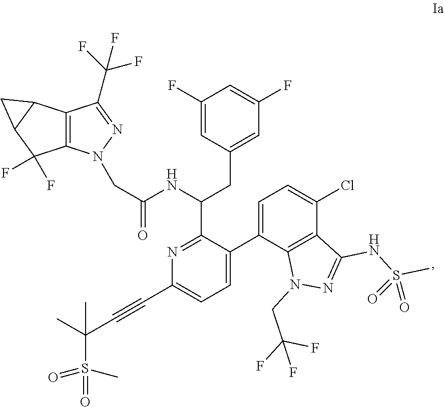 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, ethanol, and water, wherein the composition comprises:
about 30% to about 45% of the free acid form of the compound of Formula Ia;
about 40% to about 50% of the PEG 300;
about 5% to about 10% of the water; and
about 7% to about 15% of the ethanol.

In some embodiments, the present disclosure relates to a pharmaceutical composition comprising a free acid form of the compound of Formula Ia:

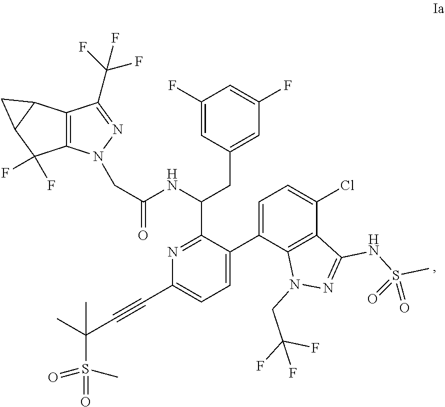 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 41% to about 43% of the free acid form of the compound of Formula Ia;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ia:

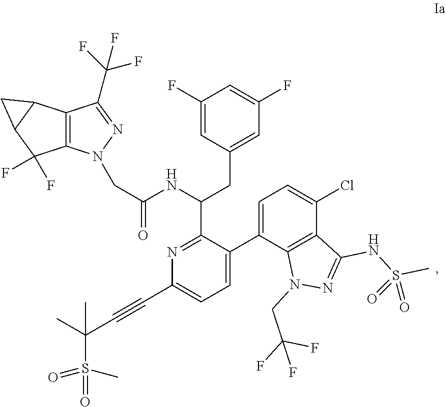 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 42% of the free acid form of the compound of Formula Ia;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ia:

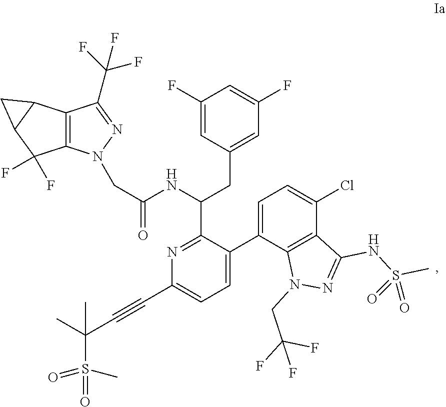 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 42.02% of the free acid form of the compound of Formula Ia;
about 37% to about 43% of the PEG 300;
about 6% to about 8% of the water; and
about 9% to about 11% of the ethanol.

In some embodiments, the present disclosure relates to a pharmaceutical composition comprising a compound of Formula Ib:

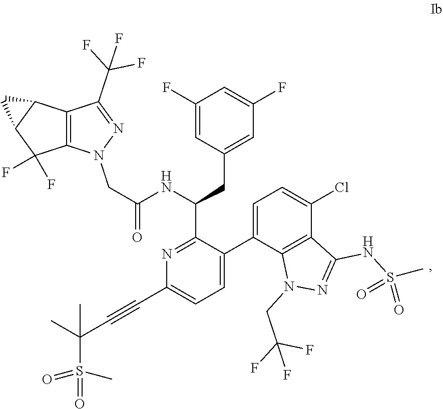 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, ethanol, and water, wherein the composition comprises:
about 30% to about 45% of the free acid form of the compound of Formula Ib;
about 37% to about 50% of the PEG 300;
about 5% to about 10% of the water; and
about 7% to about 15% of the ethanol.

In some embodiments, the present disclosure relates to a pharmaceutical composition comprising a compound of Formula Ib:

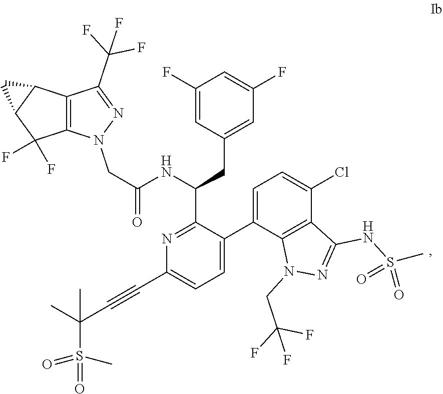 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, ethanol, and water, wherein the composition comprises:
about 30% to about 45% of the free acid form of the compound of Formula Ib;
about 40% to about 50% of the PEG 300;
about 5% to about 10% of the water; and
about 7% to about 15% of the ethanol.

In some embodiments, the present disclosure relates to a pharmaceutical composition comprising a free acid form of the compound of Formula Ib:

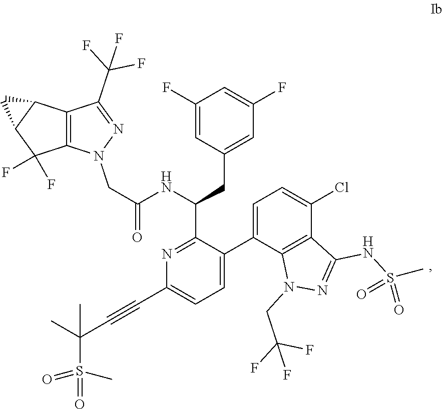 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 41% to about 43% of the free acid form of the compound of Formula Ib;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ib:

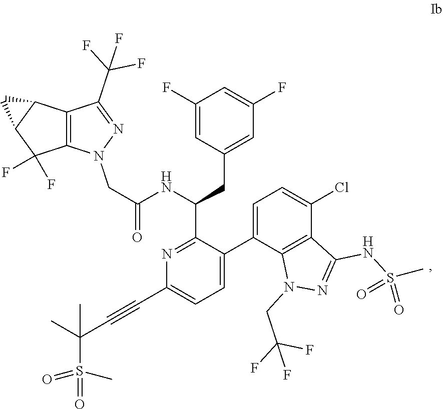 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 42% of the free acid form of the compound of Formula Ib;
about 37% to about 43% of the PEG 300;
about 6% to about 8% of the water; and
about 9% to about 11% of the ethanol.

In some embodiments, the present disclosure relates to a pharmaceutical composition comprising a free acid form of the compound of Formula Ib:

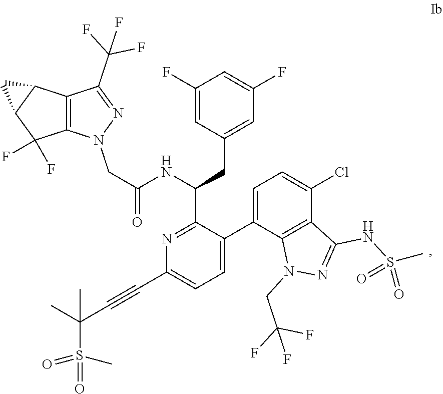 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 42.02% of the free acid form of the compound of Formula Ib;
about 37% to about 43% of the PEG 300;
about 6% to about 8% of the water; and
about 9% to about 11% of the ethanol.

In some embodiments, the composition comprises about 40% to about 43% of the free acid form of the compound of Formula Ia or Formula Ib, for example, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, about 43.0%, about 43.1%, about 43.2%, about 43.3%, about 43.4%, about 43.5%, about 43.6%, about 43.7%, about 43.8%, about 43.9%, about 44.0%, about 44.1%, about 44.2%, about 44.3%, about 44.4%, about 44.5%, about 44.6%, about 44.7%, about 44.8%, about 44.9%, or about 45.0% of the free acid form of the compound of Formula Ia or Formula Ib.

In some embodiments, the composition comprises about 41% to about 43% of the free acid form of the compound of Formula Ia or Formula Ib. In some embodiments, the composition comprises about 42% to about 43% of the free acid form of the compound of Formula Ia or Formula Ib.

In some embodiments, the composition comprises about 41.5% to about 42.5% of the free acid form of the compound of Formula Ia or Formula Ib. In some embodiments, the composition comprises about 41.75% to about 42.25% of the free acid form of the compound of Formula Ia or Formula Ib. In some embodiments, the composition comprises about 42.0% to about 42.1% of the free acid form of the compound of Formula Ia or Formula Ib.

In some embodiments, the composition comprises about 42% of the free acid form of the compound of Formula Ia or Formula Ib. In some embodiments, the composition comprises about 42.0% of the free acid form of the compound of Formula Ia or Formula Ib. In some embodiments, the composition comprises about 42.02% of the free acid form of the compound of Formula Ia or Formula Ib.

In some embodiments, the composition comprises about 37% to about 43% of the PEG 300, for example, about 37.0%, about 37.1%, about 37.2%, about 37.3%, about 37.4%, about 37.5%, about 37.6%, about 37.7%, about 37.8%, about 37.9%, about 38.0%, about 38.1%, about 38.2%, about 38.3%, about 38.4%, about 38.5%, about 38.6%, about 38.7%, about 38.8%, about 38.9%, about 39.0%, about 39.1%, about 39.2%, about 39.3%, about 39.4%, about 39.5%, about 39.6%, about 39.7%, about 39.8%, about 39.9%, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, about 43.0%, about 43.1%, about 43.2%, about 43.3%, about 43.4%, about 43.5%, about 43.6%, about 43.7%, about 43.8%, about 43.9%, about 44.0%, about 44.1%, about 44.2%, about 44.3%, about 44.4%, about 44.5%, about 44.6%, about 44.7%, about 44.8%, about 44.9%, or about 45.0% of the PEG 300.

In some embodiments, the composition comprises about 40% to about 45% of the PEG 300, for example, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, about 43.0%, about 43.1%, about 43.2%, about 43.3%, about 43.4%, about 43.5%, about 43.6%, about 43.7%, about 43.8%, about 43.9%, about 44.0%, about 44.1%, about 44.2%, about 44.3%, about 44.4%, about 44.5%, about 44.6%, about 44.7%, about 44.8%, about 44.9%, or about 45.0% of the PEG 300.

In some embodiments, the composition comprises about 40% to about 41% of the PEG 300. In some embodiments, the composition comprises about 40.5% to about 41% of the PEG 300. In some embodiments, the composition comprises about 41% of the PEG 300. In some embodiments, the composition comprises about 40.7% to about 40.8% of the PEG 300. In some embodiments, the composition comprises about 40.7% of the PEG 300. In some embodiments, the composition comprises about 40.8% of the PEG 300. In some embodiments, the composition comprises about 40.78% of the PEG 300.

In some embodiments, the composition comprises about 6% to about 9% of the water, for example, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, or about 9.0% of the water.

In some embodiments, the composition comprises about 6% to about 8% of the water. In some embodiments, the composition comprises about 7% to about 8% of the water. In some embodiments, the composition comprises about 7% of the water. In some embodiments, the composition comprises about 7% to about 7.5% of the water. In some embodiments, the composition comprises about 7.1% to about 7.3% of the water. In some embodiments, the composition comprises about 7.2% of the water. In some embodiments, the composition comprises about 7.20% of the water.

In some embodiments, the composition comprises about 7% to about 12% of the ethanol, for example, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.1%, about 10.2%, about 10.3%, about 10.4%, about 10.5%, about 10.6%, about 10.7%, about 10.8%, about 10.9%, about 11.1%, about 11.2%, about 11.3%, about 11.4%, about 11.5%, about 11.6%, about 11.7%, about 11.8%, about 11.9%, or about 12% of the ethanol.

In some embodiments, the composition comprises about 9% to about 11% of the ethanol. In some embodiments, the composition comprises about 9.75% to about 10.25% of the ethanol. In some embodiments, the composition comprises about 9.9% to about 10.1% of the ethanol. In some embodiments, the composition comprises about 10% of the ethanol. In some embodiments, the composition comprises about 10.0% of the ethanol.

In some embodiments, the composition comprises:
about 40% to about 43% of the free acid form of the compound of Formula Ia or Formula Ib;
about 40% to about 42% of the PEG 300;
about 9% to about 11% of the ethanol; and
about 6% to about 8% of the water.

In some embodiments, the composition comprises:
about 42% to about 43% of the free acid form of the compound of Formula Ia or Formula Ib;
about 40% to about 41% of the PEG 300;
about 9% to about 11% of the ethanol; and
about 7% to about 8% of the water.

In some embodiments, the composition comprises:
about 42.0% to about 42.1% of the free acid form of the compound of Formula Ia or Formula Ib;
about 40.7% to about 40.8% of the PEG 300;
about 9.9% to about 10.1% of the ethanol; and
about 7.1% to about 7.3% of the water.

In some embodiments, the composition comprises:
about 42% of the free acid form of the compound of Formula Ia or Formula Ib;
about 41% of the PEG 300;
about 10% of the ethanol; and
about 7% of the water.

In some embodiments, the composition comprises:
about 42.02% of the free acid form of the compound of Formula Ia or Formula Ib;
about 40.78% of the PEG 300;
about 10% of the ethanol; and
about 7.20% of the water.

In some embodiments, the composition consists of:
about 40% to about 43% of the free acid form of the compound of Formula Ia or Formula Ib;
about 40% to about 42% of the PEG 300;
about 9% to about 11% of the ethanol; and
about 6% to about 8% of the water.

In some embodiments, the composition consists of:
about 42% to about 43% of the free acid form of the compound of Formula Ia or Formula Ib;
about 40% to about 41% of the PEG 300;
about 9% to about 11% of the ethanol; and
about 7% to about 8% of the water.

In some embodiments, the composition consists of:
about 42.0% to about 42.1% of the free acid form of the compound of Formula Ia or Formula Ib;
about 40.7% to about 40.8% of the PEG 300;
about 9.9% to about 10.1% of the ethanol; and
about 7.1% to about 7.3% of the water.

In some embodiments, the composition consists of:
about 42% of the free acid form of the compound of Formula Ia or Formula Ib; about 41% of the PEG 300;
about 10% of the ethanol; and
about 7% of the water.

In some embodiments, the composition consists of:
about 42.02% of the free acid form of the compound of Formula Ia or Formula Ib;
about 40.78% of the PEG 300;
about 10% of the ethanol; and
about 7.20% of the water.

### Composition 5

In some embodiments, the present disclosure relates to a pharmaceutical composition comprising a compound of Formula Ia:

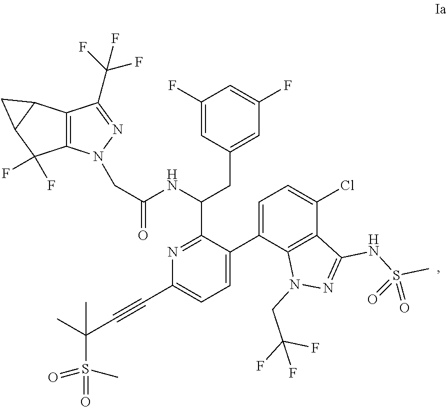 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, PEG 300, poloxamer 188, and water, wherein the composition comprises:
about 30% to about 40% of the compound of Formula Ia, or a pharmaceutically acceptable salt thereof;
about 35% to about 45% of the PEG 300;
about 5% to about 10% poloxamer 188; and
about 15% to about 20% of the water.

In some embodiments, the present disclosure relates to a pharmaceutical composition comprising a compound of Formula Ib:

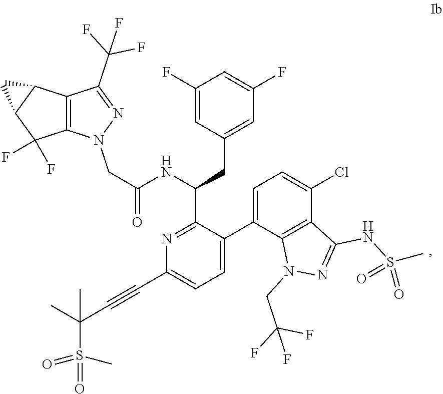 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, PEG 300, water, and ethanol, wherein the composition comprises:
about 30% to about 40% of the compound of Formula Ib, or a pharmaceutically acceptable salt thereof;
about 35% to about 45% of the PEG 300;
about 5% to about 10% poloxamer 188; and
about 15% to about 20% of the water.

In some embodiments, the composition comprises about 30% to about 35% of the compound of Formula Ia or Formula Ib, or a pharmaceutically acceptable salt thereof, for example, about 30.0%, about 30.1%, about 30.2%, about 30.3%, about 30.4%, about 30.5%, about 30.6%, about 30.7%, about 30.8%, about 30.9%, about 31.0%, about 31.1%, about 31.2%, about 31.3%, about 31.4%, about 31.5%, about 31.6%, about 31.7%, about 31.8%, about 31.9%, about 32.0%, about 32.1%, about 32.2%, about 32.3%, about 32.4%, about 32.5%, about 32.6%, about 32.7%, about 32.8%, about 32.9%, 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, or about 35.0% of the compound of Formula Ia or Formula Ib, or a pharmaceutically acceptable salt thereof.

In some embodiments, the composition comprises about 32% to about 34% of the compound of Formula Ia or Formula Ib, or a pharmaceutically acceptable salt thereof. In some embodiments, the composition comprises about 33% to about 34% of the compound of Formula Ia or Formula Ib, or a pharmaceutically acceptable salt thereof. In some embodiments, the composition comprises about 33% of the compound of Formula Ia or Formula Ib, or a pharmaceutically acceptable salt thereof. In some embodiments, the composition comprises about 34% of the compound of Formula Ia or Formula Ib, or a pharmaceutically acceptable salt thereof. In some embodiments, the composition comprises about 33.6% of the compound of Formula Ia or Formula Ib, or a pharmaceutically acceptable salt thereof. In some embodiments, the composition comprises about 33.1% of the compound of Formula Ia or Formula Ib, or a pharmaceutically acceptable salt thereof.

In some embodiments, the composition comprises about 32% to about 34% of a sodium salt of the compound of Formula Ia or Formula Ib. In some embodiments, the composition comprises about 33% to about 34% of a sodium salt of the compound of Formula Ia or Formula Ib. In some embodiments, the composition comprises about 33% of a sodium salt of the compound of Formula Ia or Formula Ib. In some embodiments, the composition comprises about 34% of a sodium salt of the compound of Formula Ia or Formula Ib. In some embodiments, the composition comprises about 33.6% of a sodium salt of the compound of Formula Ia or Formula Ib. In some embodiments, the composition comprises about 33.1% of a sodium salt of the compound of Formula Ia or Formula Ib.

In some embodiments, the composition comprises about 40% to about 43% of the PEG 300, for example, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, or about 43.0% of the PEG 300.

In some embodiments, the composition comprises about 41% to about 42% of the PEG 300. In some embodiments, the composition comprises about 41.0% to about 41.1% of the PEG 300. In some embodiments, the composition comprises about 41.0% of the PEG 300. In some embodiments, the composition comprises about 41.1% of the PEG 300. In some embodiments, the composition comprises about 41.09% of the PEG 300.

In some embodiments, the composition comprises about 5% to about 7% of the poloxamer 188, for example, about 5.0%, about 5.1%, about 5.2%, about 5.3%, about 5.4%, about 5.5%, about 5.6%, about 5.7%, about 5.8%, about 5.9%, about 6.0%, about 6.0%, about 6.1%, about 6.2%, about 6.3%, about 6.4%, about 6.5%, about 6.6%, about 6.7%, about 6.8%, about 6.9%, or about 7.0% of the poloxamer 188.

In some embodiments, the composition comprises about 6.0% to about 7.0% of the poloxamer 188. In some embodiments, the composition comprises about 6.1% to about 6.2% of the poloxamer 188. In some embodiments, the composition comprises about 6.1% of the poloxamer 188. In some embodiments, the composition comprises about 6.2% of the poloxamer 188. In some embodiments, the composition comprises about 6.12% of the poloxamer 188.

In some embodiments, the composition comprises about 15% to about 20% of the water, for example, about 15.0%, about 15.1%, about 15.2%, about 15.3%, about 15.4%, about 15.5%, about 15.6%, about 15.7%, about 15.8%, about 15.9%, about 16.0%, about 16.1%, about 16.2%, about 16.3%, about 16.4%, about 16.5%, about 16.6%, about 16.7%, about 16.8%, about 16.9%, about 17.0%, about 17.1%, about 17.2%, about 17.3%, about 17.4%, about 17.5%, about 17.6%, about 17.7%, about 17.8%, about 17.9%, about 18.0%, about 18.1%, about 18.2%, about 18.3%, about 18.4%, about 18.5%, about 18.6%, about 18.7%, about 18.8%, about 18.9%, about 19.0%, about 19.1%, about 19.2%, about 19.3%, about 19.4%, about 19.5%, about 19.6%, about 19.7%, about 19.8%, about 19.9%, or about 20.0% of the water.

In some embodiments, the composition comprises about 19% to about 20% of the water. In some embodiments, the composition comprises about 19.1% to about 19.3% of the water. In some embodiments, the composition comprises about 19.1% of the water. In some embodiments, the composition comprises about 19.2% of the water. In some embodiments, the composition comprises about 19.18% of the water.

In some embodiments, the composition comprises:
about 32% to about 34% of the compound of Formula Ia or Formula Ib, or a pharmaceutically acceptable salt thereof;
about 40% to about 43% of the PEG 300;
about 5% to about 7% poloxamer 188; and
about 19% to about 20% of the water.

In some embodiments, the composition comprises:
about 33% of the compound of Formula Ia or Formula Ib, or a pharmaceutically acceptable salt thereof;
about 41% of the PEG 300;
about 6% poloxamer 188; and
about 19% of the water.

In some embodiments, the composition comprises:
about 33.6% of the compound of Formula Ia or Formula Ib, or a pharmaceutically acceptable salt thereof;
about 41.1% of the PEG 300;
about 6.1% poloxamer 188; and
about 19.2% of the water.

In some embodiments, the composition comprises:
about 33.61% of the compound of Formula Ia or Formula Ib, or a pharmaceutically acceptable salt thereof;
about 41.09% of the PEG 300;
about 6.12% poloxamer 188; and
about 19.18% of the water.

In some embodiments, the composition comprises:
about 32% to about 34% of a sodium salt of the compound of Formula Ia or Formula Ib;
about 40% to about 43% of the PEG 300;
about 5% to about 7% poloxamer 188; and
about 19% to about 20% of the water.

In some embodiments, the composition comprises:
about 33% of a sodium salt of the compound of Formula Ia or Formula Ib;
about 41% of the PEG 300;
about 6% poloxamer 188; and
about 19% of the water.

In some embodiments, the composition comprises:
about 33.6% of a sodium salt of the compound of Formula Ia or Formula Ib;
about 41.1% of the PEG 300;
about 6.1% poloxamer 188; and
about 19.2% of the water.

In some embodiments, the composition comprises:
about 33.61% of a sodium salt of the compound of Formula Ia or Formula Ib;
about 41.09% of the PEG 300;
about 6.12% poloxamer 188; and
about 19.18% of the water.

In some embodiments, the composition consists of:
about 32% to about 34% of the compound of Formula Ia or Formula Ib, or a pharmaceutically acceptable salt thereof;
about 40% to about 43% of the PEG 300;
about 5% to about 7% poloxamer 188; and
about 19% to about 20% of the water.

In some embodiments, the composition consists of:
about 33% of the compound of Formula Ia or Formula Ib, or a pharmaceutically acceptable salt thereof;
about 41% of the PEG 300;
about 6% poloxamer 188; and
about 19% of the water.

In some embodiments, the composition consists of:
about 33.6% of the compound of Formula Ia or Formula Ib, or a pharmaceutically acceptable salt thereof;
about 41.1% of the PEG 300;
about 6.1% poloxamer 188; and
about 19.2% of the water.

In some embodiments, the composition consists of:
about 33.61% of the compound of Formula Ia or Formula Ib, or a pharmaceutically acceptable salt thereof;
about 41.09% of the PEG 300;
about 6.12% poloxamer 188; and
about 19.18% of the water.

In some embodiments, the composition consists of:
about 32% to about 34% of a sodium salt of the compound of Formula Ia or Formula Ib;
about 40% to about 43% of the PEG 300;
about 5% to about 7% poloxamer 188; and
about 19% to about 20% of the water.

In some embodiments, the composition consists of:
about 33% of a sodium salt of the compound of Formula Ia or Formula Ib; about 41% of the PEG 300;
about 6% poloxamer 188; and
about 19% of the water.

In some embodiments, the composition consists of:
about 33.6% of a sodium salt of the compound of Formula Ia or Formula Ib; about 41.1% of the PEG 300;
about 6.1% poloxamer 188; and
about 19.2% of the water.

In some embodiments, the composition consists of:
about 33.61% of a sodium salt of the compound of Formula Ia or Formula Ib; about 41.09% of the PEG 300;
about 6.12% poloxamer 188; and
about 19.18% of the water.

### Composition 6

In some embodiments, the present disclosure relates to a pharmaceutical composition comprising a compound of Formula Ia:

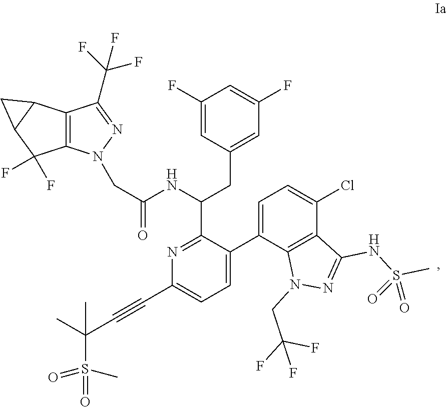 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 30% to about 45% of the free acid form of the compound of Formula Ia;
about 40% to about 50% of the PEG 300;
about 5% to about 10% of the water; and
about 7% to about 15% of the ethanol.

In some embodiments, the present disclosure relates to a pharmaceutical composition comprising a compound of Formula Ib:

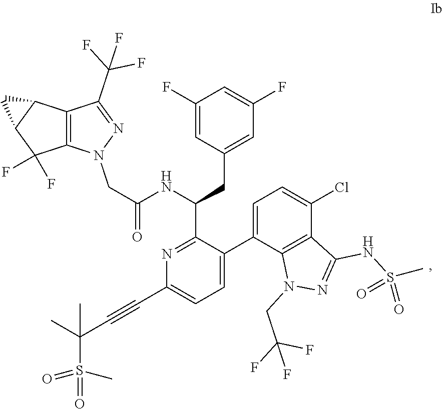 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 30% to about 45% of the free acid form of the compound of Formula Ib;
about 40% to about 50% of the PEG 300;
about 5% to about 10% of the water; and
about 7% to about 15% of the ethanol.

In some embodiments, the composition comprises about 33% to about 36% of the free acid form of the compound of Formula Ia or Formula Ib, for example, about 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, about 35.0%, about 35.1%, about 35.2%, about 35.3%, about 35.4%, about 35.5%, about 35.6%, about 35.7%, about 35.8%, about 35.9%, or about 36.0%, of the free acid form of the compound of Formula Ia or Formula Ib.

In some embodiments, the composition comprises about 34% to about 35% of the free acid form of the compound of Formula Ia or Formula Ib. In some embodiments, the composition comprises about 34% of the free acid form of the compound of Formula Ia or Formula Ib. In some embodiments, the composition comprises about 34.1% to about 34.2% of the free acid form of the compound of Formula Ia or Formula Ib. In some embodiments, the composition comprises about 34.1% of the free acid form of the compound of Formula Ia or Formula Ib. In some embodiments, the composition comprises about 34.2% of the free acid form of the compound of Formula Ia or Formula Ib. In some embodiments, the composition comprises about 34.19% of the free acid form of the compound of Formula Ia or Formula Ib.

In some embodiments, the composition comprises about 45% to about 50% of the PEG 300, for example, about 45.0%, about 45.1%, about 45.2%, about 45.3%, about 45.4%, about 45.5%, about 45.6%, about 45.7%, about 45.8%, about 45.9%, about 46.0%, about 46.1%, about 46.2%, about 46.3%, about 46.4%, about 46.5%, about 46.6%, about 46.7%, about 46.8%, about 46.9%, about 47.0%, about 47.1%, about 47.2%, about 47.3%, about 47.4%, about 47.5%, about 47.6%, about 47.7%, about 47.8%, about 47.9%, about 48.0%, about 48.1%, about 48.2%, about 48.3%, about 48.4%, about 48.5%, about 48.6%, about 48.7%, about 48.8%, about 48.9%, about 49.0%, about 49.1%, about 49.2%, about 49.3%, about 49.4%, about 49.5%, about 49.6%, about 49.7%, about 49.8%, about 49.9%, or about 50.0% of the PEG 300.

In some embodiments, the composition comprises about 47% to about 48% of the PEG 300. In some embodiments, the composition comprises about 47% of the PEG 300. In some embodiments, the composition comprises about 47.4% to about 47.5% of the PEG 300. In some embodiments, the composition comprises about 47.4% of the PEG 300. In some embodiments, the composition comprises about 47.44% of the PEG 300.

In some embodiments, the composition comprises about 8% to about 9% of the water, for example, about 8.0%, about 8.1%, about 8.2%, about 8.3%, about 8.4%, about 8.5%, about 8.6%, about 8.7%, about 8.8%, about 8.9%, or about 9.0% of the water.

In some embodiments, the composition comprises about 8% of the water.

In some embodiments, the composition comprises about 8.3% to about 8.4% of the water. In some embodiments, the composition comprises about 8.3% of the water. In some embodiments, the composition comprises about 8.4% of the water. In some embodiments, the composition comprises about 8.37% of the water.

In some embodiments, the composition comprises about 7% to about 12% of the ethanol, for example, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.1%, about 10.2%, about 10.3%, about 10.4%, about 10.5%, about 10.6%, about 10.7%, about 10.8%, about 10.9%, about 11.1%, about 11.2%, about 11.3%, about 11.4%, about 11.5%, about 11.6%, about 11.7%, about 11.8%, about 11.9%, or about 12% of the ethanol.

In some embodiments, the composition comprises about 9% to about 11% of the ethanol. In some embodiments, the composition comprises about 9.75% to about 10.25% of the ethanol. In some embodiments, the composition comprises about 9.9% to about 10.1% of the ethanol. In some embodiments, the composition comprises about 10% of the ethanol. In some embodiments, the composition comprises about 10.0% of the ethanol.

In some embodiments, the composition comprises:
about 33% to about 36% of the free acid form of the compound of Formula Ia or Formula Ib;
about 45% to about 50% of the PEG 300;
about 6% to about 9% of the water; and
about 7% to about 12% of the ethanol.

In some embodiments, the composition comprises:
about 34% to about 35% of the free acid form of the compound of Formula Ia or Formula Ib;
about 47% to about 48% of the PEG 300;
about 8% to about 9% of the water; and
about 9% to about 11% of the ethanol.

In some embodiments, the composition comprises:
about 34.1% to about 34.2% of the free acid form of the compound of Formula Ia or Formula Ib;
about 47.4% to about 47.5% of the PEG 300;
about 8.3% to about 8.4% of the water; and
about 10% of the ethanol.

In some embodiments, the composition comprises:
about 34.19% of the free acid form of the compound of Formula Ia or Formula Ib;
about 47.44% of the PEG 300;
about 8.37% of the water; and
about 10% of the ethanol.

In some embodiments, the composition consists of:
about 33% to about 36% of the free acid form of the compound of Formula Ia or Formula Ib;
about 45% to about 50% of the PEG 300;
about 6% to about 9% of the water; and
about 7% to about 12% of the ethanol.

In some embodiments, the composition consists of:
about 34% to about 35% of the free acid form of the compound of Formula Ia or Formula Ib;
about 47% to about 48% of the PEG 300;
about 8% to about 9% of the water; and
about 9% to about 11% of the ethanol.

In some embodiments, the composition consists of:
about 34.1% to about 34.2% of the free acid form of the compound of Formula Ia or Formula Ib;
about 47.4% to about 47.5% of the PEG 300;
about 8.3% to about 8.4% of the water; and
about 10% of the ethanol.

In some embodiments, the composition consists of:
about 34.19% of the free acid form of the compound of Formula Ia or Formula Ib; about 47.44% of the PEG 300;
about 8.37% of the water; and
about 10% of the ethanol.

### Composition 7

In some embodiments, the present disclosure relates to a pharmaceutical composition comprising a compound of Formula Ia:

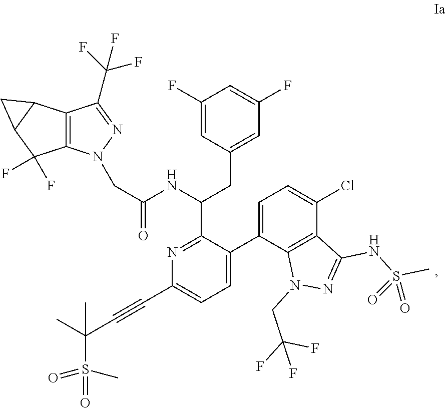 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, water, and ethanol, wherein the composition comprises:
about 30% to about 40% of the free acid form of the compound of Formula Ia;
about 45% to about 55% of the PEG 300;
about 5% to about 10% of the water; and
about 2% to about 7% of the ethanol.

In some embodiments, the present disclosure relates to a pharmaceutical composition comprising a compound of Formula Ib:

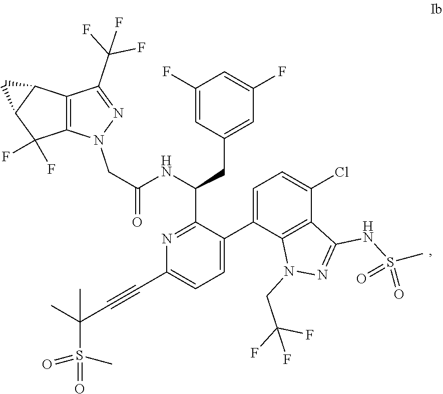 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, water, and ethanol, wherein the composition comprises:
about 30% to about 40% of the free acid form of the compound of Formula Ib;
about 45% to about 55% of the PEG 300;
about 5% to about 10% of the water; and
about 2% to about 7% of the ethanol.

In some embodiments, about 33% to about 36% of the free acid form of the compound of Formula Ia or Formula Ib, for example, about 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, about 35.0%, about 35.1%, about 35.2%, about 35.3%, about 35.4%, about 35.5%, about 35.6%, about 35.7%, about 35.8%, about 35.9%, or about 36.0%, of the free acid form of the compound of Formula Ia or Formula Ib.

In some embodiments, the composition comprises about 33% to about 34% of the free acid form of the compound of Formula Ia or Formula Ib. In some embodiments, the composition comprises about 34% of the free acid form of the compound of Formula Ia or Formula Ib. In some embodiments, the composition comprises about 33.7% to about 33.8% of the free acid form of the compound of Formula Ia or Formula Ib. In some embodiments, the composition comprises about 33.7% of the free acid form of the compound of Formula Ia or Formula Ib. In some embodiments, the composition comprises about 33.8% of the free acid form of the compound of Formula Ia or Formula Ib. In some embodiments, the composition comprises about 33.78% of the free acid form of the compound of Formula Ia or Formula Ib.

In some embodiments, the composition comprises about 50% to about 55% of the PEG 300, for example, about 50.1%, about 50.2%, about 50.3%, about 50.4%, about 50.5%, about 50.6%, about 50.7%, about 50.8%, about 50.9%, about 51.0%, about 51.1%, about 51.2%, about 51.3%, about 51.4%, about 51.5%, about 51.6%, about 51.7%, about 51.8%, about 51.9%, about 52.0%, about 52.1%, about 52.2%, about 52.3%, about 52.4%, about 52.5%, about 52.6%, about 52.7%, about 52.8%, about 52.9%, about 53.0%, about 53.1%, about 53.2%, about 53.3%, about 53.4%, about 53.5%, about 53.6%, about 53.7%, about 53.8%, about 53.9%, about 54.0%, about 54.1%, about 54.2%, about 54.3%, about 54.4%, about 54.5%, about 54.6%, about 54.7%, about 54.8%, about 54.9%, or about 55.0% of the PEG 300.

In some embodiments, the composition comprises about 52% to about 53% of the PEG 300. In some embodiments, the composition comprises about 52% of the PEG 300. In some embodiments, the composition comprises about 52.0% to about 52.1% of the PEG 300. In some embodiments, the composition comprises about 52.0% of the PEG 300. In some embodiments, the composition comprises about 52.02% of the PEG 300.

In some embodiments, the composition comprises about 7% to about 9% of the water, for example, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, or about 9.0% of the water.

In some embodiments, the composition comprises about 9% to about 10% % of the water. In some embodiments, the composition comprises about 9.1% to about 9.2% of the water. In some embodiments, the composition comprises about 9.1% of the water. In some embodiments, the composition comprises about 9.2% of the water. In some embodiments, the composition comprises about 9.18% of the water.

In some embodiments, the composition comprises about 4% to about 6% of the ethanol, for example, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, or about 6.0% of the ethanol.

In some embodiments, the composition comprises about 4.5% to about 5.5% of the ethanol. In some embodiments, the composition comprises about 4.9% to about 5.1% of the ethanol. In some embodiments, the composition comprises about 5.0% of the ethanol. In some embodiments, the composition comprises about 5.00% of the ethanol.

In some embodiments, the composition comprises:
about 33% to about 34% of the free acid form of the compound of Formula Ia or Formula Ib;
about 52% to about 53% of the PEG 300;
about 7% to about 9% of the water; and
about 4% to about 6% of the ethanol.

In some embodiments, the composition comprises:
about 34% of the free acid form of the compound of Formula Ia or Formula Ib; about 52% of the PEG 300;
about 9% of the water; and
about 5% of the ethanol.

In some embodiments, the composition comprises:
about 33.8% of the free acid form of the compound of Formula Ia or Formula Ib;
about 52.0% of the PEG 300;
about 9.2% of the water; and
about 5.0% of the ethanol.

In some embodiments, the composition comprises:
about 33.78% of the free acid form of the compound of Formula Ia or Formula Ib;
about 52.02% of the PEG 300;
about 9.18% of the water; and
about 5.00% of the ethanol.

In some embodiments, the composition consists of:
about 33% to about 34% of the free acid form of the compound of Formula Ia or Formula Ib;
about 52% to about 53% of the PEG 300;
about 7% to about 9% of the water; and
about 4% to about 6% of the ethanol.

In some embodiments, the composition consists of:
about 34% of the free acid form of the compound of Formula Ia or Formula Ib; about 52% of the PEG 300;
about 9% of the water; and
about 5% of the ethanol.

In some embodiments, the composition consists of:
about 33.8% of the free acid form of the compound of Formula Ia or Formula Ib; about 52.0% of the PEG 300;
about 9.2% of the water; and
about 5.0% of the ethanol.

In some embodiments, the composition consists of:
about 33.78% of the free acid form of the compound of Formula Ia or Formula Ib; about 52.02% of the PEG 300;
about 9.18% of the water; and
about 5.00% of the ethanol.

The pharmaceutical compositions disclosed herein can be prepared by methodologies known in the pharmaceutical art. For example, in certain embodiments, a pharmaceutical composition intended to be administered by injection (e.g., intramuscular or subcutaneous administration) can prepared by combining the compound of Formula Ia or Ib, or a pharmaceutically acceptable salt or free acid form thereof, with sterile, distilled water so as to form a solution. In some embodiments, the compositions provided herein are solutions. In some embodiments, a surfactant is added to facilitate the formation of a homogeneous solution or suspension. Surfactants are compounds that non-covalently interact with the compound of Formula Ia or Ib, or a pharmaceutically acceptable salt or free acid form thereof, so as to facilitate dissolution or homogeneous suspension of the compound in the aqueous delivery system.

The terms “effective amount” or “therapeutically effective amount” refer to an amount of the compound of Formula Ia or Ib, or a pharmaceutically acceptable salt or free acid form thereof, which when administered to a patient in need thereof, is sufficient to effect treating or preventing an HIV infection or reducing the risk of contracting HIV infection, as described herein. Such an amount would be sufficient to elicit the biological or medical response of a tissue system, or patient that is sought by a researcher or clinician. The amount of the compound of Formula Ia or Ib which constitutes a therapeutically effective amount will vary depending on such factors as the compound, salt, free acid, or composition used for administration, the time of administration, the route of administration, the rate of excretion of the compound, the duration of the treatment, the type of disease-state or disorder being treated and its severity, and the age, body weight, general health, sex and diet of the patient. Such a therapeutically effective amount can be determined routinely by one of ordinary skill in the art having regard to their own knowledge, the state of the art, and this disclosure.

### Methods of Use

In some embodiments, the methods provided herein comprise treating or preventing a human immunodeficiency virus (HIV) infection in the patient. In some embodiments, the methods provided herein comprise treating a HIV infection in the patient. In some embodiments, the methods provided herein comprise preventing a HIV infection in the patient. In some embodiments, the patient may have or be at risk of contracting an HIV infection. In some embodiments, the patient has been identified as an individual who is at risk of sexual transmission of HIV. In some embodiments, the individual has been identified as a man (e.g., who has sexual intercourse with a man or a woman), transgender man, transgender woman, a woman (e.g., who has sexual intercourse with a man or a woman), as a gender non-binary individual (e.g., who has sexual intercourse with a man or a woman), and/or a sex worker.

In some embodiments, the individual has been identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual has been identified as having sex with partners of unknown HIV status.

In some embodiments, the individual has been identified as having sex with people who are living with HIV but not on HIV treatment and with an undetectable viral load.

In some embodiments, the individual has been identified as having sex in a geographic area where HIV is common.

In some embodiments, the individual has been identified as having sex in a geographic area of North America (e.g., United States, Canada, Mexico) where HIV is common. In some embodiments, the individual has been identified as having sex in a geographic area of the United States where HIV is common. In some embodiments, the individual has been identified as having sex in a geographic area of the Canada where HIV is common. In some embodiments, the individual has been identified as having sex in a geographic area of the Mexico where HIV is common.

In some embodiments, the individual has been identified as having sex in a geographic area of Africa where HIV is common. In some embodiments, the individual has been identified as having sex in a geographic area of South Africa where HIV is common. In some embodiments, the individual has been identified as having sex in a geographic area of Uganda where HIV is common.

In some embodiments, the individual has been identified as having sex in a geographic area of Europe where HIV is common.

In some embodiments, the individual has been identified as having sex in a geographic area of Asia (e.g., Thailand) where HIV is common. In some embodiments, the individual has been identified as having sex in a geographic area of Thailand where HIV is common.

In some embodiments, the individual has been identified as having sex in a geographic area of South America (e.g., Argentina, Brazil, Peru, and the like) where HIV is common. In some embodiments, the individual has been identified as having sex in a geographic area of Argentina where HIV is common. In some embodiments, the individual has been identified as having sex in a geographic area of Brazil where HIV is common. In some embodiments, the individual has been identified as having sex in a geographic area of Peru where HIV is common.

In some embodiments, the individual has been identified as having sex while under the influence of substances or alcohol.

In some embodiments, the individual has been identified as not using condoms consistently with partners of unknown status.

In some embodiments, the individual has been identified an individual who injects drugs.

In some embodiments, the individual is a cisgender woman. In some embodiments, the individual is an adolescent cisgender woman. In some embodiments, the adolescent cisgender woman is about 16 to about 25 years of age, for example, about 16, about 17, about 18, about 19, about 20, about 21, about 22, about 23, about 24, about 25, or about 26 years of age.

In some embodiments, the individual is a cisgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender woman identified as having sex with partners of unknown HIV status.

In some embodiments, the individual is a cisgender woman identified as having sex with people who are living with HIV but not on HIV treatment and with an undetectable viral load.

In some embodiments, the individual is a cisgender woman identified as having sex in a geographic area where HIV is common.

In some embodiments, the individual is a cisgender woman identified as having sex in a geographic area of North America (e.g., United States, Canada, Mexico) where HIV is common. In some embodiments, the individual is a cisgender woman identified as having sex in a geographic area of the United States where HIV is common. In some embodiments, the individual is a cisgender woman identified as having sex in a geographic area of the Canada where HIV is common. In some embodiments, the individual is a cisgender woman identified as having sex in a geographic area of the Mexico where HIV is common.

In some embodiments, the individual is a cisgender woman identified as having sex in a geographic area of Africa where HIV is common. In some embodiments, the individual is a cisgender woman identified as having sex in a geographic area of South Africa where HIV is common. In some embodiments, the individual is a cisgender woman identified as having sex in a geographic area of Uganda where HIV is common.

In some embodiments, the individual is a cisgender woman identified as having sex in a geographic area of Europe where HIV is common.

In some embodiments, the individual is a cisgender woman identified as having sex in a geographic area of Asia (e.g., Thailand) where HIV is common. In some embodiments, the individual is a cisgender woman identified as having sex in a geographic area of Thailand where HIV is common.

In some embodiments, the individual is a cisgender woman identified as having sex in a geographic area of South America (e.g., Argentina, Brazil, Peru, and the like) where HIV is common. In some embodiments, the individual is a cisgender woman identified as having sex in a geographic area of Argentina where HIV is common. In some embodiments, the individual is a cisgender woman identified as having sex in a geographic area of Brazil where HIV is common. In some embodiments, the individual is a cisgender woman identified as having sex in a geographic area of Peru where HIV is common.

In some embodiments, the individual is a cisgender woman identified as having sex while under the influence of substances or alcohol.

In some embodiments, the individual is a cisgender woman identified as not using condoms consistently with partners of unknown status.

In some embodiments, the individual is a cisgender woman identified as an individual who injects drugs.

In some embodiments, the individual is a transgender woman. In some embodiments, the individual is an adolescent transgender woman. In some embodiments, the adolescent transgender woman is about 16 to about 25 years of age, for example, about 16, about 17, about 18, about 19, about 20, about 21, about 22, about 23, about 24, about 25, or about 26 years of age.

In some embodiments, the individual is a transgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender woman identified as having sex with partners of unknown HIV status.

In some embodiments, the individual is a transgender woman identified as having sex with people who are living with HIV but not on HIV treatment and with an undetectable viral load.

In some embodiments, the individual is a transgender woman identified as having sex in a geographic area where HIV is common.

In some embodiments, the individual is a transgender woman identified as having sex in a geographic area of North America (e.g., United States, Canada, Mexico) where HIV is common. In some embodiments, the individual is a transgender woman identified as having sex in a geographic area of the United States where HIV is common. In some embodiments, the individual is a transgender woman identified as having sex in a geographic area of the Canada where HIV is common. In some embodiments, the individual is a transgender woman identified as having sex in a geographic area of the Mexico where HIV is common.

In some embodiments, the individual is a transgender woman identified as having sex in a geographic area of Africa where HIV is common. In some embodiments, the individual is a transgender woman identified as having sex in a geographic area of South Africa where HIV is common. In some embodiments, the individual is a transgender woman identified as having sex in a geographic area of Uganda where HIV is common.

In some embodiments, the individual is a transgender woman identified as having sex in a geographic area of Europe where HIV is common.

In some embodiments, the individual is a transgender woman identified as having sex in a geographic area of Asia (e.g., Thailand) where HIV is common. In some embodiments, the individual is a transgender woman identified as having sex in a geographic area of Thailand where HIV is common.

In some embodiments, the individual is a transgender woman identified as having sex in a geographic area of South America (e.g., Argentina, Brazil, Peru, and the like) where HIV is common. In some embodiments, the individual is a transgender woman identified as having sex in a geographic area of Argentina where HIV is common. In some embodiments, the individual is a transgender woman identified as having sex in a geographic area of Brazil where HIV is common. In some embodiments, the individual is a transgender woman identified as having sex in a geographic area of Peru where HIV is common.

In some embodiments, the individual is a transgender woman identified as having sex while under the influence of substances or alcohol.

In some embodiments, the individual is a transgender woman identified as not using condoms consistently with partners of unknown status.

In some embodiments, the individual is a transgender woman identified as an individual who injects drugs.

In some embodiments, the individual is a cisgender man. In some embodiments, the individual is an adolescent cisgender man. In some embodiments, the adolescent cisgender man is about 16 to about 25 years of age, for example, about 16, about 17, about 18, about 19, about 20, about 21, about 22, about 23, about 24, about 25, or about 26 years of age.

In some embodiments, the individual is a cisgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender man identified as having sex with partners of unknown HIV status.

In some embodiments, the individual is a cisgender man identified as having sex with people who are living with HIV but not on HIV treatment and with an undetectable viral load.

In some embodiments, the individual is a cisgender man identified as having sex in a geographic area where HIV is common.

In some embodiments, the individual is a cisgender man identified as having sex in a geographic area of North America (e.g., United States, Canada, Mexico) where HIV is common.

In some embodiments, the individual is a cisgender man identified as having sex in a geographic area of the United States where HIV is common. In some embodiments, the individual is a cisgender man identified as having sex in a geographic area of the Canada where HIV is common. In some embodiments, the individual is a cisgender man identified as having sex in a geographic area of the Mexico where HIV is common.

In some embodiments, the individual is a cisgender man identified as having sex in a geographic area of Africa where HIV is common. In some embodiments, the individual is a cisgender man identified as having sex in a geographic area of South Africa where HIV is common. In some embodiments, the individual is a cisgender man identified as having sex in a geographic area of Uganda where HIV is common.

In some embodiments, the individual is a cisgender man identified as having sex in a geographic area of Europe where HIV is common.

In some embodiments, the individual is a cisgender man identified as having sex in a geographic area of Asia (e.g., Thailand) where HIV is common. In some embodiments, the individual is a cisgender man identified as having sex in a geographic area of Thailand where HIV is common.

In some embodiments, the individual is a cisgender man identified as having sex in a geographic area of South America (e.g., Argentina, Brazil, Peru, and the like) where HIV is common. In some embodiments, the individual is a cisgender man identified as having sex in a geographic area of Argentina where HIV is common. In some embodiments, the individual is a cisgender man identified as having sex in a geographic area of Brazil where HIV is common. In some embodiments, the individual is a cisgender man identified as having sex in a geographic area of Peru where HIV is common.

In some embodiments, the individual is a cisgender man identified as having sex while under the influence of substances or alcohol.

In some embodiments, the individual is a cisgender man identified as not using condoms consistently with partners of unknown status.

In some embodiments, the individual is a cisgender man identified as an individual who injects drugs.

In some embodiments, the individual is a transgender man. In some embodiments, the individual is an adolescent transgender man. In some embodiments, the adolescent transgender man is about 16 to about 25 years of age, for example, about 16, about 17, about 18, about 19, about 20, about 21, about 22, about 23, about 24, about 25, or about 26 years of age.

In some embodiments, the individual is a transgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender man identified as having sex with partners of unknown HIV status.

In some embodiments, the individual is a transgender man identified as having sex with people who are living with HIV but not on HIV treatment and with an undetectable viral load.

In some embodiments, the individual is a transgender man identified as having sex in a geographic area where HIV is common.

In some embodiments, the individual is a transgender man identified as having sex in a geographic area of North America (e.g., United States, Canada, Mexico) where HIV is common. In some embodiments, the individual is a transgender man identified as having sex in a geographic area of the United States where HIV is common. In some embodiments, the individual is a transgender man identified as having sex in a geographic area of the Canada where HIV is common. In some embodiments, the individual is a transgender man identified as having sex in a geographic area of the Mexico where HIV is common.

In some embodiments, the individual is a transgender man identified as having sex in a geographic area of Africa where HIV is common. In some embodiments, the individual is a transgender man identified as having sex in a geographic area of South Africa where HIV is common. In some embodiments, the individual is a transgender man identified as having sex in a geographic area of Uganda where HIV is common.

In some embodiments, the individual is a transgender man identified as having sex in a geographic area of Europe where HIV is common.

In some embodiments, the individual is a transgender man identified as having sex in a geographic area of Asia (e.g., Thailand) where HIV is common. In some embodiments, the individual is a transgender man identified as having sex in a geographic area of Thailand where HIV is common.

In some embodiments, the individual is a transgender man identified as having sex in a geographic area of South America (e.g., Argentina, Brazil, Peru, and the like) where HIV is common. In some embodiments, the individual is a transgender man identified as having sex in a geographic area of Argentina where HIV is common. In some embodiments, the individual is a transgender man identified as having sex in a geographic area of Brazil where HIV is common. In some embodiments, the individual is a transgender man identified as having sex in a geographic area of Peru where HIV is common.

In some embodiments, the individual is a transgender man identified as having sex while under the influence of substances or alcohol.

In some embodiments, the individual is a transgender man identified as not using condoms consistently with partners of unknown status.

In some embodiments, the individual is a transgender man identified as an individual who injects drugs.

In some embodiments, the individual is a gender non-binary individual. In some embodiments, the individual is an adolescent gender non-binary individual. In some embodiments, the adolescent gender non-binary individual is about 16 to about 25 years of age, for example, about 16, about 17, about 18, about 19, about 20, about 21, about 22, about 23, about 24, about 25, or about 26 years of age.

In some embodiments, the individual is a gender non-binary individual identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a gender non-binary individual identified as having sex with partners of unknown HIV status.

In some embodiments, the individual is a gender non-binary individual identified as having sex with people who are living with HIV but not on HIV treatment and with an undetectable viral load.

In some embodiments, the individual is a gender non-binary individual identified as having sex in a geographic area where HIV is common.

In some embodiments, the individual is a gender non-binary individual identified as having sex in a geographic area of North America (e.g., United States, Canada, Mexico) where HIV is common. In some embodiments, the individual is a gender non-binary individual identified as having sex in a geographic area of the United States where HIV is common. In some embodiments, the individual is a gender non-binary individual identified as having sex in a geographic area of the Canada where HIV is common. In some embodiments, the individual is a gender non-binary individual identified as having sex in a geographic area of the Mexico where HIV is common.

In some embodiments, the individual is a gender non-binary individual identified as having sex in a geographic area of Africa where HIV is common. In some embodiments, the individual is a gender non-binary individual identified as having sex in a geographic area of South Africa where HIV is common. In some embodiments, the individual is a gender non-binary individual identified as having sex in a geographic area of Uganda where HIV is common.

In some embodiments, the individual is a gender non-binary individual identified as having sex in a geographic area of Europe where HIV is common.

In some embodiments, the individual is a gender non-binary individual identified as having sex in a geographic area of Asia (e.g., Thailand) where HIV is common. In some embodiments, the individual is a gender non-binary individual identified as having sex in a geographic area of Thailand where HIV is common.

In some embodiments, the individual is a gender non-binary individual identified as having sex in a geographic area of South America (e.g., Argentina, Brazil, Peru, and the like) where HIV is common. In some embodiments, the individual is a gender non-binary individual identified as having sex in a geographic area of Argentina where HIV is common. In some embodiments, the individual is a gender non-binary individual identified as having sex in a geographic area of Brazil where HIV is common. In some embodiments, the individual is a gender non-binary individual identified as having sex in a geographic area of Peru where HIV is common.

In some embodiments, the individual is a gender non-binary individual identified as having sex while under the influence of substances or alcohol.

In some embodiments, the individual is a gender non-binary individual identified as not using condoms consistently with partners of unknown status.

In some embodiments, the individual is a gender non-binary individual identified as an individual who injects drugs.

In some embodiments, the individual has been identified as:
having anal sex with at least two different sexual partners and no consistent condom use over the last 6 months; and/or
having history of sexually transmitted diseases (STDs) during the last 12 months (e.g., syphilis, gonorrhea, chlamydiae, HBV or HCV infection); and/or
using psycho-active drugs during sexual intercourses (e.g., cocaine, gammahydroxybutyric acid (GHB), methylenedioxymethamphetamine (MDMA), mephedrone); and/or
having sexual intercourse with one or more partners originating from a region with high prevalence of HIV infection (>1%) (e.g., South America, Sub-Saharan Africa, South-East Asia, Eastern Europe, French Guyana) and no consistent condom use; and/or
a sex worker; and/or
having a sexual partner who is an intravenous drug user sharing injection material; and/or
having an HIV-infected sexual partner with a detectable plasma viral load (e.g., >50 copies (cp)/milliliter (mL)); and/or
a cisgender man;
a transgender man;
a cisgender woman;
a transgender woman;
a gender non-binary individual; and/or
a person who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the patient is HIV-negative. In some embodiments, the HIV is HIV-1. In some embodiments, the HIV is HIV-2. In some embodiments, the HIV is HIV-1 and HIV-2.

As used herein, “HIV” or “Human Immunodeficiency Virus” refers to HIV-1 and/or to HIV-2.

In some embodiments, the patient is HIV-negative. In some embodiments, the patient is HIV-1 negative. In some embodiments, the patient is HIV-2 negative. In some embodiments, the patient is HIV-1 and HIV-2 negative.

In some embodiments, the patient has tested HIV-negative. In some embodiments, the patient has tested HIV-1 negative. In some embodiments, the patient has tested HIV-2 negative. In some embodiments, the patient has tested HIV-1 and HIV-2 negative.

In some embodiments, the patient has been identified as testing negative for HIV. In some embodiments, the patient has been identified as testing negative for HIV-1. In some embodiments, the patient has been identified as testing negative for HIV-2. In some embodiments, the patient has been identified as testing negative for HIV-1 and HIV-2.

In some embodiments, the method provided herein further comprises identifying the patient as testing negative for HIV. In some embodiments, the method provided herein further comprises identifying the patient as testing negative for HIV-1. In some embodiments, the method provided herein further comprises identifying the patient as testing negative for HIV-2. In some embodiments, the method provided herein further comprises identifying the patient as testing negative for HIV-1 and HIV-2.

In some embodiments, the method provided herein further comprises testing the patient for HIV prior to administration of the initiation dosage. In some embodiments, the method provided herein further comprises testing the patient for HIV-1 prior to administration of the initiation dosage. In some embodiments, the method provided herein further comprises testing the patient for HIV-2 prior to administration of the initiation dosage. In some embodiments, the method provided herein further comprises testing the patient for HIV-1 and HIV-2 prior to administration of the initiation dosage.

In some embodiments, the method provided herein further comprises testing the patient for HIV prior to administration of each administration of a composition provided herein (e.g., a composition comprising a free acid form of a compound of Formula Ia or Ib, PEG 300, ethanol, and water). In some embodiments, the method provided herein further comprises testing the patient for HIV-1 prior to each administration of a composition provided herein (e.g., a composition comprising a free acid form of a compound of Formula Ia or Ib, PEG 300, ethanol, and water). In some embodiments, the method provided herein further comprises testing the patient for HIV-2 prior to each administration of a composition provided herein (e.g., a composition comprising a free acid form of a compound of Formula Ia or Ib, PEG 300, ethanol, and water). In some embodiments, the method provided herein further comprises testing the patient for HIV-1 and HIV-2 prior to each administration of a composition provided herein (e.g., a composition comprising a free acid form of a compound of Formula Ia or Ib, PEG 300, ethanol, and water).

In some embodiments, the method provided herein comprises obtaining a negative test result of the patient for HIV, prior to administration of the initiation dosage. In some embodiments, the method provided herein comprises obtaining a negative test result of the patient for HIV-1, prior to administration of the initiation dosage. In some embodiments, the method provided herein comprises obtaining a negative test result of the patient for HIV-2, prior to administration of the initiation dosage. In some embodiments, the method provided herein comprises obtaining a negative test result of the patient for HIV-1 and HIV-2, prior to administration of the initiation dosage.

In some embodiments, the method provided herein comprises obtaining a negative test result for HIV of the patient, prior to each administration of a composition provided herein (e.g., a composition comprising a free acid form of a compound of Formula Ia or Ib, PEG 300, ethanol, and water). In some embodiments, the method provided herein comprises obtaining a negative test result of the patient for HIV-1, prior to each administration of a composition provided herein (e.g., a composition comprising a free acid form of a compound of Formula Ia or Ib, PEG 300, ethanol, and water). In some embodiments, the method provided herein comprises obtaining a negative test result of the patient for HIV-2, prior to each administration of a composition provided herein (e.g., a composition comprising a free acid form of a compound of Formula Ia or Ib, PEG 300, ethanol, and water). In some embodiments, the method provided herein comprises obtaining a negative test result of the patient for HIV-1 and HIV-2, prior to each administration of a composition provided herein (e.g., a composition comprising a free acid form of a compound of Formula Ia or Ib, PEG 300, ethanol, and water).

The term “patient” is meant to refer to a human who is in need of therapeutic or preventative treatment for a viral infection, such as HIV infection.

As used herein, the terms “prevention” or “preventing” refer to the administration of a compound, or a pharmaceutically acceptable salt or free acid form thereof, or composition comprising the compound, pharmaceutically acceptable salt, or free acid form thereof according to the present disclosure pre- or post-exposure of the patient to the virus but before the appearance of symptoms of the disease, and/or prior to the detection of the virus in the blood. The terms also refer to prevention of the appearance of symptoms of the disease and/or to prevent the virus from reaching detectable levels in the blood. The terms include both pre-exposure prophylaxis (PrEP), as well as post-exposure prophylaxis (PEP) and event driven or “on demand” prophylaxis. The terms also refer to prevention of perinatal transmission of HIV from mother to baby by administration of a compound, pharmaceutically acceptable salt thereof, or composition comprising the compound or the pharmaceutically acceptable salt thereof according to the present disclosure to the mother before giving birth and to the child within the first days of life. The terms also refers to prevention of transmission of HIV through blood transfusion.

As used herein the term “Ctau” refers to the observed drug concentration at the end of the dosing interval.

As used herein, the term “period of exposure” refers to a period of time, ranging from a single event or to multiple events over an extended period of time, in which a patient is exposed to HIV. For example, a patient who engages in one sexual intercourse event with a partner who is HIV-positive has a period of exposure that is limited to the time and duration of that one sexual intercourse event with that partner. As another example, a patient who has sexual intercourse with a partner who is HIV-positive on multiple occasions over an extended period of time (e.g., days, weeks, months, or years) has a period of exposure that ranges from the first instance to the last instance of sexual intercourse with that partner.

In some embodiments, the methods disclosed herein may comprise event driven administration of a composition provided herein (e.g., a composition comprising a compound of Formula Ia or Ib, or a pharmaceutically acceptable salt or free acid form thereof), to the patient. As used herein, the terms “event driven” or “event driven administration” refer to administration of a composition provided herein, (1) prior to an event (e.g., 2 hours, 1 day, 2 days, 5 days, 7 days, 10 days, 14 days, 28 days (i.e., one month), or more days prior to the event) that would expose the patient to HIV (or that would otherwise increase the patient's risk of acquiring HIV); and/or (2) during an event (or more than one recurring event) that would expose the patient to HIV (or that would otherwise increase the patient's risk of acquiring HIV); and/or (3) after an event (or after the final event in a series of recurring events) that would expose the patient to HIV (or that would otherwise increase the patient's risk of acquiring HIV). In some embodiments, the event driven administration is performed pre-exposure of the patient to the HIV. In some embodiments, the event driven administration is performed during exposure of the patient to the HIV. In some embodiments, the event driven administration is performed post-exposure of the patient to the HIV.

In some embodiments, the event driven administration is performed pre-exposure of the patient to the HIV and during exposure of the patient to the HIV.

In some embodiments, the event driven administration is performed pre-exposure of the patient to the HIV and post-exposure of the patient to the HIV.

In some embodiments, the event driven administration is performed during exposure of the patient to the HIV and post-exposure of the patient to the HIV.

In certain embodiments, the methods disclosed herein involve administration prior to and/or after an event that would expose the patient to HIV or that would otherwise increase the patient's risk of acquiring HIV, e.g., as pre-exposure prophylaxis (PrEP) and/or as post-exposure prophylaxis (PEP). Examples of events that could increase a patient's risk of acquiring HIV include, without limitation, no condom use during anal intercourse with an HIV positive partner or a partner of unknown HIV status; anal intercourse with more than 3 sexual partners; exchange of money, gifts, shelter or drugs for anal sex; sex with male partner and diagnosis of sexually transmitted infection; and no consistent use of condoms with a sexual partner known to be HIV positive. In some embodiments, the methods disclosed herein comprise pre-exposure prophylaxis (PrEP). In some embodiments, methods disclosed herein comprise post-exposure prophylaxis (PEP). In some embodiments, the methods disclosed herein comprise pre-exposure prophylaxis (PrEP) and post-exposure prophylaxis (PEP).

In some embodiments, a composition provided herein is administered before exposure of the patient to the HIV.

In some embodiments, a composition provided herein is administered during the period of exposure of the patient to the HIV.

In some embodiments, a composition provided herein is administered after final exposure of the patient to the HIV.

In some embodiments, a composition provided herein is administered before and during exposure of the patient to the HIV.

In some embodiments, a composition provided herein is administered before and after exposure of the patient to the HIV.

In some embodiments, a composition provided herein is administered during and after exposure of the patient to the HIV.

In some embodiments, a composition provided herein is administered before, during, and after exposure of the patient to the HIV.

In some embodiments, the dose of the composition administered during each period (i.e., before, during, and after exposure) may be different, i.e, independently selected from any of the doses disclosed herein.

In certain embodiments, e.g., when administered as PrEP, a composition provided herein is administered 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) prior to an event that would increase the patient's risk of acquiring HIV (e.g., prior to sexual intercourse or other exposure to the HIV). In some embodiments, a composition provided herein is administered within 14 days, 13 days, 12 days, 11 days, 10 days, 9 days, 8 days, 7 days, 6 days, 5 days, 4 days, 3 days, 2 days, or 1 day prior to an event that would increase the patient's risk of acquiring HIV (e.g., prior to sexual intercourse or other exposure to the HIV). In some embodiments, a composition provided herein is administered within 72 hours, 60 hours, 48 hours, 24 hours, 12 hours, 9 hours, 6 hours, 4 hours, 3 hours, 2 hours, or 1 hour prior to an event that would increase the patient's risk of acquiring HIV (e.g., prior to sexual intercourse or other exposure to the HIV). In certain embodiments, when a composition provided herein is administered prior to an event that would increase the patient's risk of acquiring HIV, it is administered daily prior to the event (e.g., sexual activity). In certain embodiments, when a composition provided herein is administered prior to an event that would increase the patient's risk of acquiring HIV, it is administered one to three times prior to the event. In certain embodiments, when a composition provided herein is administered prior to an event that would increase the patient's risk of acquiring HIV, it is administered one time (i.e., once) prior to the event.

In some embodiments, a composition provided herein is administered from about 14 days to about one day before exposure of the patient to the HIV. In some embodiments, a composition provided herein is administered once from about 14 days to about one day before exposure of the patient to the HIV.

In some embodiments, a composition provided herein is administered from about 10 days to about 5 days before exposure of the patient to the HIV. In some embodiments, a composition provided herein is administered once from about 10 days to about 5 days before exposure of the patient to the HIV.

In some embodiments, a composition provided herein is administered from about 8 days to about 6 days before exposure of the patient to the HIV. In some embodiments, a composition provided herein is administered once from about 8 days to about 6 days before exposure of the patient to the HIV.

In some embodiments, a composition provided herein is administered about 7 days before exposure of the patient to the HIV. In some embodiments, a composition provided herein is administered once about 7 days before exposure of the patient to the HIV.

In some embodiments, a composition provided herein is administered from about 72 hours to about 1 hour before exposure of the patient to the HIV. In some embodiments, a composition provided herein is administered once from about 72 hours to about 1 hour before exposure of the patient to the HIV.

In some embodiments of the methods provided herein, the pre-exposure prophylaxis (PrEP) comprises continuous PrEP.

In certain embodiments where a composition provided herein is administered before exposure of the patient to the HIV, the methods disclosed herein further comprise administering one or more additional doses of the compound of Formula Ia or Ib, or a pharmaceutically acceptable salt or free acid form thereof, during, and/or after exposure of the patient to the HIV.

In some embodiments, e.g., when administered as part of a PrEP regimen or as part of a PEP regimen, a composition provided herein is administered during the period of exposure of the patient to the HIV. In certain embodiments wherein a composition provided herein is administered before HIV exposure, the composition is administered about every 7 days, about every 14 days, about every 21 days, about every 28 days, about every 35 days, about every 42 days, or about every 6 months, or about every 12 months (e.g., as a single dose) during the time of HIV exposure (e.g., during the time period of sexual activity with a sexual partner known to be HIV positive). In some embodiments, a composition provided herein is administered once about every 7 days, about every 14 days, about every 21 days, about every 28 days, about every 35 days, about every 42 days, about every 6 months, or about every 12 months during the period of exposure of the patient to the HIV.

In some embodiments, a composition provided herein administered prior to exposure to the HIV is at a different dose than a composition (e.g., a composition comprising a compound of Formula Ia or Ib, or a pharmaceutically acceptable salt or free acid form thereof) administered during and/or after exposure to the HIV. For example, in some embodiments, the dose of the compound of Formula Ia or Ib, or a pharmaceutically acceptable salt or free acid form thereof, is increased, e.g., as a double dose, as a triple dose, and the like as compared to an earlier administered dose (e.g., a dose prior to exposure to the HIV). In some embodiments, the increased dose of the compound of Formula Ia or Ib, or a pharmaceutically acceptable salt or free acid form thereof, is a double dose. In some embodiments, the dose of the compound of Formula Ia or Ib, or a pharmaceutically acceptable salt or free acid form thereof, is decreased, e.g., a half dose as compared to an earlier administered dose (e.g., a dose prior to exposure to the HIV).

In some embodiments, a composition provided herein (e.g., a composition comprising a compound of Formula Ia or Ib, or a pharmaceutically acceptable salt or free acid form thereof) is administered as a single dose from about 1 hour to about 10 days before exposure of the patient to the HIV.

Additional examples of PrEP and/or PEP can be found, for example, at the clinical trial summary titled “On Demand Antiretroviral Pre-exposure Prophylaxis for HIV Infection in Men Who Have Sex With Men” (Clinical Trial #NCT01473472); the clinical trial summary titled “Prevention of HIV in Île-de-France” (Clinical Trials #NCT03113123), and at Molina et al, N. Engl. J. Med. 2015, 353:2237-2246, the disclosure of each of which is incorporated herein by reference in its entirety.

In some embodiments, e.g., when administered as part of a PrEP regimen or as part of a PEP regimen, a composition provided herein is administered 1 hour to 10 days, 1 hour to 7 days, 1 hour to 5 days, 1 to 72 hours, 1 to 48 hours, 1 to 36 hours, 1 to 24 hours, or 1 to 12 hours following an event that would increase the patient's risk of acquiring HIV (e.g., following sexual intercourse or other exposure to the HIV).

In certain embodiments, e.g., when administered as PEP, a composition provided herein is administered for 7 days, 14 days, 21 days, 28 days, 30 days, or 45 days following an event that would increase the patient's risk of acquiring HIV (e.g., following sexual intercourse or other exposure to the HIV). In certain embodiments, e.g., when administered as PEP, a composition provided herein is administered for 30 days following an event that would increase the patient's risk of acquiring HIV (e.g., following sexual intercourse or other exposure to the HIV). In certain embodiments, a composition provided herein is administered less than 1 hour, 2 hours, 3 hours, 4 hours, 5 hours, 6 hours, 7 hours, 8 hours, 9 hours, 12 hours, 18 hours, 24 hours, 36 hours, or 48 hours following an event that would increase the patient's risk of acquiring HIV (e.g., following sexual intercourse or other exposure to the HIV virus). In certain embodiments, a composition provided herein is administered for 1 day, 2 days, 3 days, 4 days, or 5 days following an event that would increase the patient's risk of acquiring HIV (e.g., following sexual intercourse or other exposure to the HIV). In certain embodiments, when a composition provided herein is administered following an event that would increase the patient's risk of acquiring HIV, it is administered daily following the event. In certain embodiments, when a composition provided herein is administered following an event that would increase the patient's risk of acquiring HIV, it is administered one to three times following the event. In certain embodiments, when a composition provided herein is administered following an event that would increase the patient's risk of acquiring HIV, it is administered once following the event.

In certain embodiments, e.g., when administered as PEP, a composition provided herein is administered once about every week, once about every month, once about every 2 months, once about every 3 months, once about every 4 months, once about every 5 months, once about every 6 months, or once about every 12 months following an event that would increase the patient's risk of acquiring HIV (e.g., following sexual intercourse or other exposure to the HIV). In certain embodiments, e.g., when administered as PEP, a composition provided herein is administered once about every week following an event that would increase the patient's risk of acquiring HIV (e.g., following sexual intercourse or other exposure to the HIV). In certain embodiments, e.g., when administered as PEP, a composition provided herein is administered once about every month following an event that would increase the patient's risk of acquiring HIV (e.g., following sexual intercourse or other exposure to the HIV). In certain embodiments, e.g., when administered as PEP, a composition provided herein is administered once about every 2 months following an event that would increase the patient's risk of acquiring HIV (e.g., following sexual intercourse or other exposure to the HIV). In certain embodiments, e.g., when administered as PEP, a composition provided herein is administered once about every 3 months following an event that would increase the patient's risk of acquiring HIV (e.g., following sexual intercourse or other exposure to the HIV). In certain embodiments, e.g., when administered as PEP, a composition provided herein is administered once about every 4 months following an event that would increase the patient's risk of acquiring HIV (e.g., following sexual intercourse or other exposure to the HIV). In certain embodiments, e.g., when administered as PEP, a composition provided herein is administered once about every 5 months following an event that would increase the patient's risk of acquiring HIV (e.g., following sexual intercourse or other exposure to the HIV). In certain embodiments, e.g., when administered as PEP, a composition provided herein is administered once about every 6 months following an event that would increase the patient's risk of acquiring HIV (e.g., following sexual intercourse or other exposure to the HIV). In certain embodiments, e.g., when administered as PEP, a composition provided herein is administered once about every 12 months following an event that would increase the patient's risk of acquiring HIV (e.g., following sexual intercourse or other exposure to the HIV).

In certain embodiments, e.g., when administered as PEP, a composition provided herein is administered for one month, two months, three months, four months, five months, six months, or twelve months following an event that would increase the patient's risk of acquiring HIV (e.g., following sexual intercourse or other exposure to the HIV).

In certain embodiments, when a composition provided herein is administered following an event that would increase the patient's risk of acquiring HIV, it is administered one to fifty times following the event. In certain embodiments, when a composition provided herein is administered following an event that would increase the patient's risk of acquiring HIV, it is administered one to forty times following the event. In certain embodiments, when a composition provided herein is administered following an event that would increase the patient's risk of acquiring HIV, it is administered one to thirty times following the event. In certain embodiments, when a composition provided herein is administered following an event that would increase the patient's risk of acquiring HIV, it is administered one to twenty times following the event. In certain embodiments, when a composition provided herein is administered following an event that would increase the patient's risk of acquiring HIV, it is administered one to fifteen times following the event. In certain embodiments, when a composition provided herein is administered following an event that would increase the patient's risk of acquiring HIV, it is administered one to ten times following the event. In certain embodiments, when a composition provided herein is administered following an event that would increase the patient's risk of acquiring HIV, it is administered one to five times following the event.

In some embodiments, a composition provided herein is administered during exposure of the patient to the HIV (e.g., during a period of sexual activity with a sexual partner known to be HIV positive).

In some embodiments, a composition provided herein is administered after exposure (e.g., after final exposure) of the patient to the HIV (e.g., after a period of sexual activity with a sexual partner known to be HIV positive). In some embodiments, a composition provided herein is administered from about 1 hour to about 14 days after exposure (e.g., after final exposure) of the patient to the HIV. In some embodiments, a composition provided herein is administered once from about 1 hour to about 14 days after exposure (e.g., after final exposure) of the patient to the HIV. In some embodiments, a composition provided herein is administered from about 1 hour to about 7 days after exposure (e.g., after final exposure) of the patient to the HIV. In some embodiments, a composition provided herein is administered once from about 1 hour to about 7 days after exposure (e.g., after final exposure) of the patient to the HIV. In some embodiments, a composition provided herein is administered from about 1 hour to about 72 hours after exposure (e.g., after final exposure) of the patient to the HIV. In some embodiments, a composition provided herein is administered once from about 1 hour to about 72 hours after exposure (e.g., after final exposure) of the patient to the HIV. In some embodiments, a composition provided herein is administered from about 1 hour to about 24 hours after exposure (e.g., after final exposure) of the patient to the HIV. In some embodiments, a composition provided herein is administered once from about 1 hour to about 24 hours after exposure (e.g., after final exposure) of the patient to the HIV. In some embodiments, a composition provided herein is administered from about 24 hours to about 72 hours after exposure (e.g., after final exposure) of the patient to the HIV. In some embodiments, a composition provided herein is administered once from about 24 hours to about 72 hours after exposure (e.g., after final exposure) of the patient to the HIV.

In some embodiments, e.g., when administered as PrEP, a composition provided herein is administered prior to an event that would increase the patient's risk of acquiring HIV (e.g., prior to sexual activity), and following the event. For example, in certain embodiments, when administered as PrEP, a composition provided herein is administered 1 to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) and 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 36 hours, 1 hour to 24 hours, or 1 hour to 12 hours following the event. For example, in some embodiments, one or more (e.g., one, two, or three) dosages of a composition provided herein are administered one to ten days (e.g., seven days) prior to an event that would increase the patient's risk of acquiring HIV (e.g., prior to sexual intercourse) and once during a period of one to ten days following the event. In some embodiments, a composition provided herein is administered once per week, twice per week, three times per week, four times per week, or five times per week and one or more times (e.g., one, two, or three times) beginning 1 to 48 hours following an event that would increase the patient's risk of acquiring HIV (e.g., following sexual intercourse).

In some embodiments, a composition provided herein is administered to a patient once every one month to once every fourteen months, for example, once every month, once every two months, once every three months, once every four months, once every five months, once every six months, once every seven months, once every eight months, once every nine months, once every ten months, once every eleven months, once every twelve months, once every thirteen months, or once every fourteen months. In some embodiments, a composition provided herein is administered to a patient once every four months. In some embodiments, a composition provided herein is administered to a patient once every six months. In some embodiments, a composition provided herein is administered to a patient once every twelve months.

Also provided herein is a method of reducing the risk of acquiring HIV in a patient, comprising administering to the patient a composition provided herein (e.g., a composition comprising a compound of Formula Ia or Ib, or a pharmaceutically acceptable salt or free acid form thereof).

In some embodiments, the present disclosure relates to a method of reducing the risk of sexually acquired HIV in a patient comprising administering to the patient a composition provided herein (e.g., a composition comprising a compound of Formula Ia or Ib, or a pharmaceutically acceptable salt or free acid form thereof).

In some embodiments, the present disclosure relates to a method of reducing the risk of sexually acquired HIV in a patient, comprising administering to the patient a compound of

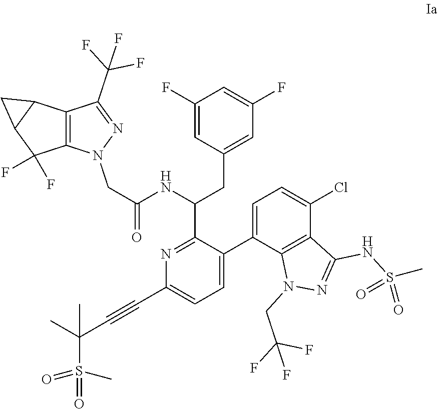 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every twelve months from the first day, wherein the composition comprises:
   about 30% to about 45% of the free acid form of the compound of Formula Ia;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

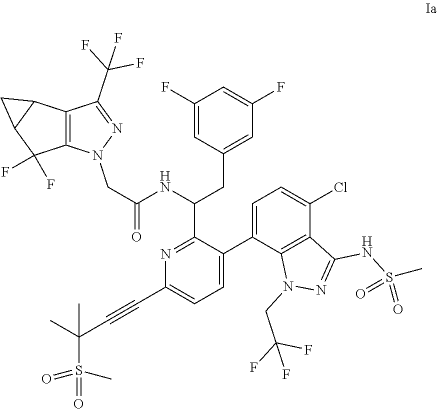 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1200-1800 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 30% to about 45% of the free acid form of the compound of Formula Ia;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

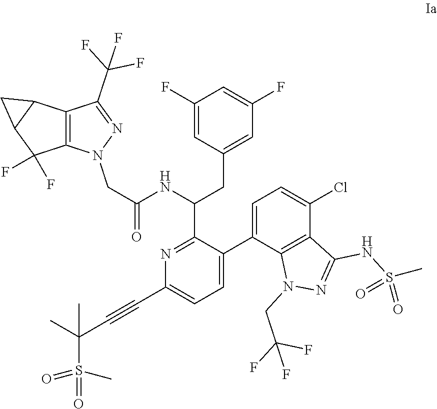 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1400-1600 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 30% to about 45% of the free acid form of the compound of Formula Ia;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

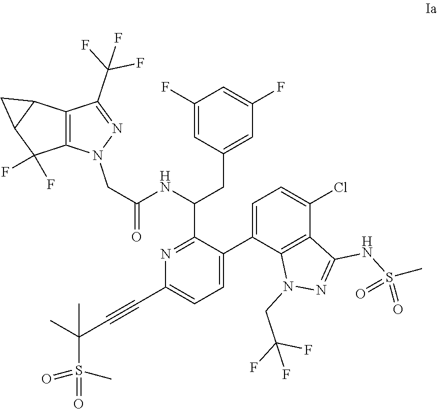 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 30% to about 45% of the free acid form of the compound of Formula Ia;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

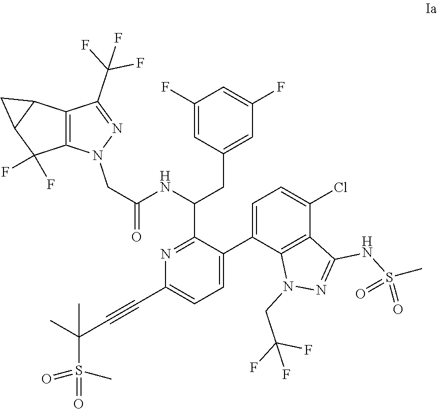 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1200-1800 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

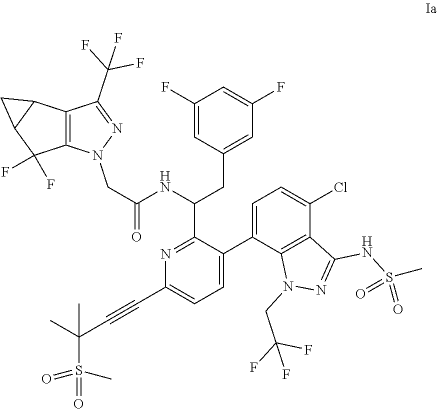 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1200-1800 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

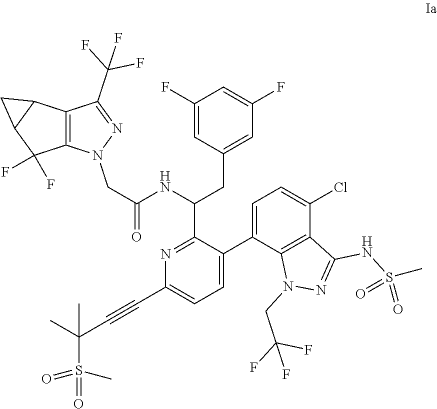 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1200-1800 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

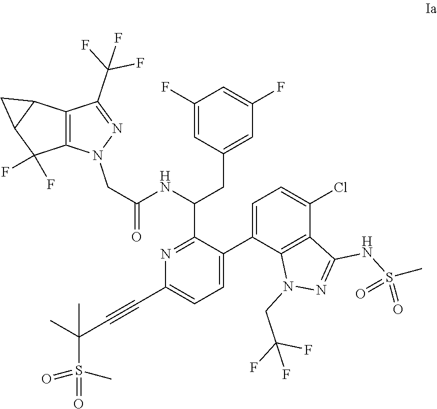 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1400-1600 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

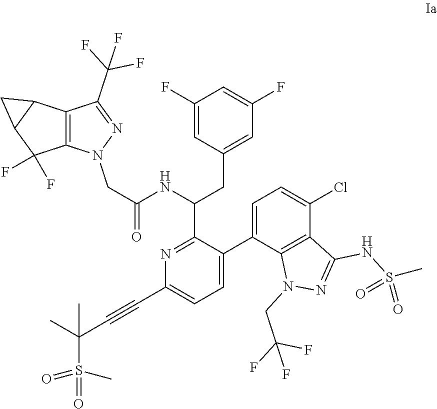 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1400-1600 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

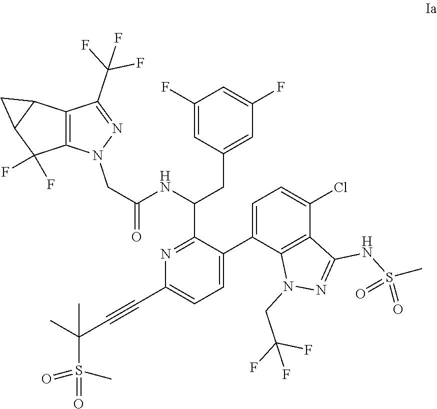 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1400-1600 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

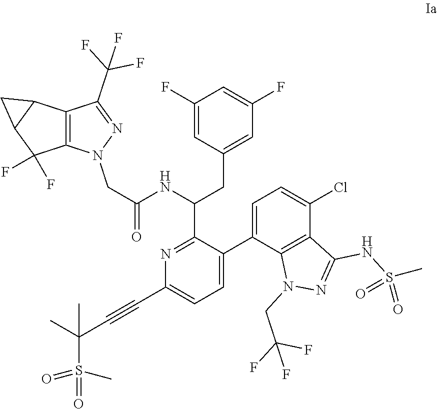 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 800-1200 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 30% to about 45% of the free acid form of the compound of Formula Ia;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

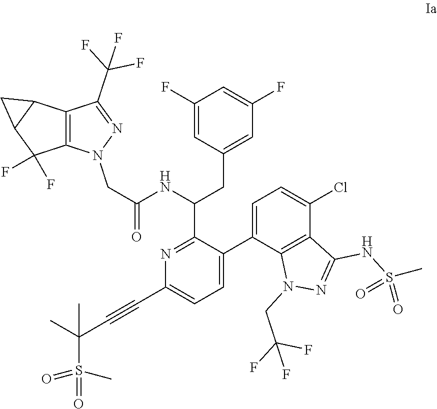 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 900-1100 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 30% to about 45% of the free acid form of the compound of Formula Ia;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

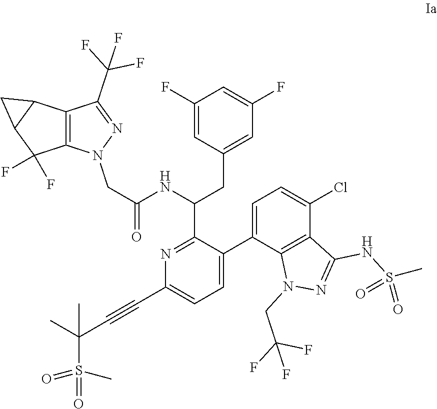 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 30% to about 45% of the free acid form of the compound of Formula Ia;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

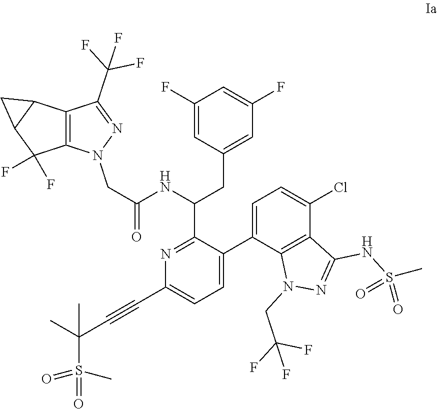 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 800-1200 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

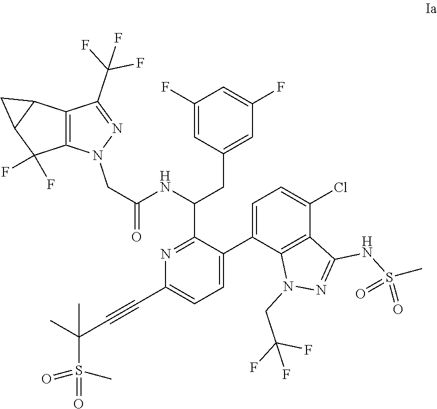 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 800-1200 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

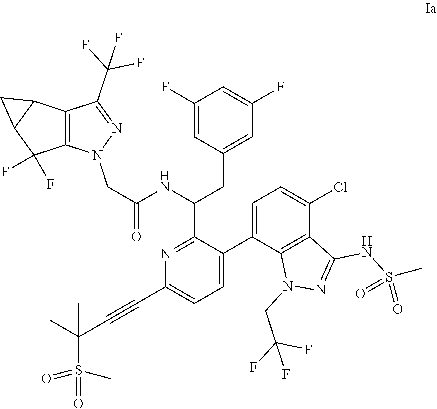 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 800-1200 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

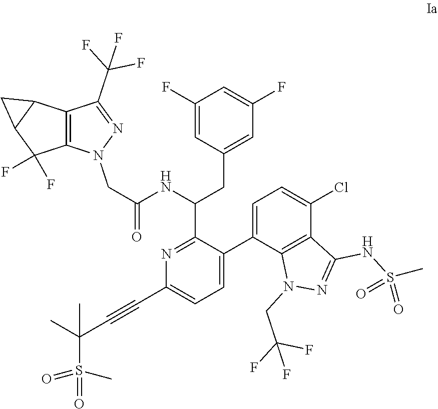 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 900-1100 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

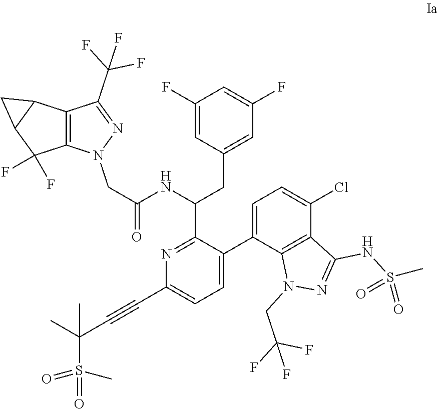 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 900-1100 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

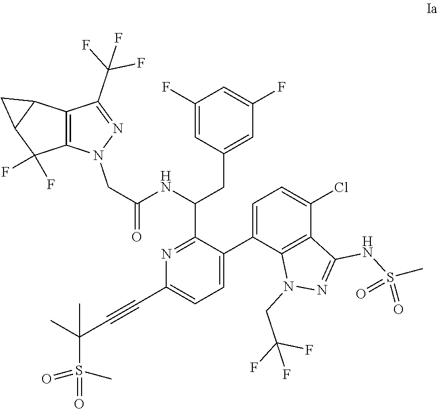 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 900-1100 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

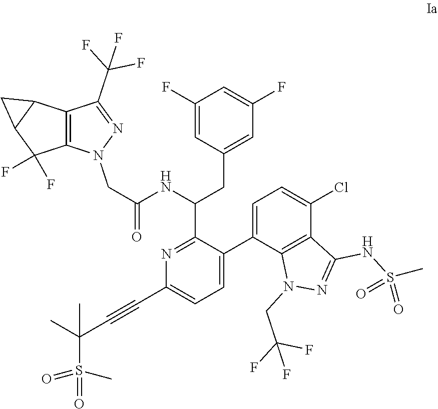 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every twelve months from the first day, wherein the composition comprises:
   about 30% to about 45% of the free acid form of the compound of Formula Ia;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

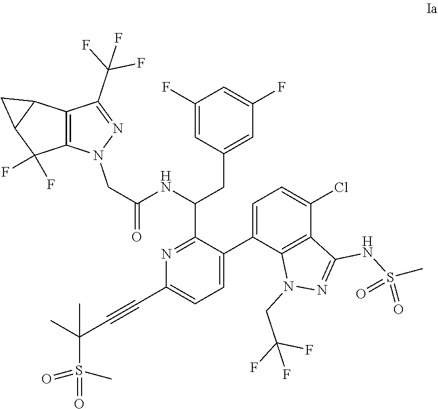 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every twelve months from the first day, wherein the composition comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

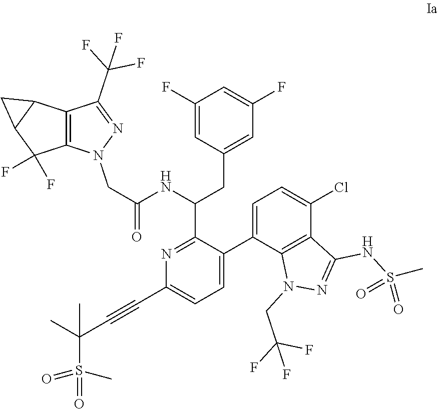 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every twelve months from the first day, wherein the composition comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

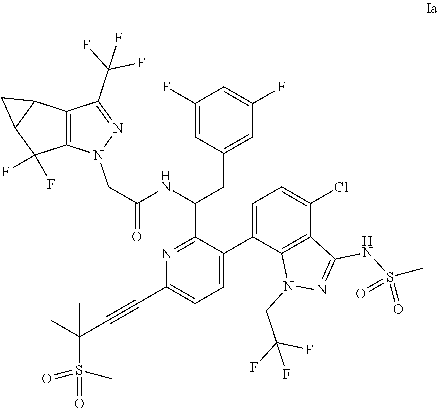 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every twelve months from the first day, wherein the composition comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

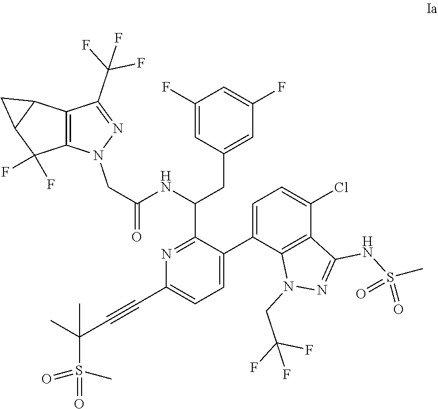 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 3000 mg of the free acid form of the compound of Formula Ia, as two intramuscular injections of about 3 mL each, each injection comprising about 1500 mg of the free acid form of the compound of Formula Ia, once every twelve months from the first day, wherein each intramuscular injection comprises:
   about 30% to about 45% of the free acid form of the compound of Formula Ia;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

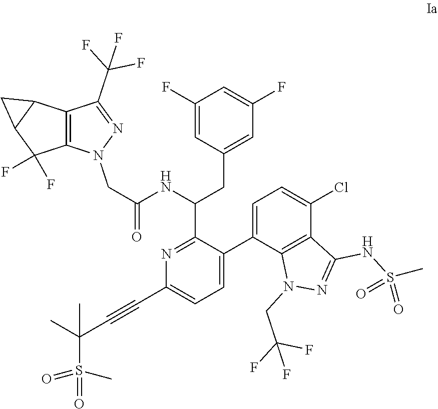 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 3000 mg of the free acid form of the compound of Formula Ia, as two intramuscular injections of about 3 mL each, each injection comprising about 1500 mg of the free acid form of the compound of Formula Ia, once every twelve months from the first day, wherein each intramuscular injection comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

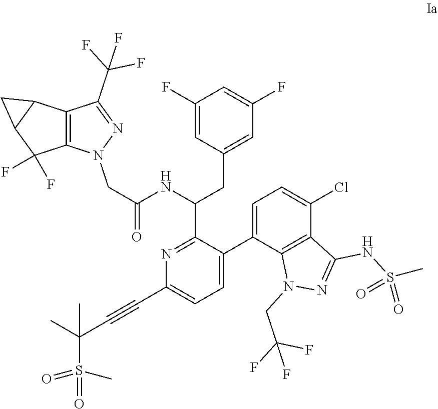 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 3000 mg of the free acid form of the compound of Formula Ia, as two intramuscular injections of about 3 mL each, each injection comprising about 1500 mg of the free acid form of the compound of Formula Ia, once every twelve months from the first day, wherein each intramuscular injection comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

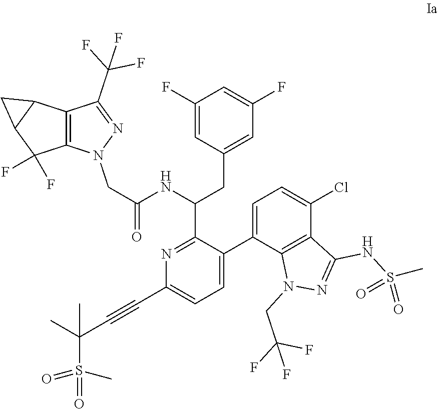 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 3000 mg of the free acid form of the compound of Formula Ia, as two intramuscular injections of about 3 mL each, each injection comprising about 1500 mg of the free acid form of the compound of Formula Ia, once every twelve months from the first day, wherein each intramuscular injection comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

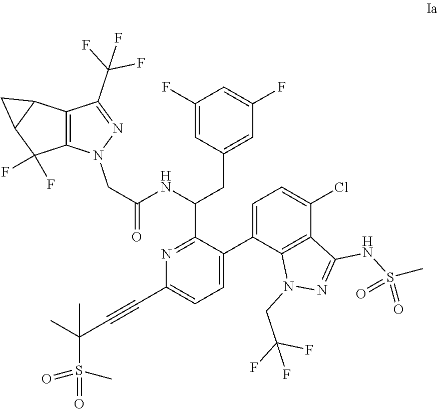 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 30% to about 45% of the free acid form of the compound of Formula Ia;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

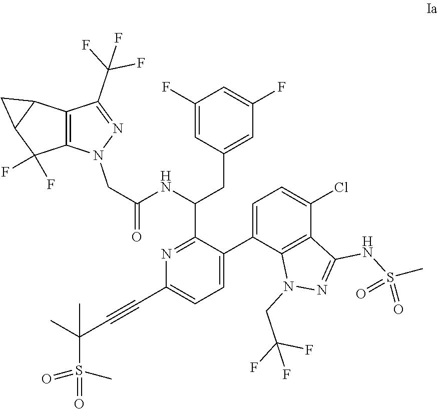 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

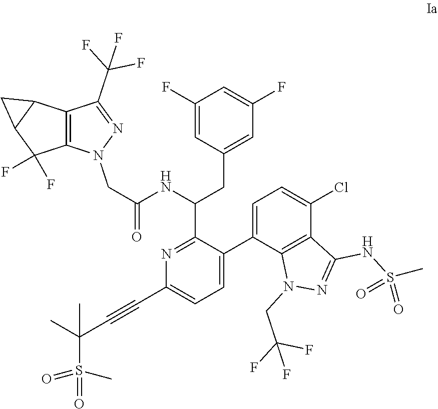 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

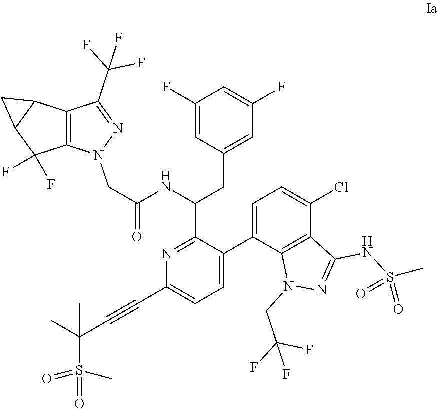 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every six months from the first day, wherein the composition comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

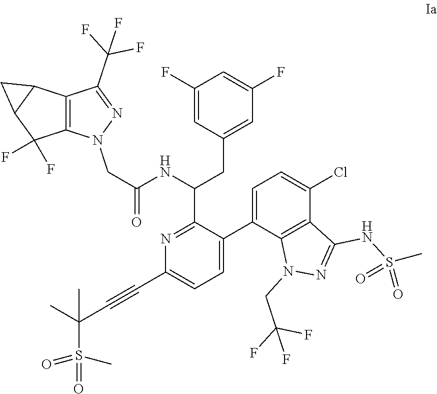 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1500 mg of the free acid form of the compound of Formula Ia, as one intramuscular injection of about 3 mL, every six months from the first day, wherein the intramuscular injection comprises:
   about 30% to about 45% of the free acid form of the compound of Formula Ia;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

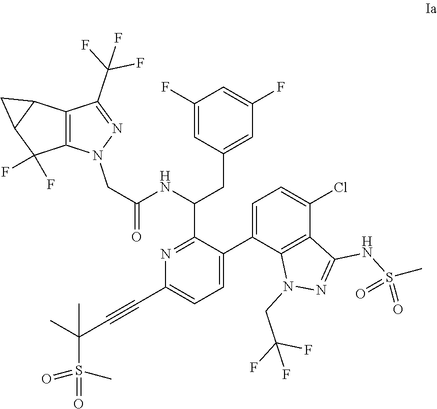 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1500 mg of the free acid form of the compound of Formula Ia, as one intramuscular injection of about 3 mL, every six months from the first day, wherein the intramuscular injection comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

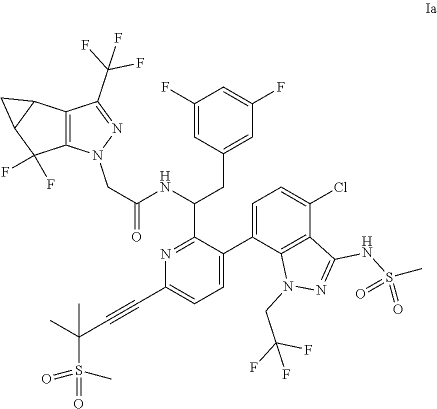 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1500 mg of the free acid form of the compound of Formula Ia, as one intramuscular injection of about 3 mL, every six months from the first day, wherein the intramuscular injection comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

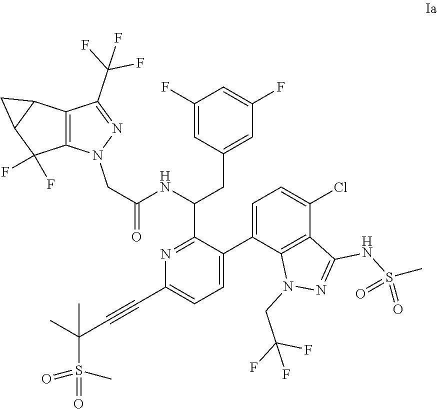 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1500 mg of the free acid form of the compound of Formula Ia, as one intramuscular injection of about 3 mL, every six months from the first day, wherein the intramuscular injection comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

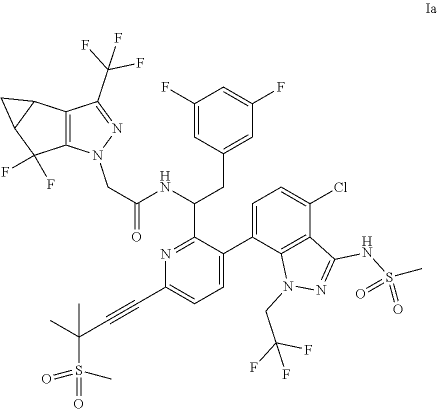 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 30% to about 45% of the free acid form of the compound of Formula Ia;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

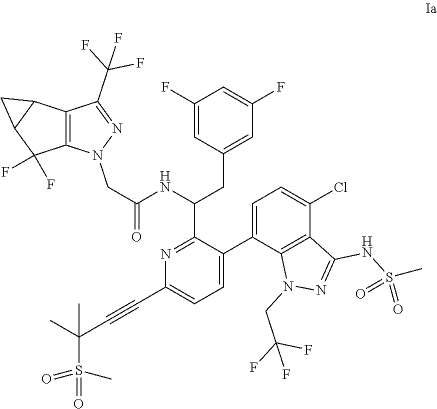 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

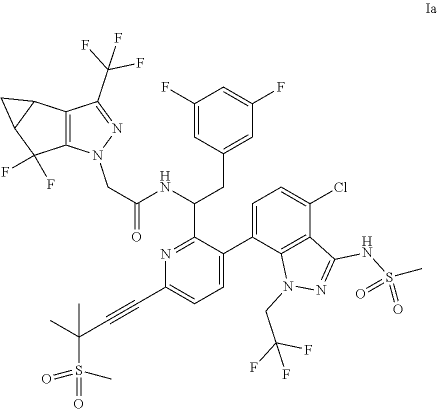 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

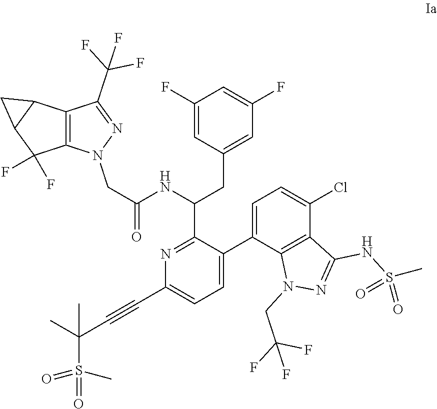 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every four months from the first day, wherein the composition comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

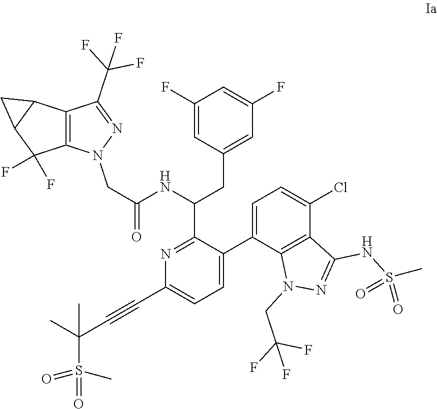 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1000 mg of the free acid form of the compound of Formula Ia, as one intramuscular injection of about 2 mL, every four months from the first day, wherein the intramuscular injection comprises:
   about 30% to about 45% of the free acid form of the compound of Formula Ia;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

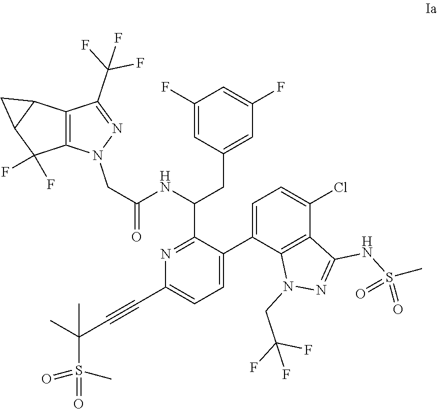 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1000 mg of the free acid form of the compound of Formula Ia, as one intramuscular injection of about 2 mL, every four months from the first day, wherein the intramuscular injection comprises:
   about 41% to about 43% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

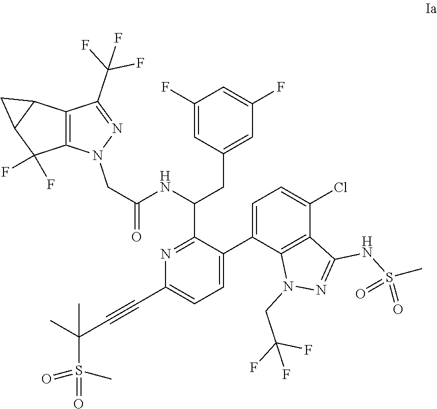 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1000 mg of the free acid form of the compound of Formula Ia, as one intramuscular injection of about 2 mL, every four months from the first day, wherein the intramuscular injection comprises:
   about 42% of the free acid form of the compound of Formula Ia;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

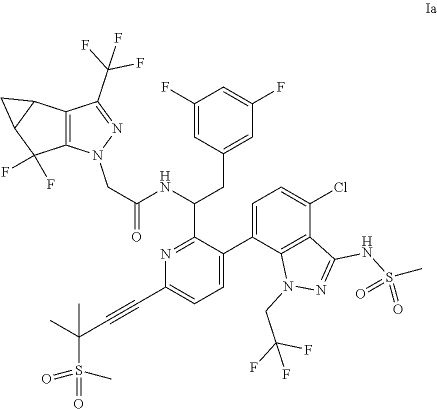 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ia, or a pharmaceutically acceptable salt thereof, on the first day;
   orally administering to the patient about 600 mg of the compound of Formula Ia, or a pharmaceutically acceptable salt thereof, on the first day; and
   orally administering to the patient about 600 mg of the compound of Formula Ia, or a pharmaceutically acceptable salt thereof, on the second day; and
(ii) intramuscularly administering to the patient about 1000 mg of the free acid form of the compound of Formula Ia, as one intramuscular injection of about 2 mL, every four months from the first day, wherein the intramuscular injection comprises:
   about 42.02% of the free acid form of the compound of Formula Ia;
   about 37% to about 43% of the PEG 300;
   about 9% to about 11% of the ethanol; and
   about 6% to about 8% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 30% to about 45% of the free acid form of the compound of Formula Ia;
about 40% to about 50% of the PEG 300;
about 7% to about 15% of the ethanol; and
about 5% to about 10% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 40% to about 43% of the free acid form of the compound of Formula Ia;
about 40% to about 42% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 40% to about 43% of the free acid form of the compound of Formula Ia;
about 40% to about 42% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 42% to about 43% of the free acid form of the compound of Formula Ia;
about 40% to about 41% of the PEG 300;
about 9% to about 11% of the ethanol; and
about 7% to about 8% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 41% to about 43% of the free acid form of the compound of Formula Ia;
about 37% to about 43% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 42% of the free acid form of the compound of Formula Ia;
about 37% to about 43% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 42.02% of the free acid form of the compound of Formula Ia;
about 37% to about 43% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ia, PEG 300, ethanol, and water) comprises:
about 42.02% of the free acid form of the compound of Formula Ia;
about 40.78% of the PEG 300;
about 10% of the ethanol; and
about 7.20% of the water.

In some embodiments of the previous embodiments, the intramuscular injection of step (ii) (i.e., the pharmaceutical composition comprising a free acid form of the compound of Formula Ia, PEG 300, ethanol, and water) comprises administering to the patient two intramuscular injections of about 3 mL each, once every twelve months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 3000 mg of the free acid form of the compound of Formula Ia, once every twelve months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 3000 mg of the free acid form of the compound of Formula Ia, as two intramuscular injections of about 1500 mg, once every twelve months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 3000 mg of the free acid form of the compound of Formula Ia, as two 3 mL intramuscular injections of about 1500 mg each, once every twelve months from the first day.

In some embodiments of the previous embodiments, the intramuscular injection of step (ii) (i.e., the pharmaceutical composition comprising a free acid form of the compound of Formula Ia, PEG 300, ethanol, and water) comprises administering to the patient one intramuscular injection of about 3 mL, once every six months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 1500 mg of the free acid form of the compound of Formula Ia, once every six months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 1500 mg of the free acid form of the compound of Formula Ia, as one intramuscular injection, once every six months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 1500 mg of the free acid form of the compound of Formula Ia, as one 3 mL intramuscular injection, once every six months from the first day.

In some embodiments of the previous embodiments, the intramuscular injection of step (ii) (i.e., the pharmaceutical composition comprising a free acid form of the compound of Formula Ia, PEG 300, ethanol, and water) comprises administering to the patient one intramuscular injection of about 2 mL, once every four months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 1000 mg of the free acid form of the compound of Formula Ia, once every four months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 1000 mg of the free acid form of the compound of Formula Ia, as one intramuscular injection, once every four months from the first day.

In some embodiments of the previous embodiments, the intramuscular injection of step (ii) (i.e., the pharmaceutical composition comprising a free acid form of the compound of Formula Ia, PEG 300, ethanol, and water) comprises intramuscularly administering to the patient about 1000 mg of the free acid form of the compound of Formula Ia, as one 2 mL intramuscular injection, once every four months from the first day.

In some embodiments, the present disclosure relates to a method of reducing the risk of sexually acquired HIV in a patient, comprising administering to the patient a compound of

### Formula Ib:

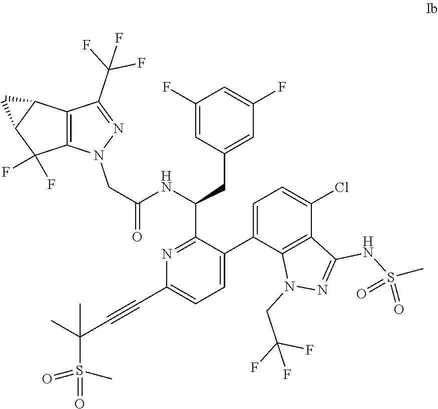 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every twelve months from the first day, wherein the composition comprises:
   about 30% to about 45% of the free acid form of the compound of Formula Ib;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of Formula Ib:

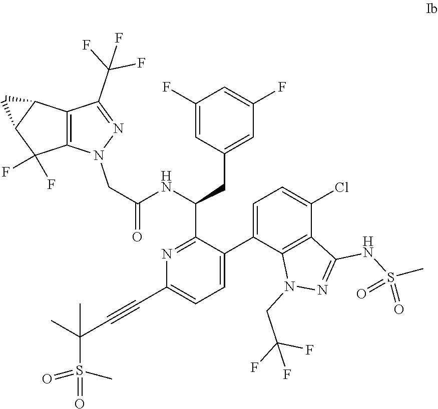 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1200 to about 1800 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 30% to about 45% of the free acid form of the compound of Formula Ib;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

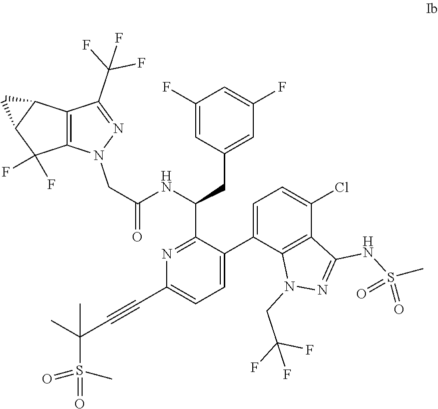 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1400 to about 1600 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 30% to about 45% of the free acid form of the compound of Formula Ib;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

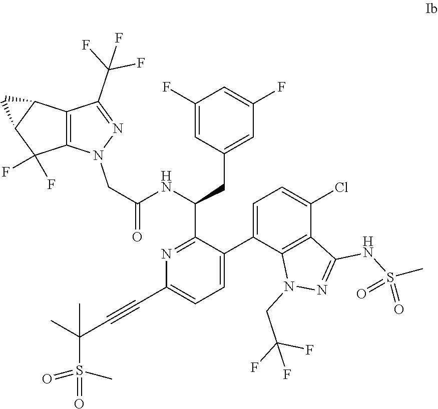 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 30% to about 45% of the free acid form of the compound of Formula Ib;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

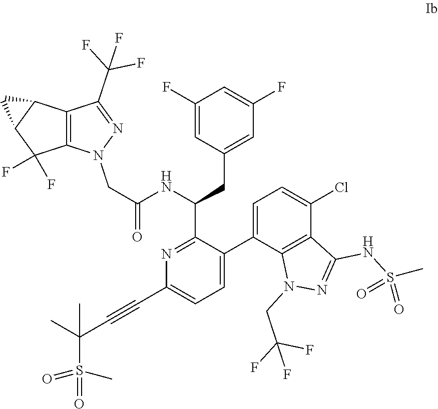 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1200 to about 1800 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

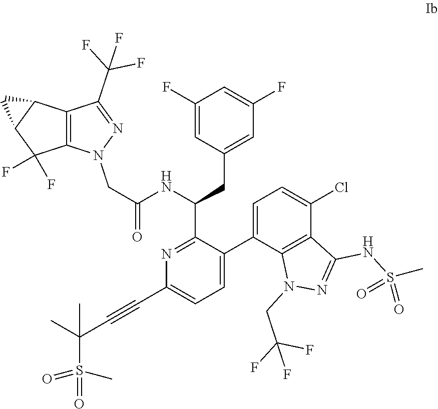 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1200 to about 1800 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

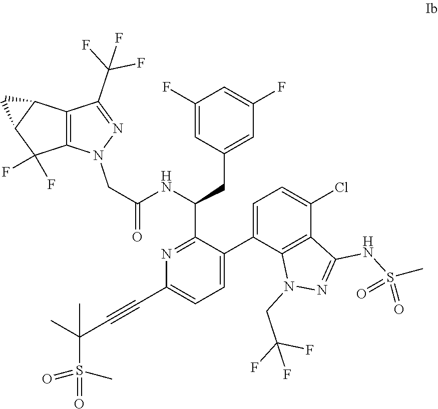 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1200 to about 1800 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

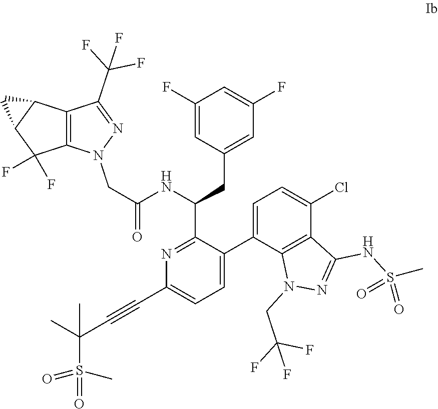 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1400 to about 1600 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

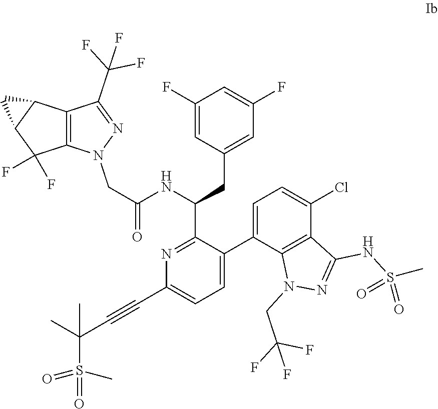 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1400 to about 1600 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

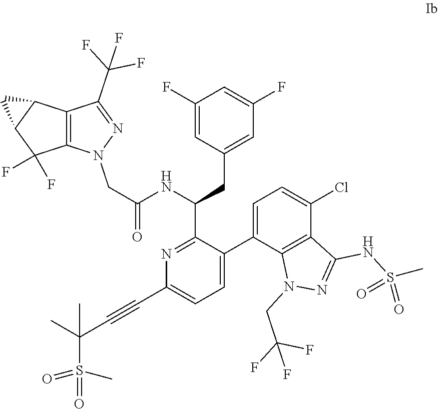 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1400 to about 1600 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

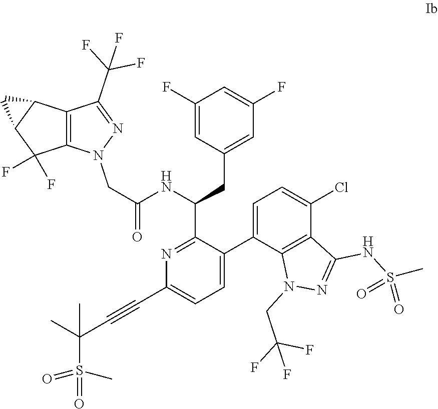 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 800 to about 1200 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 30% to about 45% of the free acid form of the compound of Formula Ib;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

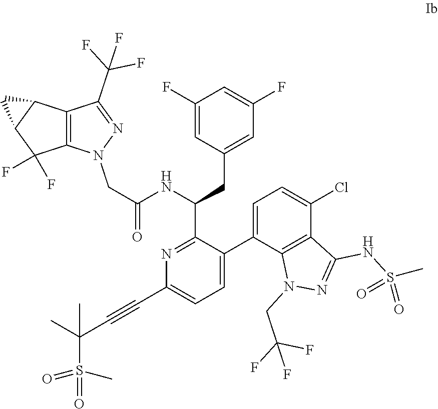 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 900 to about 1100 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 30% to about 45% of the free acid form of the compound of Formula Ib;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

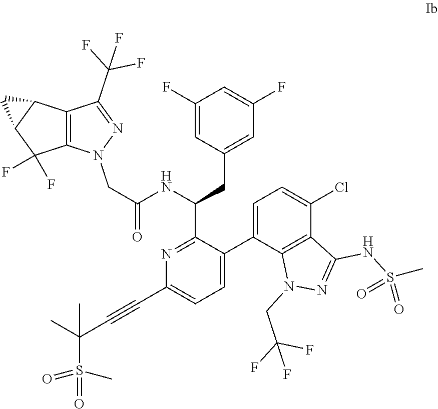 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 30% to about 45% of the free acid form of the compound of Formula Ib;
   about 37% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

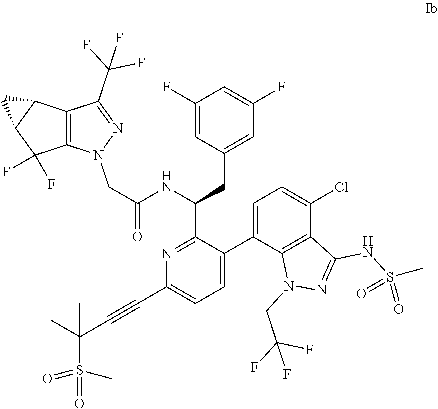 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 800 to about 1200 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

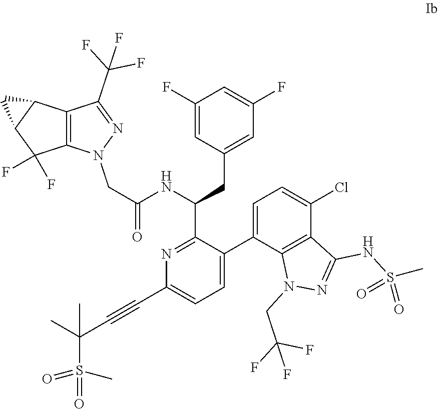 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 800 to about 1200 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

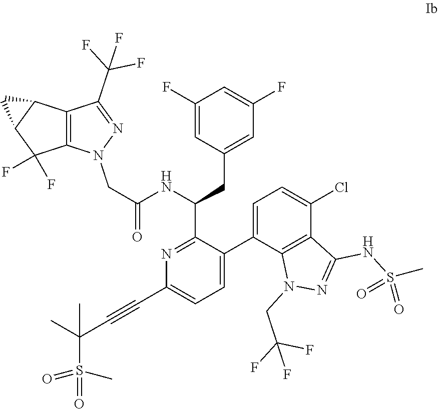 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 800 to about 1200 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

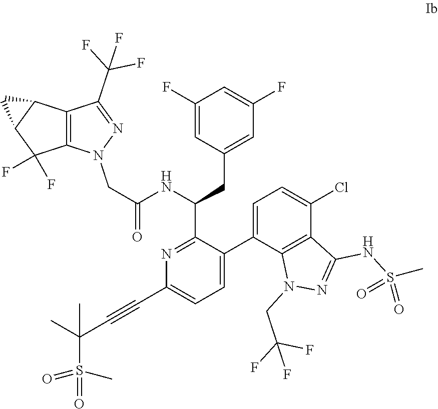 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 900 to about 1100 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

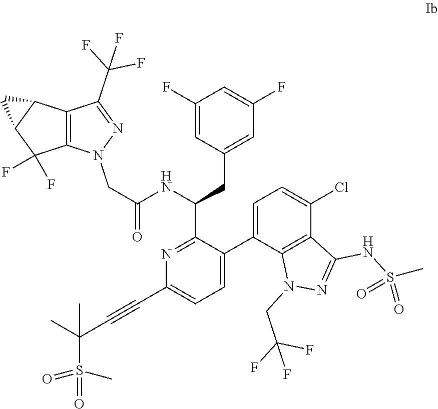 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 900 to about 1100 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

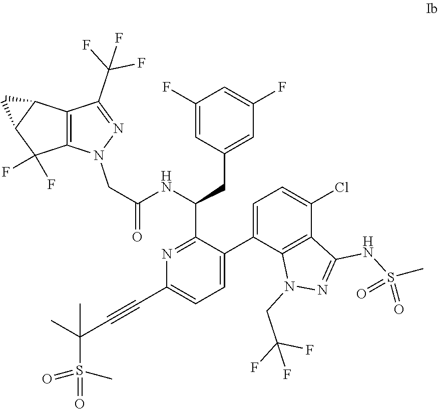 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 900 to about 1100 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

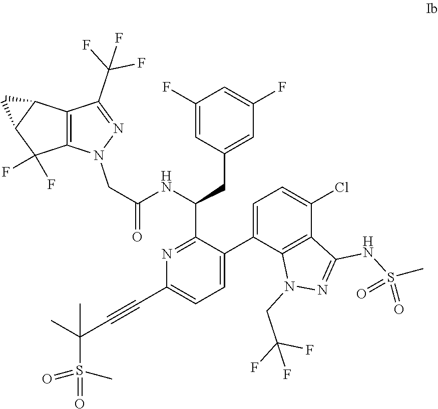 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every twelve months from the first day, wherein the composition comprises:
   about 30% to about 45% of the free acid form of the compound of Formula Ib;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

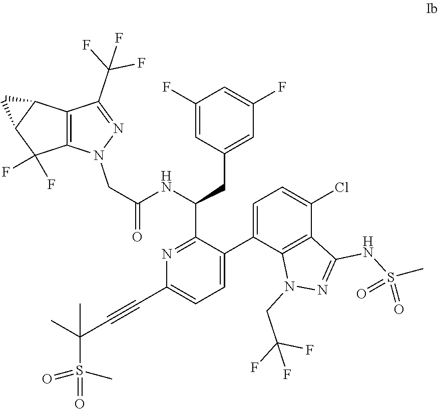 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every twelve months from the first day, wherein the composition comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

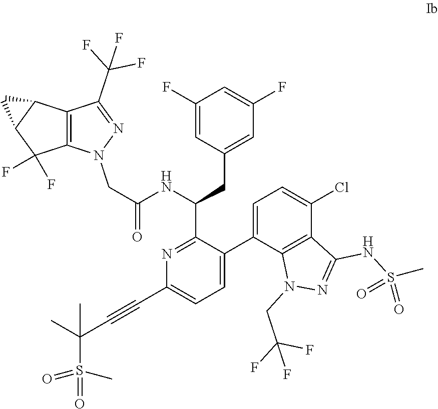 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every twelve months from the first day, wherein the composition comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

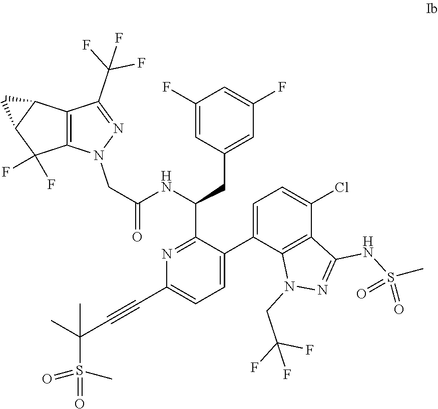 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every twelve months from the first day, wherein the composition comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

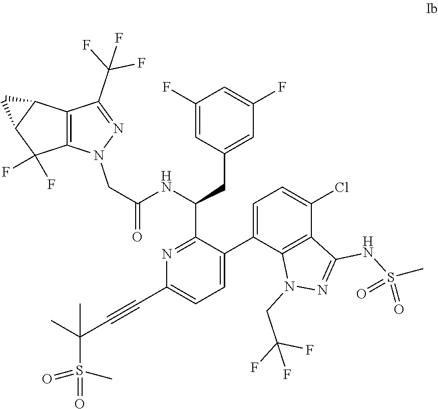 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 3000 mg of the free acid form of the compound of Formula Ib, as two intramuscular injections of about 3 mL each, each injection comprising about 1500 mg of the free acid form of the compound of Formula Ib, once every twelve months from the first day, wherein each intramuscular injection comprises:
   about 30% to about 45% of the free acid form of the compound of Formula Ib;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

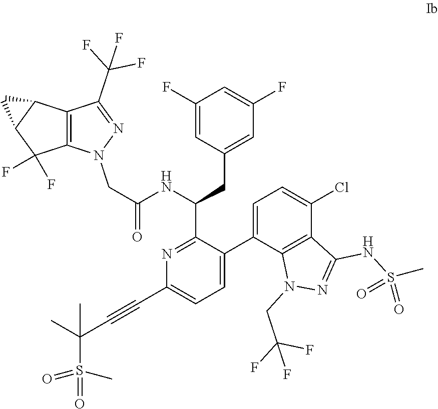 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 3000 mg of the free acid form of the compound of Formula Ib, as two intramuscular injections of about 3 mL each, each injection comprising about 1500 mg of the free acid form of the compound of Formula Ib, once every twelve months from the first day, wherein each intramuscular injection comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

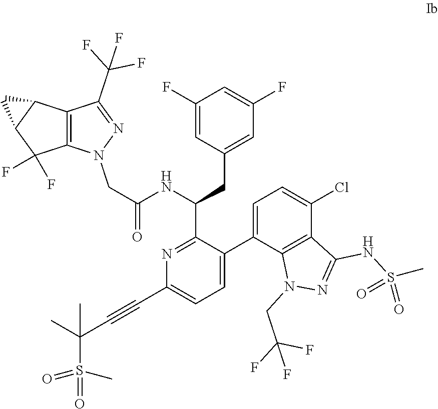 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 3000 mg of the free acid form of the compound of Formula Ib, as two intramuscular injections of about 3 mL each, each injection comprising about 1500 mg of the free acid form of the compound of Formula Ib, once every twelve months from the first day, wherein each intramuscular injection comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

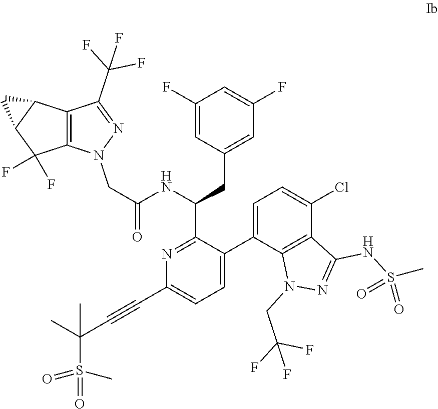 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 3000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 3000 mg of the free acid form of the compound of Formula Ib, as two intramuscular injections of about 3 mL each, each injection comprising about 1500 mg of the free acid form of the compound of Formula Ib, once every twelve months from the first day, wherein each intramuscular injection comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

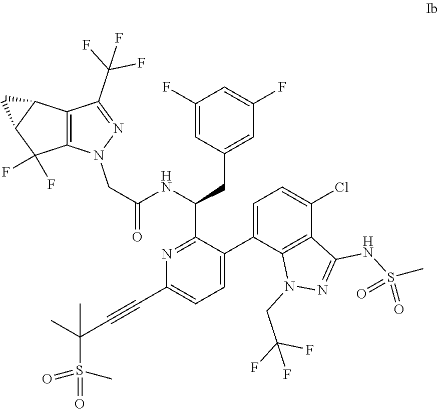 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 30% to about 45% of the free acid form of the compound of Formula Ib;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

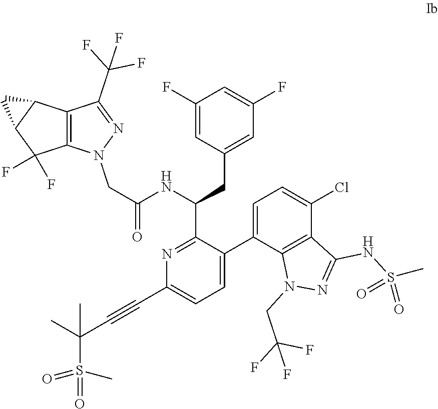 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

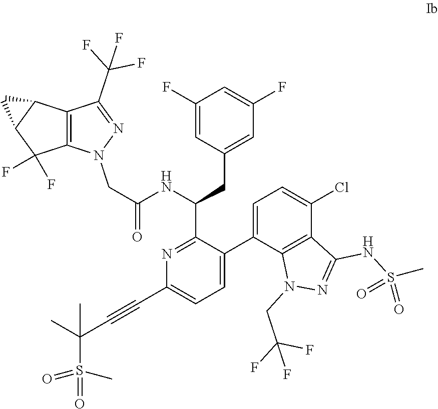 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

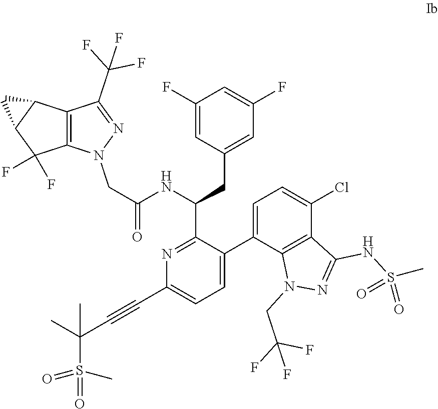 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every six months from the first day, wherein the composition comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

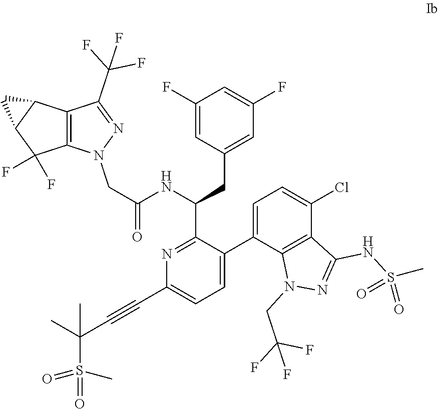 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1500 mg of the free acid form of the compound of Formula Ib, as one intramuscular injection of about 3 mL, every six months from the first day, wherein the intramuscular injection comprises:
   about 30% to about 45% of the free acid form of the compound of Formula Ib;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

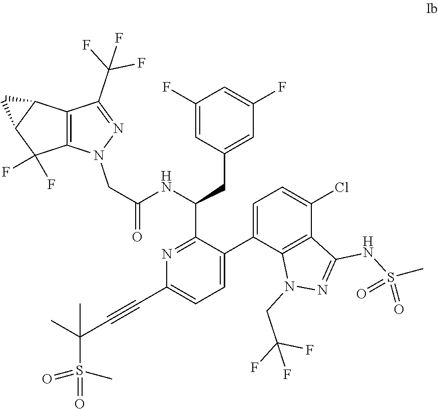 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1500 mg of the free acid form of the compound of Formula Ib, as one intramuscular injection of about 3 mL, every six months from the first day, wherein the intramuscular injection comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

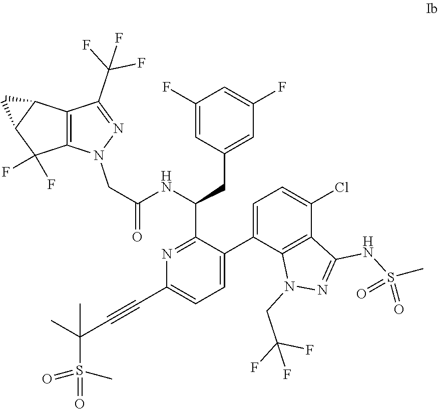 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1500 mg of the free acid form of the compound of Formula Ib, as one intramuscular injection of about 3 mL, every six months from the first day, wherein the intramuscular injection comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

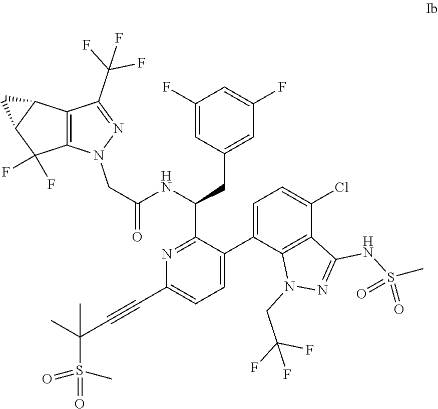 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1500 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1500 mg of the free acid form of the compound of Formula Ib, as one intramuscular injection of about 3 mL, every six months from the first day, wherein the intramuscular injection comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

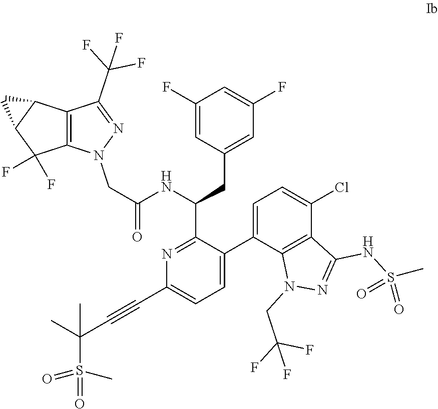 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 30% to about 45% of the free acid form of the compound of Formula Ib;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

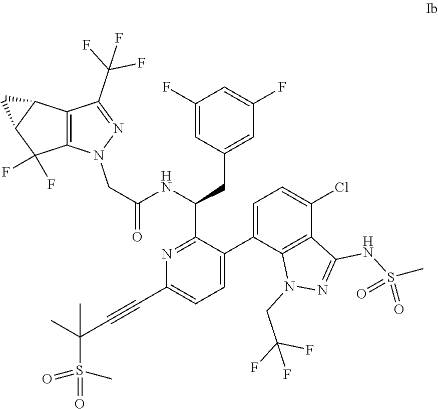 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

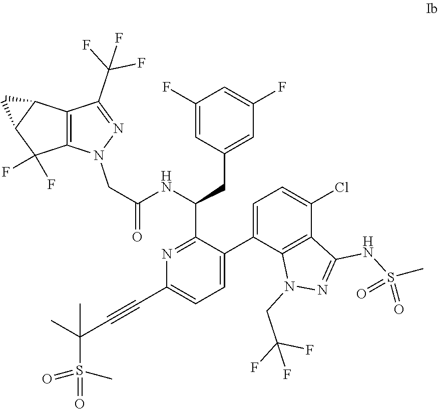 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

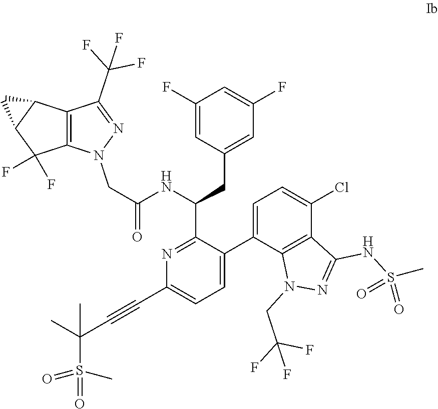 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient a pharmaceutical composition comprising a free acid form of the compound of Formula Ib, PEG 300, ethanol, and water, once every four months from the first day, wherein the composition comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

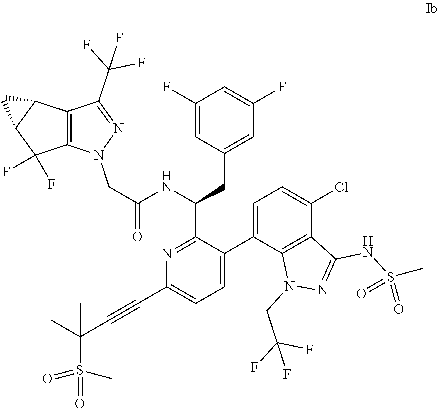 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1000 mg of the free acid form of the compound of Formula Ib, as one intramuscular injection of about 2 mL, every four months from the first day, wherein the intramuscular injection comprises:
   about 30% to about 45% of the free acid form of the compound of Formula Ib;
   about 40% to about 50% of the PEG 300;
   about 7% to about 15% of the ethanol; and
   about 5% to about 10% of the water.

In some embodiments, the present disclosure relates to a method of reducing the risk of sexually acquired HIV in a patient, comprising administering to the patient a compound of

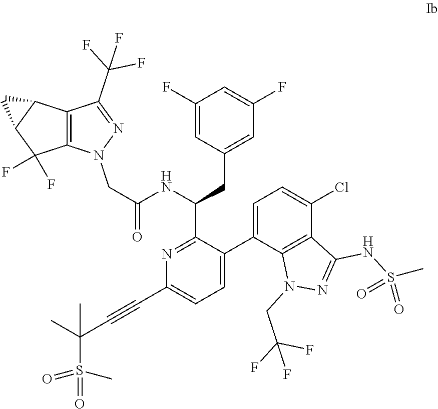 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1000 mg of the free acid form of the compound of Formula Ib, as one intramuscular injection of about 2 mL, every four months from the first day, wherein the intramuscular injection comprises:
   about 41% to about 43% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

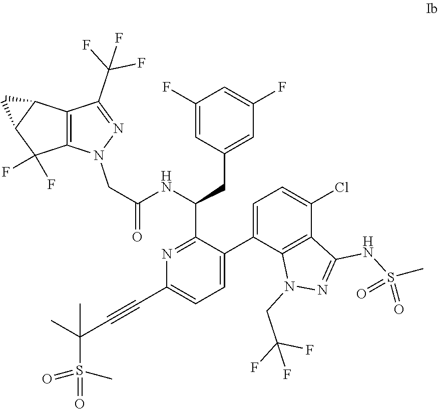 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1000 mg of the free acid form of the compound of Formula Ib, as one intramuscular injection of about 2 mL, every four months from the first day, wherein the intramuscular injection comprises:
   about 42% of the free acid form of the compound of Formula Ib;
   about 37% to about 43% of the PEG 300;
   about 9% to about 11% of the ethanol; and
   about 6% to about 8% of the water.

In some embodiments, the present disclosure relates to a method of reducing the risk of sexually acquired HIV in a patient, comprising administering to the patient a compound of

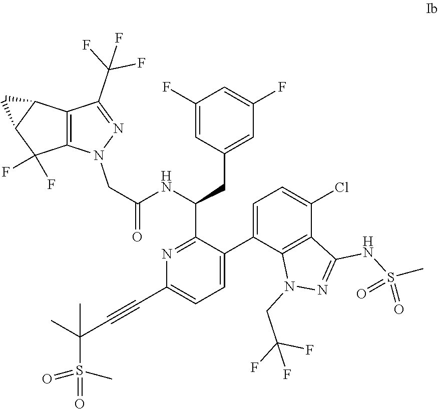 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) administering to the patient an initiation dosage comprising:
   intramuscularly administering to the patient about 1000 mg of the compound of Formula Ib, or a pharmaceutically acceptable salt thereof, on the first day;
   orally administering to the patient about 600 mg of the compound of Formula Ib, or a pharmaceutically acceptable salt thereof, on the first day; and
   orally administering to the patient about 600 mg of the compound of Formula Ib, or a pharmaceutically acceptable salt thereof, on the second day; and
(ii) intramuscularly administering to the patient about 1000 mg of the free acid form of the compound of Formula Ib, as one intramuscular injection of about 2 mL, every four months from the first day, wherein the intramuscular injection comprises:
   about 42.02% of the free acid form of the compound of Formula Ib;
   about 37% to about 43% of the PEG 300;
   about 9% to about 11% of the ethanol; and
   about 6% to about 8% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 30% to about 45% of the free acid form of the compound of Formula Ib;
about 40% to about 50% of the PEG 300;
about 7% to about 15% of the ethanol; and
about 5% to about 10% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 40% to about 43% of the free acid form of the compound of Formula Ib;
about 40% to about 42% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 40% to about 43% of the free acid form of the compound of Formula Ib;
about 40% to about 42% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 42% to about 43% of the free acid form of the compound of Formula Ib;
about 40% to about 41% of the PEG 300;
about 9% to about 11% of the ethanol; and
about 7% to about 8% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 41% to about 43% of the free acid form of the compound of Formula Ib;
about 37% to about 43% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 42% of the free acid form of the compound of Formula Ib;
about 37% to about 43% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 42.02% of the free acid form of the compound of Formula Ib;
about 37% to about 43% of the PEG 300;
about 9% to about 11% of the ethanol; and
about 6% to about 8% of the water.

In some embodiments of the previous embodiments, the intramuscular injection of step (ii) (i.e., the pharmaceutical composition comprising a free acid form of the compound of Formula Ib, PEG 300, ethanol, and water) comprises:
about 42.02% of the free acid form of the compound of Formula Ib;
about 40.78% of the PEG 300;
about 10% of the ethanol; and
about 7.20% of the water.

In some embodiments of the previous embodiments, the intramuscular injection of step (ii) (i.e., the pharmaceutical composition comprising a free acid form of the compound of Formula Ib, PEG 300, ethanol, and water) comprises administering to the patient two intramuscular injections of about 3 mL each, once every twelve months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 3000 mg of the free acid form of the compound of Formula Ib, once every twelve months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 3000 mg of the free acid form of the compound of Formula Ib, as two intramuscular injections of about 1500 mg, once every twelve months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 3000 mg of the free acid form of the compound of Formula Ib, as two 3 mL intramuscular injections of about 1500 mg each, once every twelve months from the first day.

In some embodiments of the previous embodiments, the intramuscular injection of step (ii) (i.e., the pharmaceutical composition comprising a free acid form of the compound of Formula Ib, PEG 300, ethanol, and water) comprises administering to the patient one intramuscular injection of about 3 mL, once every six months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 1500 mg of the free acid form of the compound of Formula Ib, once every six months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 1500 mg of the free acid form of the compound of Formula Ib, as one intramuscular injection, once every six months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 1500 mg of the free acid form of the compound of Formula Ib, as one 3 mL intramuscular injection, once every six months from the first day.

In some embodiments of the previous embodiments, the intramuscular injection of step (ii) (i.e., the pharmaceutical composition comprising a free acid form of the compound of Formula Ib, PEG 300, ethanol, and water) comprises administering to the patient one intramuscular injection of about 2 mL, once every four months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 1000 mg of the free acid form of the compound of Formula Ib, once every four months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 1000 mg of the free acid form of the compound of Formula Ib, as one intramuscular injection, once every four months from the first day.

In some embodiments of the previous embodiments, the intramuscular injection of step (ii) (i.e., the pharmaceutical composition comprising a free acid form of the compound of Formula Ib, PEG 300, ethanol, and water) comprises intramuscularly administering to the patient about 1000 mg of the free acid form of the compound of Formula Ib, as one 2 mL intramuscular injection, once every four months from the first day.

In some embodiments, methods for reducing the risk of acquiring HIV (e.g., HIV-1 and/or HIV-2) comprise administration of a composition provided herein to a patient in combination with safer sexual intercourse practices. In certain embodiments, methods for reducing the risk of acquiring HIV (e.g., HIV-1 and/or HIV-2) comprise administration of a composition provided herein to a patient at risk of acquiring HIV. Examples of patients at high risk for acquiring HIV include, without limitation, a patient who is at risk of sexual transmission of HIV.

In some embodiments, the reduction in risk of acquiring HIV is at least about 40%, 50%, 60%, 70%, 80%, 90%, or 95% (compared to a patient having not been administered the compound of Formula Ia or Ib, or a pharmaceutically acceptable salt thereof according to any of the methods provided herein). In some embodiments, the reduction in risk of acquiring HIV is about 80%, 85%, or 90%. In some embodiments, the reduction in risk of acquiring HIV is at least about 75%. In some embodiments, the reduction in risk of acquiring HIV is at least about 80%. In some embodiments, the reduction in risk of acquiring HIV is at least about 85%. In some embodiments, the reduction in risk of acquiring HIV is at least about 90%.

Administration of the compound of Formula Ia or Ib, or a pharmaceutically acceptable salt or free acid form thereof, can be carried out via any of the accepted modes of administration of agents for serving similar utilities. The pharmaceutical compositions of the disclosure can be prepared by combining the compound of Formula Ia or Ib, or a pharmaceutically acceptable salt or free acid form thereof, with an appropriate pharmaceutically acceptable carrier and, in specific embodiments, the compositions are formulated into preparations in solid, semi solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral, sublingual, buccal, rectal, vaginal, and intranasal. In some embodiments, pharmaceutical compositions of the disclosure are injectable compositions (e.g., intramuscular (IM) or intraperitoneal (IP)).

In some embodiments, a composition provided herein (e.g., a composition comprising a compound of Formula Ia or Ib, or a pharmaceutically acceptable salt or free acid form thereof) is administered parenterally. Parenteral administration includes, but is not limited to, intravenous, intraarterial, subcutaneous, intraperitoneal, intramuscular, intracranial, transdermal, and vaginal administration. Parenteral administration can be for example, in the form of a single bolus dose or by a continuous perfusion pump. In some embodiments, a composition provided herein is administered to the patient through a medical device. Exemplary medical devices include, but are not limited to, a patch (e.g., a transdermal patch), an implantable device (e.g., an implantable device for metered or sustained release of an active agent; a subdermal device), a syringe, a contraceptive device (e.g., a vaginal ring, an intrauterine device), and the like. In some embodiments, the medical device is a syringe.

In some embodiments, formulations suitable for parenteral administration (for example, intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and can be found, for example, in the Handbook of Pharmaceutical Excipients (eds. Rowe, Sheskey & Quinn), 6th edition 2009.

In some embodiments, a composition provided herein (e.g., a composition comprising a compound of Formula Ia or Ib, or a pharmaceutically acceptable salt or free acid form thereof, PEG 300, water, and ethanol), is suitable for parenteral administration (for example, an SC or IM formulation).

In some embodiments, a composition provided herein (e.g., a composition comprising a compound of Formula Ia or Ib, or a pharmaceutically acceptable salt or free acid form thereof, PEG 300, water, and ethanol), is suitable for intramuscular administration.

In some embodiments, a composition provided herein (e.g., a composition comprising a compound of Formula Ia or Ib, or a pharmaceutically acceptable salt or free acid form thereof, PEG 300, water, and ethanol), is suitable for subcutaneous administration.

In some embodiments, the compositions provided herein do not comprise a poloxamer. In some embodiments, the compositions provided herein do not comprise poloxamer 188.

In some embodiments of the methods provided herein, a composition provided herein is administered to the patient as a solution. In some embodiments of the methods provided herein, a composition provided herein is administered to the patient as a solution for injection (e.g., for subcutaneous or intramuscular administration). In some embodiments of the methods provided herein, a composition provided herein is administered to the patient as a solution for subcutaneous administration. In some embodiments of the methods provided herein, a composition provided herein is administered to the patient as a solution for intramuscular administration. In certain embodiments, a composition provided herein is a parenteral formulation. In certain embodiments, a composition provided herein is administered subcutaneously to a patient in need thereof. In certain embodiments, a composition provided herein is administered intramuscularly to a patient in need thereof.

In some embodiments, the compound of Formula Ia or Ib, or a pharmaceutically acceptable salt thereof, is administered subcutaneously at a concentration of about 10 mg/mL to about 600 mg/mL. In some embodiments, the compound of Formula Ia or Ib, or a pharmaceutically acceptable salt thereof, is administered subcutaneously at a concentration of about 10 mg/mL to about 500 mg/mL. In some embodiments, the compound of Formula Ia or Ib, or a pharmaceutically acceptable salt thereof, is administered subcutaneously at a concentration of about 50 mg/mL. In some embodiments, the compound of Formula Ia or Ib, or a pharmaceutically acceptable salt thereof, is administered subcutaneously at a concentration of about 100 mg/mL. In some embodiments, the compound of Formula Ia or Formula Ib, or a pharmaceutically acceptable salt thereof, is administered subcutaneously at a concentration of about 125 mg/mL. In some embodiments, the compound of Formula Ia or Ib, or a pharmaceutically acceptable salt thereof, is administered subcutaneously at a concentration of about 150 mg/mL. In some embodiments, the compound of Formula Ia or Formula Ib, or a pharmaceutically acceptable salt thereof, is administered subcutaneously at a concentration of about 175 mg/mL. In some embodiments, the compound of Formula Ia or Ib, or a pharmaceutically acceptable salt thereof, is administered subcutaneously at a concentration of about 300 mg/mL. In some embodiments, the compound of Formula Ia or Ib, or a pharmaceutically acceptable salt thereof, is administered subcutaneously at a concentration of about 309 mg/mL. In some embodiments, the compound of Formula Ia or Ib, or a pharmaceutically acceptable salt thereof, is administered subcutaneously at a concentration of about 400 mg/mL. In some embodiments, the compound of Formula Ia or Ib, or a pharmaceutically acceptable salt thereof, is administered subcutaneously at a concentration of about 500 mg/mL. In some embodiments, the compound of Formula Ia or Ib, or a pharmaceutically acceptable salt thereof, is administered subcutaneously at a concentration of about 600 mg/mL.

In some embodiments, the compound of Formula Ia or Ib, or a pharmaceutically acceptable salt thereof, is administered intramuscularly at a concentration of about 10 mg/mL to about 600 mg/mL. In some embodiments, the compound of Formula Ia or Ib, or a pharmaceutically acceptable salt thereof, is administered intramuscularly at a concentration of about 10 mg/mL to about 500 mg/mL. In some embodiments, the compound of Formula Ia or Ib, or a pharmaceutically acceptable salt thereof, is administered intramuscularly at a concentration of about 200 mg/mL to about 500 mg/mL. In some embodiments, the compound of Formula Ia or Ib, or a pharmaceutically acceptable salt thereof, is administered intramuscularly at a concentration of about 100 mg/mL. In some embodiments, the compound of Formula Ia or Ib, or a pharmaceutically acceptable salt thereof, is administered intramuscularly at a concentration of about 200 mg/mL. In some embodiments, the compound of Formula Ia or Ib, or a pharmaceutically acceptable salt thereof, is administered intramuscularly at a concentration of about 300 mg/mL. In some embodiments, the compound of Formula Ia or Ib, or a pharmaceutically acceptable salt thereof, is administered intramuscularly at a concentration of about 400 mg/mL. In some embodiments, the compound of Formula Ia or Ib, or a pharmaceutically acceptable salt thereof, is administered intramuscularly at a concentration of about 500 mg/mL. In some embodiments, the compound of Formula Ia or Ib, or a pharmaceutically acceptable salt thereof, is administered intramuscularly at a concentration of about 600 mg/mL.

In some embodiments, about 800 mg to about 6000 mg of the compound of Formula Ia or Ib, or a pharmaceutically acceptable salt thereof, is administered to a patient in need thereof, for example, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, or about 6000 mg.

In some embodiments, about 2000 mg to about 6000 mg of the compound of Formula Ia or Ib, or a pharmaceutically acceptable salt thereof, is administered to a patient in need thereof, for example, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, or about 6000 mg.

In some embodiments, about 800 mg to about 6000 mg of a sodium salt of the compound of Formula Ia or Ib, is administered to a patient in need thereof, for example, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, or about 6000 mg.

In some embodiments, about 2000 mg to about 6000 mg of a sodium salt of the compound of Formula Ia or Ib, is administered to a patient in need thereof, for example, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, or about 6000 mg.

In some embodiments, about 800 mg to about 6000 mg of a free acid form of the compound of Formula Ia or Ib, is administered to a patient in need thereof, for example, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, or about 6000 mg.

In some embodiments, about 2000 mg to about 6000 mg of a free acid form of the compound of Formula Ia or Ib, is administered to a patient in need thereof, for example, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, or about 6000 mg.

In some embodiments, about 4500 mg to about 5500 mg of the compound of Formula Ia or Ib, or a pharmaceutically acceptable salt thereof, is administered to a patient in need thereof. In some embodiments, about 4900 mg to about 5100 mg of the compound of Formula Ia or Ib, or a pharmaceutically acceptable salt thereof, is administered to a patient in need thereof. In some embodiments, about 4950 mg to about 5050 mg of the compound of Formula Ia or Ib, or a pharmaceutically acceptable salt thereof, is administered to a patient in need thereof. In some embodiments, about 5000 mg of the compound of Formula Ia or Ib, or a pharmaceutically acceptable salt thereof, is administered to a patient in need thereof.

In some embodiments, about 4500 mg to about 5500 mg of a sodium salt of the compound of Formula Ia or Ib is administered to a patient in need thereof. In some embodiments, about 4900 mg to about 5100 mg of a sodium salt of the compound of Formula Ia or Ib is administered to a patient in need thereof. In some embodiments, about 4950 mg to about 5050 mg of a sodium salt of the compound of Formula Ia or Ib is administered to a patient in need thereof. In some embodiments, about 5000 mg of a sodium salt of the compound of Formula Ia or Ib is administered to a patient in need thereof.

In some embodiments, about 4500 mg to about 5500 mg of a sodium salt of the compound of Formula Ia or Ib is intramuscularly administered to a patient in need thereof. In some embodiments, about 4900 mg to about 5100 mg of a sodium salt of the compound of Formula Ia or Ib is intramuscularly administered to a patient in need thereof. In some embodiments, about 4950 mg to about 5050 mg of a sodium salt of the compound of Formula Ia or Ib is intramuscularly administered to a patient in need thereof. In some embodiments, about 5000 mg of a sodium salt of the compound of Formula Ia or Ib is intramuscularly administered to a patient in need thereof.

In some embodiments, about 4500 mg to about 5500 mg of a free acid form of the compound of Formula Ia or Ib is administered to a patient in need thereof. In some embodiments, about 4900 mg to about 5100 mg of a free acid form of the compound of Formula Ia or Ib is administered to a patient in need thereof. In some embodiments, about 4950 mg to about 5050 mg of a free acid form of the compound of Formula Ia or Ib is administered to a patient in need thereof. In some embodiments, about 5000 mg of a free acid form of the compound of Formula Ia or Ib is administered to a patient in need thereof.

In some embodiments, about 4500 mg to about 5500 mg of a free acid form of the compound of Formula Ia or Ib is intramuscularly administered to a patient in need thereof. In some embodiments, about 4900 mg to about 5100 mg of a free acid form of the compound of Formula Ia or Ib is intramuscularly administered to a patient in need thereof. In some embodiments, about 4950 mg to about 5050 mg of a free acid form of the compound of Formula Ia or Ib is intramuscularly administered to a patient in need thereof. In some embodiments, about 5000 mg of a free acid form of the compound of Formula Ia or Ib is intramuscularly administered to a patient in need thereof.

In some embodiments, about 3000 mg to about 3500 mg of the compound of Formula Ia or Ib, or a pharmaceutically acceptable salt thereof, is administered to a patient in need thereof. In some embodiments, about 3100 mg to about 3300 mg of the compound of Formula Ia or Ib, or a pharmaceutically acceptable salt thereof, is administered to a patient in need thereof. In some embodiments, about 3150 mg to about 3250 mg of the compound of Formula Ia or Ib, or a pharmaceutically acceptable salt thereof, is administered to a patient in need thereof. In some embodiments, about 3200 mg of the compound of Formula Ia or Ib, or a pharmaceutically acceptable salt thereof, is administered to a patient in need thereof.

In some embodiments, about 3000 mg to about 3500 mg of a sodium salt of Formula Ia or Ib is administered to a patient in need thereof. In some embodiments, about 3100 mg to about 3300 mg of a sodium salt of Formula Ia or Ib is administered to a patient in need thereof. In some embodiments, about 3150 mg to about 3250 mg of a sodium salt of Formula Ia or Ib is administered to a patient in need thereof. In some embodiments, about 3200 mg of a sodium salt of Formula Ia or Ib is administered to a patient in need thereof.

In some embodiments, about 3000 mg to about 3500 mg of a sodium salt of Formula Ia or Ib is subcutaneously administered to a patient in need thereof. In some embodiments, about 3100 mg to about 3300 mg of a sodium salt of Formula Ia or Ib is subcutaneously administered to a patient in need thereof. In some embodiments, about 3150 mg to about 3250 mg of a sodium salt of Formula Ia or Ib is subcutaneously administered to a patient in need thereof. In some embodiments, about 3200 mg of a sodium salt of Formula Ia or Ib is subcutaneously administered to a patient in need thereof.

In some embodiments, about 2200 mg to about 3200 mg of a free acid form of the compound of Formula Ia or Ib is administered to a patient in need thereof. In some embodiments, about 2200 mg to about 3200 mg of a free acid form of the compound of Formula Ia or Ib is subcutaneously administered to a patient in need thereof. In some embodiments, about 2300 mg to about 2500 mg of a free acid form of the compound of Formula Ia or Ib is administered to a patient in need thereof. In some embodiments, about 2350 mg to about 2450 mg of a free acid form of the compound of Formula Ia or Ib is administered to a patient in need thereof. In some embodiments, about 2400 mg of a free acid form of the compound of Formula Ia or Ib is administered to a patient in need thereof. In some embodiments, about 2300 mg to about 2500 mg of a free acid form of the compound of Formula Ia or Ib is subcutaneously administered to a patient in need thereof. In some embodiments, about 2350 mg to about 2450 mg of a free acid form of the compound of Formula Ia or Ib is subcutaneously administered to a patient in need thereof. In some embodiments, about 2400 mg of a free acid form of the compound of Formula Ia or Ib is subcutaneously administered to a patient in need thereof. In some embodiments, about 2900 mg to about 3100 mg of a free acid form of the compound of Formula Ia or Ib is administered to a patient in need thereof. In some embodiments, about 2950 mg to about 3050 mg of a free acid form of the compound of Formula Ia or Ib is administered to a patient in need thereof. In some embodiments, about 3000 mg of a free acid form of the compound of Formula Ia or Ib is administered to a patient in need thereof. In some embodiments, about 2900 mg to about 3100 mg of a free acid form of the compound of Formula Ia or Ib is subcutaneously administered to a patient in need thereof. In some embodiments, about 2950 mg to about 3050 mg of a free acid form of the compound of Formula Ia or Ib is subcutaneously administered to a patient in need thereof. In some embodiments, about 3000 mg of a free acid form of the compound of Formula Ia or Ib is subcutaneously administered to a patient in need thereof.

In some embodiments, about 800 mg to about 2000 mg of the compound of Formula Ia or Ib, or a pharmaceutically acceptable salt thereof, is administered to a patient in need thereof. In some embodiments, about 1200 mg to about 1800 mg of the compound of Formula Ia or Ib, or a pharmaceutically acceptable salt thereof, is administered to a patient in need thereof. In some embodiments, about 1400 mg to about 1600 mg of the compound of Formula Ia or Ib, or a pharmaceutically acceptable salt thereof, is administered to a patient in need thereof. In some embodiments, about 1450 mg to about 1550 mg of the compound of Formula Ia or Ib, or a pharmaceutically acceptable salt thereof, is administered to a patient in need thereof. In some embodiments, about 1500 mg of the compound of Formula Ia or Ib, or a pharmaceutically acceptable salt thereof, is administered to a patient in need thereof.

In some embodiments, about 800 mg to about 2000 mg of the compound of Formula Ia or Ib, or a pharmaceutically acceptable salt thereof, is intramuscularly administered to a patient in need thereof. In some embodiments, about 1200 mg to about 1800 mg of the compound of Formula Ia or Ib, or a pharmaceutically acceptable salt thereof, is intramuscularly administered to a patient in need thereof. In some embodiments, about 1400 mg to about 1600 mg of the compound of Formula Ia or Ib, or a pharmaceutically acceptable salt thereof, is intramuscularly administered to a patient in need thereof. In some embodiments, about 1450 mg to about 1550 mg of the compound of Formula Ia or Ib, or a pharmaceutically acceptable salt thereof, is intramuscularly administered to a patient in need thereof. In some embodiments, about 1500 mg of the compound of Formula Ia or Ib, or a pharmaceutically acceptable salt thereof, is intramuscularly administered to a patient in need thereof.

In some embodiments, about 800 mg to about 2000 mg of the compound of Formula Ia or Ib, or a pharmaceutically acceptable salt thereof, is administered to a patient in need thereof. In some embodiments, about 1000 mg to about 2000 mg of the compound of Formula Ia or Ib, or a pharmaceutically acceptable salt thereof, is administered to a patient in need thereof. In some embodiments, about 1200 mg to about 1800 mg of the compound of Formula Ia or Ib, or a pharmaceutically acceptable salt thereof, is administered to a patient in need thereof. In some embodiments, about 1400 mg to about 1600 mg of the compound of Formula Ia or Ib, or a pharmaceutically acceptable salt thereof, is administered to a patient in need thereof. In some embodiments, about 1450 mg to about 1550 mg of the compound of Formula Ia or Ib, or a pharmaceutically acceptable salt thereof, is administered to a patient in need thereof. In some embodiments, about 1500 mg of the compound of Formula Ia or Ib, or a pharmaceutically acceptable salt thereof, is administered to a patient in need thereof.

In some embodiments, about 800 mg to about 2000 mg of the compound of Formula Ia or Ib, or a pharmaceutically acceptable salt thereof, is intramuscularly administered to a patient in need thereof. In some embodiments, about 1000 mg to about 2000 mg of the compound of Formula Ia or Ib, or a pharmaceutically acceptable salt thereof, is intramuscularly administered to a patient in need thereof. In some embodiments, about 1200 mg to about 1800 mg of the compound of Formula Ia or Ib, or a pharmaceutically acceptable salt thereof, is intramuscularly administered to a patient in need thereof. In some embodiments, about 1400 mg to about 1600 mg of the compound of Formula Ia or Ib, or a pharmaceutically acceptable salt thereof, is intramuscularly administered to a patient in need thereof. In some embodiments, about 1450 mg to about 1550 mg of the compound of Formula Ia or Ib, or a pharmaceutically acceptable salt thereof, is intramuscularly administered to a patient in need thereof. In some embodiments, about 1500 mg of the compound of Formula Ia or Ib, or a pharmaceutically acceptable salt thereof, is intramuscularly administered to a patient in need thereof.

In some embodiments, about 800 mg to about 1200 mg of the compound of Formula Ia or Ib, or a pharmaceutically acceptable salt thereof, is administered to a patient in need thereof. In some embodiments, about 900 mg to about 1100 mg of the compound of Formula Ia or Ib, or a pharmaceutically acceptable salt thereof, is administered to a patient in need thereof. In some embodiments, about 900 mg to about 1000 mg of the compound of Formula Ia or Ib, or a pharmaceutically acceptable salt thereof, is administered to a patient in need thereof. In some embodiments, about 950 mg to about 1050 mg of the compound of Formula Ia or Ib, or a pharmaceutically acceptable salt thereof, is administered to a patient in need thereof. In some embodiments, about 1000 mg of the compound of Formula Ia or Ib, or a pharmaceutically acceptable salt thereof, is administered to a patient in need thereof.

In some embodiments, about 800 mg to about 1200 mg of the compound of Formula Ia or Ib, or a pharmaceutically acceptable salt thereof, is intramuscularly administered to a patient in need thereof. In some embodiments, about 900 mg to about 1100 mg of the compound of Formula Ia or Ib, or a pharmaceutically acceptable salt thereof, is intramuscularly administered to a patient in need thereof. In some embodiments, about 900 mg to about 1000 mg of the compound of Formula Ia or Ib, or a pharmaceutically acceptable salt thereof, is intramuscularly administered to a patient in need thereof. In some embodiments, about 950 mg to about 1050 mg of the compound of Formula Ia or Ib, or a pharmaceutically acceptable salt thereof, is intramuscularly administered to a patient in need thereof. In some embodiments, about 1000 mg of the compound of Formula Ia or Ib, or a pharmaceutically acceptable salt thereof, is intramuscularly administered to a patient in need thereof.

In some embodiments, a composition provided herein is suitable for administration once every twelve months (i.e., once every year). In some embodiments, a composition provided herein is administered to the patient once every twelve months.

In some embodiments, a composition provided herein is administered intramuscularly to the patient once every twelve months.

In some embodiments, a composition provided herein is administered subcutaneously to the patient once every twelve months.

In some embodiments, the methods provided herein comprise administering to the patient about 5 mL to about 10 mL of a pharmaceutical composition provided herein, for example, about 5 mL, about 6 mL, about 7 mL, about 8 mL, about 9 mL, or about 10 mL, once every twelve months.

In some embodiments, the methods provided herein comprise administering to the patient about 4 mL to about 8 mL of a pharmaceutical composition provided herein, for example, about 4 mL, about 5 mL, about 6 mL, about 7 mL, or about 8 mL, once every twelve months. In some embodiments, the methods provided herein comprise administering to the patient about 6 mL of a pharmaceutical composition provided herein once every twelve months.

In some embodiments, the method comprises intramuscularly administering to the patient about 10 mL of a pharmaceutical composition provided herein, once every twelve months. In some embodiments, the method comprises two intramuscular injections of about 5 mL each of a composition provided herein, once every twelve months.

In some embodiments, the method comprises intramuscularly administering to the patient about 6 mL of a pharmaceutical composition provided herein, once every twelve months. In some embodiments, the method comprises two intramuscular injections of about 3 mL each of a composition provided herein, once every twelve months.

In some embodiments, the method comprises two intramuscular injections of about 2500 mg of the compound of Formula Ia or Formula Ib, or a pharmaceutically acceptable salt thereof, once every twelve months. In some embodiments, the method comprises two intramuscular injections of about 2500 mg of a sodium salt of the compound of Formula Ia or Formula Ib, once every twelve months. In some embodiments, the method comprises two intramuscular injections of about 2500 mg of a free acid form of the compound of Formula Ia or Formula Ib, once every twelve months.

In some embodiments, the method comprises intramuscularly administering about 5000 mg of the compound of Formula Ia or Formula Ib, or a pharmaceutically acceptable salt thereof, once every twelve months. In some embodiments, the method comprises intramuscularly administering about 5000 mg of a sodium salt of the compound of Formula Ia or Formula Ib, once every twelve months. In some embodiments, the method comprises intramuscularly administering about 5000 mg of the free acid form of the compound of Formula Ia or Formula Ib, once every twelve months.

In some embodiments, the method comprises intramuscularly administering about 5000 mg of the compound of Formula Ia or Formula Ib, or a pharmaceutically acceptable salt thereof, as two intramuscular injections of about 500 mg/mL, once every twelve months. In some embodiments, the method comprises intramuscularly administering about 5000 mg of a sodium salt of the compound of Formula Ia or Formula Ib, as two intramuscular injections of about 500 mg/mL, once every twelve months. In some embodiments, the method comprises intramuscularly administering about 5000 mg of the free acid form of the compound of Formula Ia or Formula Ib, as two intramuscular injections of about 500 mg/mL, once every twelve months.

In some embodiments, the method comprises intramuscularly administering about 5000 mg of the compound of Formula Ia or Formula Ib, as two intramuscular injections of 5 mL, each at a concentration of about 500 mg/mL, once every twelve months. In some embodiments, the method comprises intramuscularly administering about 5000 mg of a sodium salt of the compound of Formula Ia or Formula Ib, as two intramuscular injections of 5 mL, each at a concentration of about 500 mg/mL, once every twelve months. In some embodiments, the method comprises intramuscularly administering about 5000 mg of the free acid form of the compound of Formula Ia or Formula Ib, as two intramuscular injections of 5 mL, each at a concentration of about 500 mg/mL, once every twelve months.

In some embodiments, the method comprises two intramuscular injections of about 1500 mg of the compound of Formula Ia or Formula Ib, or a pharmaceutically acceptable salt thereof, once every twelve months. In some embodiments, the method comprises two intramuscular injections of about 1500 mg of a sodium salt of the compound of Formula Ia or Formula Ib, once every twelve months. In some embodiments, the method comprises two intramuscular injections of about 1500 mg of a free acid form of the compound of Formula Ia or Formula Ib, once every twelve months.

In some embodiments, the method comprises intramuscularly administering about 3000 mg of the compound of Formula Ia or Formula Ib, or a pharmaceutically acceptable salt thereof, once every twelve months. In some embodiments, the method comprises intramuscularly administering about 3000 mg of a sodium salt of the compound of Formula Ia or Formula Ib, once every twelve months. In some embodiments, the method comprises intramuscularly administering about 3000 mg of the free acid form of the compound of Formula Ia or Formula Ib, once every twelve months.

In some embodiments, the method comprises intramuscularly administering about 3000 mg of the compound of Formula Ia or Formula Ib, or a pharmaceutically acceptable salt thereof, as two intramuscular injections of about 500 mg/mL, once every twelve months. In some embodiments, the method comprises intramuscularly administering about 3000 mg of a sodium salt of the compound of Formula Ia or Formula Ib, as two intramuscular injections of about 500 mg/mL, once every twelve months. In some embodiments, the method comprises intramuscularly administering about 3000 mg of the free acid form of the compound of Formula Ia or Formula Ib, as two intramuscular injections of about 500 mg/mL, once every twelve months.

In some embodiments, the method comprises intramuscularly administering about 3000 mg of the compound of Formula Ia or Formula Ib, as two intramuscular injections of 3 mL, each at a concentration of about 500 mg/mL, once every twelve months. In some embodiments, the method comprises intramuscularly administering about 3000 mg of a sodium salt of the compound of Formula Ia or Formula Ib, as two intramuscular injections of 3 mL, each at a concentration of about 500 mg/mL, once every twelve months. In some embodiments, the method comprises intramuscularly administering about 3000 mg of the free acid form of the compound of Formula Ia or Formula Ib, as two intramuscular injections of 3 mL, each at a concentration of about 500 mg/mL, once every twelve months.

In some embodiments the method comprises subcutaneously administering to the patient about 1 mL to about 8 mL of a composition provided herein, once every twelve months, for example, about 1 mL, about 2 mL, about 3 mL, about 4 mL, about 5 mL, about 6 mL, about 7 mL, or about 8 mL.

In some embodiments, the method comprises subcutaneously administering to the patient about 6 mL of a composition provided herein, once every twelve months.

In some embodiments, the method comprises three subcutaneous injections of about 2 mL each of a composition provided herein, once every twelve months.

In some embodiments, the method comprises three or four subcutaneous injections a composition provided herein, each at a concentration of about 400 mg/mL or about 500 mg/mL of the compound of Formula Ia or Formula Ib, or a pharmaceutically acceptable salt or free acid form thereof, once every twelve months. In some embodiments, the method comprises three subcutaneous injections of about 400 mg/mL each, once every twelve months. In some embodiments, the method comprises four subcutaneous injections of about 400 mg/mL each, once every twelve months. In some embodiments, the method comprises three subcutaneous injections of about 500 mg/mL each, once every twelve months.

In some embodiments, the method comprises subcutaneously administering about 2400 mg of the compound of Formula Ia or Formula Ib, or a pharmaceutically acceptable salt thereof, once every twelve months. In some embodiments, the method comprises subcutaneously administering about 2400 mg of a sodium salt of the compound of Formula Ia or Formula Ib, once every twelve months. In some embodiments, the method comprises subcutaneously administering about 2400 mg of the free acid form of the compound of Formula Ia or Formula Ib, once every twelve months.

In some embodiments, the method comprises subcutaneously administering about 2400 mg of the compound of Formula Ia or Formula Ib, or a pharmaceutically acceptable salt thereof, as three subcutaneous injections of about 400 mg/mL, once every twelve months. In some embodiments, the method comprises subcutaneously administering about 2400 mg of a sodium salt of the compound of Formula Ia or Formula Ib, as three subcutaneous injections of about 400 mg/mL, once every twelve months. In some embodiments, the method comprises subcutaneously administering about 2400 mg of the free acid form of the compound of Formula Ia or Formula Ib, as three subcutaneous injections of about 400 mg/mL each, once every twelve months.

In some embodiments, the method comprises subcutaneously administering about 2400 mg of the compound of Formula Ia or Formula Ib, or a pharmaceutically acceptable salt thereof, as three subcutaneous injections of 2 mL each, at a concentration of about 400 mg/mL each, once every twelve months. In some embodiments, the method comprises subcutaneously administering about 2400 mg of a sodium salt of the compound of Formula Ia or Formula Ib, as three subcutaneous injections of 2 mL each, at a concentration of about 400 mg/mL each, once every twelve months. In some embodiments, the method comprises subcutaneously administering about 2400 mg of the free acid form of the compound of Formula Ia or Formula Ib, as three subcutaneous injections of 2 mL each, at a concentration of about 400 mg/mL each, once every twelve months.

In some embodiments, the method comprises subcutaneously administering about 3200 mg of the compound of Formula Ia or Formula Ib, or a pharmaceutically acceptable salt thereof, as four subcutaneous injections of about 400 mg/mL, once every twelve months. In some embodiments, the method comprises subcutaneously administering about 3200 mg of a sodium salt of the compound of Formula Ia or Formula Ib, as four subcutaneous injections of about 400 mg/mL, once every twelve months.

In some embodiments, the method comprises subcutaneously administering about 3200 mg of the compound of Formula Ia or Formula Ib, or a pharmaceutically acceptable salt thereof, as four subcutaneous injections of 2 mL each, at a concentration of about 400 mg/mL each, once every twelve months. In some embodiments, the method comprises subcutaneously administering about 3200 mg of a sodium salt of the compound of Formula Ia or Formula Ib, as four subcutaneous injections of 2 mL each, at a concentration of about 400 mg/mL each, once every twelve months.

In some embodiments, the method comprises subcutaneously administering about 3000 mg of the free acid form of the compound of Formula Ia or Formula Ib, as three subcutaneous injections of about 500 mg/mL each, once every twelve months.

In some embodiments, the method comprises subcutaneously administering about 3000 mg of the free acid form of the compound of Formula Ia or Formula Ib, as three subcutaneous injections of 2 mL each, at a concentration of about 500 mg/mL each, once every twelve months.

In some embodiments of the methods provided herein, a composition provided herein is administered subcutaneously to a patient in need thereof once every twelve months.

In some embodiments of the methods provided herein, a composition comprising:
about 30% to about 45% of the free acid form of the compound of Formula Ia;
about 40% to about 50% of PEG 300;
about 7% to about 15% of ethanol; and
about 5% to about 10% of water;
is administered subcutaneously to a patient in need thereof once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises subcutaneously administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises subcutaneously administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every twelve months. In some embodiments, the above composition consists of the specified ingredients. In some embodiments, the above composition consists essentially of the specified ingredients.

In some embodiments the methods provided herein comprise subcutaneously administering to the patient about 1 mL to about 8 mL of a pharmaceutical composition provided herein, once every twelve months. In some embodiments, the methods provided herein comprise subcutaneously administering to the patient about 6 mL of a pharmaceutical composition provided herein, once every twelve months. In some embodiments, the methods provided herein comprise three subcutaneous injections of about 2 mL each, once every twelve months.

In some embodiments, the methods provided herein comprise three subcutaneous injections of about 800 mg of the free acid form of a compound of Formula Ia or Formula Ib, once every twelve months. In some embodiments, the methods provided herein comprise subcutaneously administering about 2400 mg of the free acid form of a compound of Formula Ia or Formula Ib, once every twelve months. In some embodiments, the methods provided herein comprise subcutaneously administering about 2400 mg of the free acid form of a compound of Formula Ia or Formula Ib, as three subcutaneous injections of about 800 mg, once every twelve months.

In some embodiments, the methods provided herein comprise three subcutaneous injections of about 1000 mg of the free acid form of a compound of Formula Ia or Formula Ib, once every 12 months. In some embodiments, the methods provided herein comprise subcutaneously administering about 3000 mg of the free acid form of a compound of Formula Ia or Formula Ib, as three subcutaneous injections of about 1000 mg, once every twelve months. In some embodiments, the methods provided herein comprise subcutaneously administering about 3000 mg of the free acid form of a compound of Formula Ia or Formula Ib, once every twelve months.

In some embodiments, the compositions provided herein comprise about 300 mg/mL to about 600 mg/mL of the free acid form of a compound of Formula Ia or Formula Ib. In some embodiments, the compositions provided herein comprise about 400 mg/mL to about 500 mg/mL of the free acid form of a compound of Formula Ia or Formula Ib. In some embodiments, the compositions provided herein comprise about 500 mg/mL of the free acid form of a compound of Formula Ia or Formula Ib. In some embodiments, the compositions provided herein comprise about 400 mg/mL of a free acid form of the compound of Formula Ia or Formula Ib.

In some embodiments of the methods provided herein, a composition provided herein is administered intramuscularly to a patient in need thereof once every twelve months.

In some embodiments of the methods provided herein, a composition comprising
about 30% to about 45% of the free acid form of the compound of Formula Ia;
about 40% to about 50% of PEG 300;
about 7% to about 15% of ethanol; and
about 5% to about 10% of water;
is administered intramuscularly to a patient in need thereof once every twelve months. In some embodiments, the above composition consists of the specified ingredients. In some embodiments, the above composition consists essentially of the specified ingredients.

In some embodiments of the methods provided herein, a composition comprising:
about 41% to about 43% of the free acid form of the compound of Formula Ia;
about 37% to about 43% of PEG 300;
about 9% to about 11% of ethanol; and
about 6% to about 8% of water;
is administered intramuscularly to a patient in need thereof once every twelve months. In some embodiments, the above composition consists of the specified ingredients. In some embodiments, the above composition consists essentially of the specified ingredients.

In some embodiments of the methods provided herein, a composition comprising:
about 42% of the free acid form of the compound of Formula Ia;
about 37% to about 43% of PEG 300;
about 9% to about 11% of ethanol; and
about 6% to about 8% of water;
is administered intramuscularly to a patient in need thereof once every twelve months. In some embodiments, the above composition consists of the specified ingredients. In some embodiments, the above composition consists essentially of the specified ingredients.

In some embodiments of the methods provided herein, a composition comprising:
about 42.02% of the free acid form of the compound of Formula Ia;
about 37% to about 43% of PEG 300;
about 9% to about 11% of ethanol; and
about 6% to about 8% of water;
is administered intramuscularly to a patient in need thereof once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every twelve months. In some embodiments, the above composition consists of the specified ingredients. In some embodiments, the above composition consists essentially of the specified ingredients.

In some embodiments of the methods provided herein, a composition comprising:
about 30% to about 45% of the free acid form of the compound of Formula Ia;
about 40% to about 50% of PEG 300;
about 7% to about 15% of ethanol; and
about 5% to about 10% of water;
is administered intramuscularly to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments of the methods provided herein, a composition comprising:
about 41% to about 43% of the free acid form of the compound of Formula Ia;
about 37% to about 43% of PEG 300;
about 9% to about 11% of ethanol; and
about 6% to about 8% of water;
is administered intramuscularly to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments of the methods provided herein, a composition comprising:
about 42% of the free acid form of the compound of Formula Ia;
about 37% to about 43% of PEG 300;
about 9% to about 11% of ethanol; and
about 6% to about 8% of water;
is administered intramuscularly to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments of the methods provided herein, a composition comprising:
about 42.02% of the free acid form of the compound of Formula Ia;
about 37% to about 43% of PEG 300;
about 9% to about 11% of ethanol; and
about 6% to about 8% of water;
is administered intramuscularly to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every twelve months, wherein the method comprises administering about 3000 mg of the free acid form of the compound of Formula Ia, as two intramuscular injection of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every twelve months, wherein the method comprises administering about 3000 mg of the free acid form of the compound of Formula Ia, as two intramuscular injections of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every twelve months, wherein the method comprises administering about 3000 mg of the free acid form of the compound of Formula Ia, as two intramuscular injections of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every twelve months, wherein the method comprises administering about 3000 mg of the free acid form of the compound of Formula Ia, as two intramuscular injections of 3 mL each, at a concentration of about 500 mg/mL, once every twelve months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every twelve months, wherein the method comprises administering about 3000 mg of the free acid form of the compound of Formula Ia, as two intramuscular injections of 3 mL each, at a concentration of about 500 mg/mL, once every twelve months. In some embodiments, the above composition consists of the specified ingredients. In some embodiments, the above composition consists essentially of the specified ingredients.

In some embodiments, the methods provided herein comprise intramuscularly administering to the patient about 5 mL to about 10 mL of a pharmaceutical composition provided herein, once every twelve months. In some embodiments the methods provided herein comprise intramuscularly administering to the patient about 10 mL of a pharmaceutical composition provided herein, once every twelve months. In some embodiments, the methods provided herein comprise two intramuscular injections of about 5 mL each, once every twelve months.

In some embodiments the methods provided herein comprise intramuscularly administering to the patient about 6 mL of a pharmaceutical composition provided herein, once every twelve months. In some embodiments, the methods provided herein comprise two intramuscular injections of about 3 mL each, once every twelve months.

In some embodiments, the methods provided herein comprise two intramuscular injections of about 2500 mg of the free acid form of a compound of Formula Ia or Formula Ib, once every twelve months. In some embodiments, the methods provided herein comprise intramuscularly administering about 5000 mg of the free acid form of a compound of Formula Ia or Formula Ib, once every twelve months. In some embodiments, the methods provided herein comprise intramuscularly administering about 5000 mg of the free acid form of a compound of Formula Ia or Formula Ib, as two intramuscular injections of about 2500 mg, once every twelve months.

In some embodiments, the methods provided herein comprise two intramuscular injections of about 1500 mg of the free acid form of a compound of Formula Ia or Formula Ib, once every twelve months. In some embodiments, the methods provided herein comprise intramuscularly administering about 3000 mg of the free acid form of a compound of Formula Ia or Formula Ib, once every twelve months. In some embodiments, the methods provided herein comprise intramuscularly administering about 3000 mg of the free acid form of a compound of Formula Ia or Formula Ib, as two intramuscular injections of about 1500 mg, once every twelve months.

In some embodiments, the methods provided herein comprise administering about 3000 mg of the compound of Formula Ia or Formula Ib, or a pharmaceutically acceptable salt thereof, as two intramuscular injections of about 3 mL each, once every twelve months. In some embodiments, the methods provided herein comprise administering about 3000 mg of the compound of Formula Ia or Formula Ib, or a pharmaceutically acceptable salt thereof, as two intramuscular injections of about 3 mL each, each comprising about 1500 mg of the compound of Formula Ia or Formula Ib, or a pharmaceutically acceptable salt thereof, once every twelve months.

In some embodiments, the methods provided herein comprise administering about 3000 mg of the free acid form of the compound of Formula Ia or Formula Ib, as two intramuscular injections of about 3 mL each, each comprising about 1500 mg of the free acid form of the Formula Ia or Formula Ib, once every twelve months. In some embodiments, the methods provided herein comprise administering about 3000 mg of the free acid form of the compound Formula Ib, as two intramuscular injections of about 3 mL each, each comprising about 1500 mg of the free acid form of the Formula Ib, once every twelve months.

As shown in the examples and the figures, Composition 3 in Table 1 is particularly effective at maintaining high plasma concentrations of lenacapavir for a long period of time following its administration (e.g. for over 12 months following its administration). Therefore, the disclosure provides a pharmaceutical composition comprising a free acid form of the compound of Formula Ia:

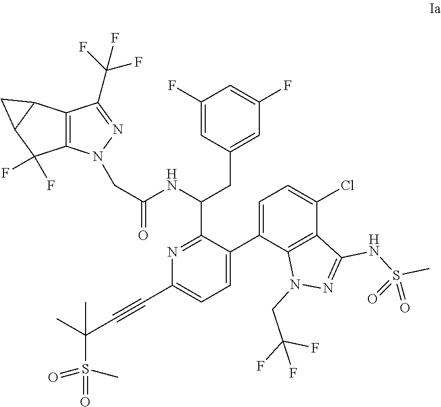 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

PEG 300, ethanol, and water, wherein the composition comprises:
about 30% to about 45% of the free acid form of the compound of Formula Ia;
about 37% to about 50% of the PEG 300;
about 7% to about 15% of the ethanol; and
about 5% to about 10% of the water;
for use in a method of treating or preventing HIV in a patient, wherein the method comprises intramuscular administration of the pharmaceutical composition once every twelve months, once every six months, or once every four months. In some embodiments, the method comprises intramuscular administration of the pharmaceutical composition once every twelve months. In some embodiments, the method comprises intramuscular administration of the pharmaceutical composition once every six months. In some embodiments, the method comprises intramuscular administration of the pharmaceutical composition once every four months. The composition may consist of the ingredients specified above. The composition may be free of poloxamer.

As shown in the examples and the figures, Composition 3 in Table 1 is particularly effective at maintaining high plasma concentrations of lenacapavir for a long period of time following its administration (e.g. for over 12 months following its administration). Therefore, the disclosure provides a pharmaceutical composition comprising a free acid form of the compound of Formula Ib:

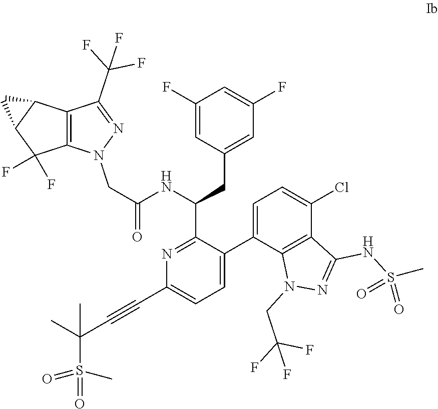 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

PEG 300, ethanol, and water, wherein the composition comprises:
about 30% to about 45% of the free acid form of the compound of Formula Ib;
about 37% to about 50% of the PEG 300;
about 7% to about 15% of the ethanol; and
about 5% to about 10% of the water;
for use in a method of treating or preventing HIV in a patient, wherein the method comprises intramuscular administration of the pharmaceutical composition once every twelve months, once every six months, or once every four months. In some embodiments, the method comprises intramuscular administration of the pharmaceutical composition once every twelve months. In some embodiments, the method comprises intramuscular administration of the pharmaceutical composition once every six months. In some embodiments, the method comprises intramuscular administration of the pharmaceutical composition once every four months. The composition may consist of the ingredients specified above. The composition may be free of poloxamer.

In some embodiments, the methods provided herein comprise administering two intramuscular injections of Composition 3, once every twelve months.

In some embodiments, the methods provided herein comprise administering two intramuscular injections of about 3 mL each of Composition 3, once every twelve months.

In some embodiments, the methods provided herein comprise administering about 3000 mg of the free acid form of the compound of Formula Ia or Formula Ib, as two intramuscular injections of about 3 mL each of Composition 3, once every twelve months.

In some embodiments, the methods provided herein comprise administering about 3000 mg of the free acid form of the compound of Formula Ia, as two intramuscular injections of about 3 mL each of Composition 3, once every twelve months.

In some embodiments, the methods provided herein comprise administering about 3000 mg of the free acid form of the compound of Formula Ib, as two intramuscular injections of about 3 mL each of Composition 3, once every twelve months.

In some embodiments, a composition provided herein is suitable for administration once every six months (i.e., twice every year). In some embodiments, a composition provided herein is administered to the patient once every six months.

In some embodiments, a composition provided herein is administered intramuscularly to the patient once every six months.

In some embodiments, a composition provided herein is administered subcutaneously to the patient once every six months.

In some embodiments, the methods provided herein comprise administering to the patient about 1 mL to about 10 mL of a pharmaceutical composition provided herein, for example, about 1 mL, about 2 mL, about 3 mL, about 4 mL, about 5 mL, about 6 mL, about 7 mL, about 8 mL, about 9 mL, or about 10 mL, once every six months.

In some embodiments, the methods provided herein comprise administering to the patient about 1 mL to about 5 mL of a pharmaceutical composition provided herein, for example, about 1 mL, about 2 mL, about 3 mL, about 4 mL, or about 5 mL, once every six months. In some embodiments, the methods provided herein comprise administering to the patient about 3 mL of a pharmaceutical composition provided herein once every six months.

In some embodiments, the method comprises intramuscularly administering to the patient about 3 mL of a pharmaceutical composition provided herein, once every six months. In some embodiments, the method comprises one intramuscular injection of about 3 mL of a composition provided herein, once every six months.

In some embodiments, the method comprises one intramuscular injection of about 1200 mg of the compound of Formula Ia or Formula Ib, or a pharmaceutically acceptable salt thereof, once every six months. In some embodiments, the method comprises one intramuscular injection of about 1200 mg of a sodium salt of the compound of Formula Ia or Formula Ib, once every six months. In some embodiments, the method comprises one intramuscular injection of about 1200 mg of a free acid form of the compound of Formula Ia or Formula Ib, once every six months.

In some embodiments, the method comprises one intramuscular injection of about 1300 mg of the compound of Formula Ia or Formula Ib, or a pharmaceutically acceptable salt thereof, once every six months. In some embodiments, the method comprises one intramuscular injection of about 1300 mg of a sodium salt of the compound of Formula Ia or Formula Ib, once every six months. In some embodiments, the method comprises one intramuscular injection of about 1300 mg of a free acid form of the compound of Formula Ia or Formula Ib, once every six months.

In some embodiments, the method comprises one intramuscular injection of about 1400 mg of the compound of Formula Ia or Formula Ib, or a pharmaceutically acceptable salt thereof, once every six months. In some embodiments, the method comprises one intramuscular injection of about 1400 mg of a sodium salt of the compound of Formula Ia or Formula Ib, once every six months. In some embodiments, the method comprises one intramuscular injection of about 1400 mg of a free acid form of the compound of Formula Ia or Formula Ib, once every six months.

In some embodiments, the method comprises one intramuscular injection of about 1500 mg of the compound of Formula Ia or Formula Ib, or a pharmaceutically acceptable salt thereof, once every six months. In some embodiments, the method comprises one intramuscular injection of about 1500 mg of a sodium salt of the compound of Formula Ia or Formula Ib, once every six months. In some embodiments, the method comprises one intramuscular injection of about 1500 mg of a free acid form of the compound of Formula Ia or Formula Ib, once every six months.

In some embodiments, the method comprises one intramuscular injection of about 1600 mg of the compound of Formula Ia or Formula Ib, or a pharmaceutically acceptable salt thereof, once every six months. In some embodiments, the method comprises one intramuscular injection of about 1600 mg of a sodium salt of the compound of Formula Ia or Formula Ib, once every six months. In some embodiments, the method comprises one intramuscular injection of about 1600 mg of a free acid form of the compound of Formula Ia or Formula Ib, once every six months.

In some embodiments, the method comprises one intramuscular injection of about 1700 mg of the compound of Formula Ia or Formula Ib, or a pharmaceutically acceptable salt thereof, once every six months. In some embodiments, the method comprises one intramuscular injection of about 1700 mg of a sodium salt of the compound of Formula Ia or Formula Ib, once every six months. In some embodiments, the method comprises one intramuscular injection of about 1700 mg of a free acid form of the compound of Formula Ia or Formula Ib, once every six months.

In some embodiments, the method comprises one intramuscular injection of about 1800 mg of the compound of Formula Ia or Formula Ib, or a pharmaceutically acceptable salt thereof, once every six months. In some embodiments, the method comprises one intramuscular injection of about 1800 mg of a sodium salt of the compound of Formula Ia or Formula Ib, once every six months. In some embodiments, the method comprises one intramuscular injection of about 1800 mg of a free acid form of the compound of Formula Ia or Formula Ib, once every six months.

In some embodiments, the method comprises one intramuscular injection of a composition provided herein, at a concentration of about 500 mg/mL of the compound of Formula Ia or Formula Ib, or a pharmaceutically acceptable salt or free acid form thereof, once every six months. In some embodiments, the method comprises one intramuscular injection of a composition provided herein, at a concentration of about 500 mg/mL of the sodium salt of the compound of Formula Ia or Formula Ib, or a pharmaceutically acceptable salt thereof, once every six months. In some embodiments, the method comprises one intramuscular injection of a composition provided herein, at a concentration of about 500 mg/mL of the free acid form of the compound of Formula Ia or Formula Ib, once every six months.

In some embodiments, the method comprises intramuscularly administering about 1500 mg of the compound of Formula Ia or Formula Ib, or a pharmaceutically acceptable salt thereof, as one intramuscular injection of about 500 mg/mL, once every six months. In some embodiments, the method comprises intramuscularly administering about 1500 mg of a sodium salt of the compound of Formula Ia or Formula Ib, as one intramuscular injection of about 500 mg/mL, once every six months. In some embodiments, the method comprises intramuscularly administering about 1500 mg of the free acid form of the compound of Formula Ia or Formula Ib, as one intramuscular injection of about 500 mg/mL, once every six months.

In some embodiments, the method comprises intramuscularly administering about 1500 mg of the compound of Formula Ia or Formula Ib, as one intramuscular injection of 3 mL, at a concentration of about 500 mg/mL, once every six months. In some embodiments, the method comprises intramuscularly administering about 1500 mg of a sodium salt of the compound of Formula Ia or Formula Ib, as one intramuscular injection of 3 mL, at a concentration of about 500 mg/mL, once every six months. In some embodiments, the method comprises intramuscularly administering about 1500 mg of the free acid form of the compound of Formula Ia or Formula Ib, as one intramuscular injection of 3 mL, at a concentration of about 500 mg/mL, once every six months.

In some embodiments of the methods provided herein, a composition comprising:
about 30% to about 45% of the free acid form of the compound of Formula Ia;
about 40% to about 50% of PEG 300;
about 7% to about 15% of ethanol; and
about 5% to about 10% of water;
is administered intramuscularly to a patient in need thereof once every six months. In some embodiments, the above composition consists of the specified ingredients. In some embodiments, the above composition consists essentially of the specified ingredients.

In some embodiments of the methods provided herein, a composition comprising:
about 41% to about 43% of the free acid form of the compound of Formula Ia;
about 37% to about 43% of PEG 300;
about 9% to about 11% of ethanol; and
about 6% to about 8% of water;
is administered intramuscularly to a patient in need thereof once every six months. In some embodiments, the above composition consists of the specified ingredients. In some embodiments, the above composition consists essentially of the specified ingredients.

In some embodiments of the methods provided herein, a composition comprising:
about 42% of the free acid form of the compound of Formula Ia;
about 37% to about 43% of PEG 300;
about 9% to about 11% of ethanol; and
about 6% to about 8% of water;
is administered intramuscularly to a patient in need thereof once every six months. In some embodiments, the above composition consists of the specified ingredients. In some embodiments, the above composition consists essentially of the specified ingredients.

In some embodiments of the methods provided herein, a composition comprising:
about 42.02% of the free acid form of the compound of Formula Ia;
about 37% to about 43% of PEG 300;
about 9% to about 11% of ethanol; and
about 6% to about 8% of water;
is administered intramuscularly to a patient in need thereof once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every six months. In some embodiments, the above composition consists of the specified ingredients. In some embodiments, the above composition consists essentially of the specified ingredients.

In some embodiments of the methods provided herein, a composition comprising:
about 30% to about 45% of the free acid form of the compound of Formula Ia;
about 40% to about 50% of PEG 300;
about 7% to about 15% of ethanol; and
about 5% to about 10% of water;
is administered intramuscularly to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments of the methods provided herein, a composition comprising:
about 41% to about 43% of the free acid form of the compound of Formula Ia;
about 37% to about 43% of PEG 300;
about 9% to about 11% of ethanol; and
about 6% to about 8% of water;
is administered intramuscularly to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments of the methods provided herein, a composition comprising:
about 42% of the free acid form of the compound of Formula Ia;
about 37% to about 43% of PEG 300;
about 9% to about 11% of ethanol; and
about 6% to about 8% of water;
is administered intramuscularly to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments of the methods provided herein, a composition comprising:
about 42.02% of the free acid form of the compound of Formula Ia;
about 37% to about 43% of PEG 300;
about 9% to about 11% of ethanol; and
about 6% to about 8% of water;
is administered intramuscularly to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every six months, wherein the method comprises administering about 1500 mg of the free acid form of the compound of Formula Ia, as one intramuscular injection of 3 mL, at a concentration of about 500 mg/mL, once every six months. In some embodiments, the above composition consists of the specified ingredients. In some embodiments, the above composition consists essentially of the specified ingredients.

In some embodiments, the methods provided herein comprise administering one intramuscular injection of Composition 3, once every six months.

In some embodiments, the methods provided herein comprise administering one intramuscular injection of about 3 mL of Composition 3, once every six months.

In some embodiments, the methods provided herein comprise administering about 1500 mg of the free acid form of the compound of Formula Ia or Formula Ib, as one intramuscular injection of about 3 mL of Composition 3, once every six months.

In some embodiments, the methods provided herein comprise administering about 1500 mg of the free acid form of the compound of Formula Ia, as one intramuscular injection of about 3 mL of Composition 3, once every six months.

In some embodiments, the methods provided herein comprise administering about 1500 mg of the free acid form of the compound of Formula Ib, as one intramuscular injection of about 3 mL of Composition 3, once every six months.

In some embodiments, a composition provided herein is suitable for administration once every four months (i.e., thrice every year). In some embodiments, a composition provided herein is administered to the patient once every four months.

In some embodiments, a composition provided herein is administered intramuscularly to the patient once every four months.

In some embodiments, a composition provided herein is administered subcutaneously to the patient once every four months.

In some embodiments, the methods provided herein comprise administering to the patient about 1 mL to about 10 mL of a pharmaceutical composition provided herein, for example, about 1 mL, about 2 mL, about 3 mL, about 4 mL, about 5 mL, about 6 mL, about 7 mL, about 8 mL, about 9 mL, or about 10 mL, once every four months.

In some embodiments, the methods provided herein comprise administering to the patient about 1 mL to about 5 mL of a pharmaceutical composition provided herein, for example, about 1 mL, about 2 mL, about 3 mL, about 4 mL, or about 5 mL, once every four months. In some embodiments, the methods provided herein comprise administering to the patient about 2 mL of a pharmaceutical composition provided herein once every four months.

In some embodiments, the method comprises intramuscularly administering to the patient about 2 mL of a pharmaceutical composition provided herein, once every four months. In some embodiments, the method comprises one intramuscular injection of about 2 mL of a composition provided herein, once every four months.

In some embodiments, the method comprises one intramuscular injection of about 700 mg of the compound of Formula Ia or Formula Ib, or a pharmaceutically acceptable salt thereof, once every four months. In some embodiments, the method comprises one intramuscular injection of about 700 mg of a sodium salt of the compound of Formula Ia or Formula Ib, once every four months. In some embodiments, the method comprises one intramuscular injection of about 700 mg of a free acid form of the compound of Formula Ia or Formula Ib, once every four months.

In some embodiments, the method comprises one intramuscular injection of about 800 mg of the compound of Formula Ia or Formula Ib, or a pharmaceutically acceptable salt thereof, once every four months. In some embodiments, the method comprises one intramuscular injection of about 800 mg of a sodium salt of the compound of Formula Ia or Formula Ib, once every four months. In some embodiments, the method comprises one intramuscular injection of about 800 mg of a free acid form of the compound of Formula Ia or Formula Ib, once every four months.

In some embodiments, the method comprises one intramuscular injection of about 900 mg of the compound of Formula Ia or Formula Ib, or a pharmaceutically acceptable salt thereof, once every four months. In some embodiments, the method comprises one intramuscular injection of about 900 mg of a sodium salt of the compound of Formula Ia or Formula Ib, once every four months. In some embodiments, the method comprises one intramuscular injection of about 900 mg of a free acid form of the compound of Formula Ia or Formula Ib, once every four months.

In some embodiments, the method comprises one intramuscular injection of about 1000 mg of the compound of Formula Ia or Formula Ib, or a pharmaceutically acceptable salt thereof, once every four months. In some embodiments, the method comprises one intramuscular injection of about 1000 mg of a sodium salt of the compound of Formula Ia or Formula Ib, once every four months. In some embodiments, the method comprises one intramuscular injection of about 1000 mg of a free acid form of the compound of Formula Ia or Formula Ib, once every four months.

In some embodiments, the method comprises one intramuscular injection of about 1100 mg of the compound of Formula Ia or Formula Ib, or a pharmaceutically acceptable salt thereof, once every four months. In some embodiments, the method comprises one intramuscular injection of about 1100 mg of a sodium salt of the compound of Formula Ia or Formula Ib, once every four months. In some embodiments, the method comprises one intramuscular injection of about 1100 mg of a free acid form of the compound of Formula Ia or Formula Ib, once every four months.

In some embodiments, the method comprises one intramuscular injection of about 1200 mg of the compound of Formula Ia or Formula Ib, or a pharmaceutically acceptable salt thereof, once every four months. In some embodiments, the method comprises one intramuscular injection of about 1200 mg of a sodium salt of the compound of Formula Ia or Formula Ib, once every four months. In some embodiments, the method comprises one intramuscular injection of about 1200 mg of a free acid form of the compound of Formula Ia or Formula Ib, once every four months.

In some embodiments, the method comprises one intramuscular injection of about 1300 mg of the compound of Formula Ia or Formula Ib, or a pharmaceutically acceptable salt thereof, once every four months. In some embodiments, the method comprises one intramuscular injection of about 1300 mg of a sodium salt of the compound of Formula Ia or Formula Ib, once every four months. In some embodiments, the method comprises one intramuscular injection of about 1300 mg of a free acid form of the compound of Formula Ia or Formula Ib, once every four months.

In some embodiments, the method comprises one intramuscular injection of a composition provided herein, at a concentration of about 500 mg/mL of the compound of Formula Ia or Formula Ib, or a pharmaceutically acceptable salt or free acid form thereof, once every four months. In some embodiments, the method comprises one intramuscular injection of a composition provided herein, at a concentration of about 500 mg/mL of the sodium salt of the compound of Formula Ia or Formula Ib, or a pharmaceutically acceptable salt thereof, once every four months. In some embodiments, the method comprises one intramuscular injection of a composition provided herein, at a concentration of about 500 mg/mL of the free acid form of the compound of Formula Ia or Formula Ib, once every four months.

In some embodiments, the method comprises intramuscularly administering about 1000 mg of the compound of Formula Ia or Formula Ib, or a pharmaceutically acceptable salt thereof, as one intramuscular injection of about 500 mg/mL, once every four months. In some embodiments, the method comprises intramuscularly administering about 1000 mg of a sodium salt of the compound of Formula Ia or Formula Ib, as one intramuscular injection of about 500 mg/mL, once every four months. In some embodiments, the method comprises intramuscularly administering about 1000 mg of the free acid form of the compound of Formula Ia or Formula Ib, as one intramuscular injection of about 500 mg/mL, once every four months.

In some embodiments, the method comprises intramuscularly administering about 1000 mg of the compound of Formula Ia or Formula Ib, as one intramuscular injection of 2 mL, at a concentration of about 500 mg/mL, once every four months. In some embodiments, the method comprises intramuscularly administering about 1000 mg of a sodium salt of the compound of Formula Ia or Formula Ib, as one intramuscular injection of 2 mL, at a concentration of about 500 mg/mL, once every four months. In some embodiments, the method comprises intramuscularly administering about 1000 mg of the free acid form of the compound of Formula Ia or Formula Ib, as one intramuscular injection of 2 mL, at a concentration of about 500 mg/mL, once every four months.

In some embodiments of the methods provided herein, a composition comprising:
about 30% to about 45% of the free acid form of the compound of Formula Ia;
about 40% to about 50% of PEG 300;
about 7% to about 15% of ethanol; and
about 5% to about 10% of water;
is administered intramuscularly to a patient in need thereof once every four months. In some embodiments, the above composition consists of the specified ingredients. In some embodiments, the above composition consists essentially of the specified ingredients.

In some embodiments of the methods provided herein, a composition comprising:
about 41% to about 43% of the free acid form of the compound of Formula Ia;
about 37% to about 43% of PEG 300;
about 9% to about 11% of ethanol; and
about 6% to about 8% of water;
is administered intramuscularly to a patient in need thereof once every four months. In some embodiments, the above composition consists of the specified ingredients. In some embodiments, the above composition consists essentially of the specified ingredients.

In some embodiments of the methods provided herein, a composition comprising:
about 42% of the free acid form of the compound of Formula Ia;
about 37% to about 43% of PEG 300;
about 9% to about 11% of ethanol; and
about 6% to about 8% of water;
is administered intramuscularly to a patient in need thereof once every four months. In some embodiments, the above composition consists of the specified ingredients. In some embodiments, the above composition consists essentially of the specified ingredients.

In some embodiments of the methods provided herein, a composition comprising:
about 42.02% of the free acid form of the compound of Formula Ia;
about 37% to about 43% of PEG 300;
about 9% to about 11% of ethanol; and
about 6% to about 8% of water;
is administered intramuscularly to a patient in need thereof once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every four months. In some embodiments, the above composition consists of the specified ingredients. In some embodiments, the above composition consists essentially of the specified ingredients.

In some embodiments of the methods provided herein, a composition comprising:
about 30% to about 45% of the free acid form of the compound of Formula Ia;
about 40% to about 50% of PEG 300;
about 7% to about 15% of ethanol; and
about 5% to about 10% of water;
is administered intramuscularly to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments of the methods provided herein, a composition comprising:
about 41% to about 43% of the free acid form of the compound of Formula Ia;
about 37% to about 43% of PEG 300;
about 9% to about 11% of ethanol; and
about 6% to about 8% of water;
is administered intramuscularly to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments of the methods provided herein, a composition comprising:
about 42% of the free acid form of the compound of Formula Ia;
about 37% to about 43% of PEG 300;
about 9% to about 11% of ethanol; and
about 6% to about 8% of water;
is administered intramuscularly to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments of the methods provided herein, a composition comprising:
about 42.02% of the free acid form of the compound of Formula Ia;
about 37% to about 43% of PEG 300;
about 9% to about 11% of ethanol; and
about 6% to about 8% of water;
is administered intramuscularly to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise preventing a human immunodeficiency virus (HIV) infection (for example, by PrEP and/or PEP), wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 30% to about 45% of the free acid form of the compound of Formula Ia;
about 40% to about 50% of PEG 300;
about 7% to about 15% of ethanol; and
about 5% to about 10% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 41% to about 43% of the free acid form of the compound of Formula Ia;
about 37% to about 43% of PEG 300;
about 9% to about 11% of ethanol; and
about 6% to about 8% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42% of the free acid form of the compound of Formula Ia;
about 37% to about 43% of PEG 300;
about 9% to about 11% of ethanol; and
about 6% to about 8% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments the methods provided herein comprise treating a human immunodeficiency virus (HIV) infection, wherein the method comprises intramuscularly administering a composition comprising:
about 42.02% of the free acid form of the compound of Formula Ia;
about 37% to about 43% of PEG 300;
about 9% to about 11% of ethanol; and
about 6% to about 8% of water;
to a patient in need thereof once every four months, wherein the method comprises administering about 1000 mg of the free acid form of the compound of Formula Ia, as one intramuscular injection of 2 mL, at a concentration of about 500 mg/mL, once every four months. In some embodiments, the above composition consists of the specified ingredients. In some embodiments, the above composition consists essentially of the specified ingredients.

In some embodiments, the methods provided herein comprise administering one intramuscular injection of Composition 3, once every four months.

In some embodiments, the methods provided herein comprise administering one intramuscular injection of about 2 mL of Composition 3, once every four months.

In some embodiments, the methods provided herein comprise administering about 1000 mg of the free acid form of the compound of Formula Ia or Formula Ib, as one intramuscular injection of about 2 mL of Composition 3, once every four months.

In some embodiments, the methods provided herein comprise administering about 1000 mg of the free acid form of the compound of Formula Ia, as one intramuscular injection of about 2 mL of Composition 3, once every four months.

In some embodiments, the methods provided herein comprise administering about 1000 mg of the free acid form of the compound of Formula Ib, as one intramuscular injection of about 2 mL of Composition 3, once every four months.

In some embodiments, the pharmaceutical compositions provided herein are administered as a monotherapy.

In some embodiments, the methods provided herein further comprise administering one or more initiation (i.e., loading) doses of the compound of Formula Ia or Formula Ib, or a pharmaceutically acceptable salt thereof.

In some embodiments, the one or more initiation dosages are administered to the patient prior to administration of a composition provided herein (e.g., a composition comprising a compound of Formula Ia or Ib, or a pharmaceutically acceptable salt or free acid form thereof).

In some embodiments, the one or more initiation dosages are administered to the patient prior to intramuscular administration of a composition provided herein (e.g., an intramuscular composition comprising a compound of Formula Ia or Ib, or a pharmaceutically acceptable salt or free acid form thereof).

In some embodiments, the one or more initiation dosages are administered to the patient prior to subcutaneous administration of a composition provided herein (e.g., a subcutaneous composition comprising a compound of Formula Ia or Ib, or a pharmaceutically acceptable salt or free acid form thereof).

In some embodiments, the methods provided herein further comprise administering one or more initiation (i.e., loading) doses of the compound of Formula Ia or Formula Ib, or a pharmaceutically acceptable salt thereof, for a first period of time.

In some embodiments, the first period of time is about one day to about 10 days. In some embodiments, the first period of time is one day. In some embodiments, the first period of time is two days.

In some embodiments, the initiation dosage provided herein comprises one or more subcutaneous administrations of the compound of Formula Ia or Ib, or a pharmaceutically acceptable salt thereof.

In some embodiments, the initiation dosage provided herein comprises one or more intramuscular administrations of the compound of Formula Ia or Ib, or a pharmaceutically acceptable salt thereof.

In some embodiments, the initiation dosage provided herein comprises one or more oral administrations of the compound of Formula Ia or Formula Ib, or a pharmaceutically acceptable salt thereof.

In some embodiments, the initiation dosage provided herein comprises one or more subcutaneous administrations of the compound of Formula Ia or Ib, or a pharmaceutically acceptable salt thereof; and
one or more oral administrations of the compound of Formula Ia or Ib, or a pharmaceutically acceptable salt thereof.

In some embodiments, the initiation dosage provided herein comprises one or more
intramuscular administrations of the compound of Formula Ia or Ib, or a pharmaceutically acceptable salt thereof; and
one or more oral administrations of the compound of Formula Ia or Ib, or a pharmaceutically acceptable salt thereof.

In some embodiments, the initiation dosage comprises:
intramuscularly administering to the patient about 2900 mg to about 3100 mg of the compound of Formula Ia or Ib, or a pharmaceutically acceptable salt thereof, on the first day;
orally administering to the patient about 500 mg to about 700 mg of the compound of Formula Ia or Ib, or a pharmaceutically acceptable salt thereof, on the first day; and
orally administering to the patient about 500 mg to about 700 mg of the compound of Formula Ia or Ib, or a pharmaceutically acceptable salt thereof, on the second day.

In some embodiments, the initiation dosage comprises:
intramuscularly administering to the patient the compound of Formula Ia or Ib, or a pharmaceutically acceptable salt thereof, at a concentration of about 500 mg/mL, on the first day;
orally administering to the patient about 500 mg to about 700 mg of the compound of Formula Ia or Ib, or a pharmaceutically acceptable salt thereof, on the first day; and
orally administering to the patient about 500 mg to about 700 mg of the compound of Formula Ia or Ib, or a pharmaceutically acceptable salt thereof, on the second day.

In some embodiments, the intramuscular administration of the initiation dosage comprises administering to the patient two intramuscular injections of the compound of Formula Ia, or a pharmaceutically acceptable salt thereof, each at a concentration of about 500 mg/mL. In some embodiments, the intramuscular administration of the initiation dosage comprises administering to the patient two intramuscular injections of the compound of Formula Ia, or a pharmaceutically acceptable salt thereof, each at a concentration of about 500 mg/mL, on the first day. In some embodiments, the intramuscular administration of the initiation dosage comprises administering to the patient two 3 mL intramuscular injections of the compound of Formula Ia, or a pharmaceutically acceptable salt thereof, each at a concentration of about 500 mg/mL, on the first day.

In some embodiments, the oral administrations of the initiation dosage are administered as one or two tablets (e.g., one or two tablets on the first day; one or two tablets on the second day; 1 or 2 tablets on the first day and 1 or 2 tablets on the second day; and the like). In some embodiments, the oral administrations of the initiation dosage are administered as two tablets on the first day, and two tablets on the second day.

In some embodiments, the oral administrations of the initiation dosage comprise administering to the patient two tablets on the first day and two tablets on the second day, wherein each tablet comprises about 200 to about 400 mg of the compound of Formula Ia or Ib, or a pharmaceutically acceptable salt thereof. In some embodiments, the oral administrations of the initiation dosage comprise administering to the patient two tablets on the first day and two tablets on the second day, wherein each tablet comprises about 300 mg of the compound of Formula Ia or Ib, or a pharmaceutically acceptable salt thereof.

In some embodiments, the oral administrations of the initiation dosage comprise administering to the patient two tablets on the first day and two tablets on the second day, wherein each tablet comprises about 200 to about 400 mg of the compound of Formula Ia, or a pharmaceutically acceptable salt thereof. In some embodiments, the oral administrations of the initiation dosage comprise administering to the patient two tablets on the first day and two tablets on the second day, wherein each tablet comprises about 300 mg of the compound of Formula Ia, or a pharmaceutically acceptable salt thereof.

In some embodiments, the oral administrations of the initiation dosage comprise administering to the patient two tablets on the first day and two tablets on the second day, wherein each tablet comprises about 200 to about 400 mg of the compound of Formula Ib, or a pharmaceutically acceptable salt thereof. In some embodiments, the oral administrations of the initiation dosage comprise administering to the patient two tablets on the first day and two tablets on the second day, wherein each tablet comprises about 300 mg of the compound of Formula Ib, or a pharmaceutically acceptable salt thereof.

In some embodiments, the initiation dosage comprises:
intramuscularly administering to the patient about 3000 mg of the compound of Formula Ia or Ib, or a pharmaceutically acceptable salt thereof, on the first day;
orally administering to the patient about 600 mg of the compound of Formula Ia or Ib, or a pharmaceutically acceptable salt thereof, on the first day; and
orally administering to the patient about 600 mg of the compound of Formula Ia or Ib, or a pharmaceutically acceptable salt thereof, on the second day.

In some embodiments, the initiation dosage comprises:
intramuscularly administering to the patient about 3000 mg of the compound of Formula Ia, or a pharmaceutically acceptable salt thereof, on the first day;
orally administering to the patient about 600 mg of the compound of Formula Ia, or a pharmaceutically acceptable salt thereof, on the first day; and
orally administering to the patient about 600 mg of the compound of Formula Ia, or a pharmaceutically acceptable salt thereof, on the second day.

In some embodiments, the initiation dosage comprises:
intramuscularly administering to the patient about 3000 mg of the compound of Formula Ib, or a pharmaceutically acceptable salt thereof, on the first day;
orally administering to the patient about 600 mg of the compound of Formula Ib, or a pharmaceutically acceptable salt thereof, on the first day; and
orally administering to the patient about 600 mg of the compound of Formula Ib, or a pharmaceutically acceptable salt thereof, on the second day.

In some embodiments, the first period of time is two days and the initiation dosage comprises:
intramuscularly administering to the patient about 3000 mg of the compound of Formula Ia or Ib, or a pharmaceutically acceptable salt thereof, on the first day;
orally administering to the patient about 600 mg of the compound of Formula Ia or Ib, or a pharmaceutically acceptable salt thereof, on the first day; and
orally administering to the patient about 600 mg of the compound of Formula Ia or Ib, or a pharmaceutically acceptable salt thereof, on the second day.

In some embodiments, the first period of time is two days and the initiation dosage comprises:
intramuscularly administering to the patient about 3000 mg of the compound of Formula Ia, or a pharmaceutically acceptable salt thereof, on the first day;
orally administering to the patient about 600 mg of the compound of Formula Ia, or a pharmaceutically acceptable salt thereof, on the first day; and
orally administering to the patient about 600 mg of the compound of Formula Ia, or a pharmaceutically acceptable salt thereof, on the second day.

In some embodiments, the first period of time is two days and the initiation dosage comprises:
intramuscularly administering to the patient about 3000 mg of the compound of Formula Ib, or a pharmaceutically acceptable salt thereof, on the first day;
orally administering to the patient about 600 mg of the compound of Formula Ib, or a pharmaceutically acceptable salt thereof, on the first day; and
orally administering to the patient about 600 mg of the compound of Formula Ib, or a pharmaceutically acceptable salt thereof, on the second day.

In some embodiments, the initiation dosage comprises:
intramuscularly administering to the patient about 3000 mg of the compound of Formula Ia or Ib, or a pharmaceutically acceptable salt thereof, as two 3 mL intramuscular injections of the compound of Formula Ia or Ib, or a pharmaceutically acceptable salt thereof, each at a concentration of about 500 mg/mL, on the first day;
orally administering to the patient about 600 mg of the compound of Formula Ia or Ib, or a pharmaceutically acceptable salt thereof, on the first day; and
orally administering to the patient about 600 mg of the compound of Formula Ia or Ib, or a pharmaceutically acceptable salt thereof, on the second day.

In some embodiments, the initiation dosage comprises:
intramuscularly administering to the patient about 3000 mg of the compound of Formula Ia, or a pharmaceutically acceptable salt thereof, as two 3 mL intramuscular injections of the compound of Formula Ia, or a pharmaceutically acceptable salt thereof, each at a concentration of about 500 mg/mL, on the first day;
orally administering to the patient about 600 mg of the compound of Formula Ia, or a pharmaceutically acceptable salt thereof, on the first day; and
orally administering to the patient about 600 mg of the compound of Formula Ia, or a pharmaceutically acceptable salt thereof, on the second day.

In some embodiments, the initiation dosage comprises:
intramuscularly administering to the patient about 3000 mg of the compound of Formula Ib, or a pharmaceutically acceptable salt thereof, as two 3 mL intramuscular injections of the compound of Formula Ib, or a pharmaceutically acceptable salt thereof, each at a concentration of about 500 mg/mL, on the first day;
orally administering to the patient about 600 mg of the compound of Formula Ib, or a pharmaceutically acceptable salt thereof, on the first day; and
orally administering to the patient about 600 mg of the compound of Formula Ib, or a pharmaceutically acceptable salt thereof, on the second day.

In some embodiments, the first period of time is two days and the initiation dosage comprises:
intramuscularly administering to the patient about 3000 mg of the compound of Formula Ia or Ib, or a pharmaceutically acceptable salt thereof, as two 3 mL intramuscular injections of the compound of Formula Ia or Ib, or a pharmaceutically acceptable salt thereof, each at a concentration of about 500 mg/mL, on the first day;
orally administering to the patient about 600 mg of the compound of Formula Ia or Ib, or a pharmaceutically acceptable salt thereof, on the first day; and
orally administering to the patient about 600 mg of the compound of Formula Ia or Ib, or a pharmaceutically acceptable salt thereof, on the second day.

In some embodiments, the first period of time is two days and the initiation dosage comprises:
intramuscularly administering to the patient about 3000 mg of the compound of Formula Ia, or a pharmaceutically acceptable salt thereof, as two 3 mL intramuscular injections of the compound of Formula Ia, or a pharmaceutically acceptable salt thereof, each at a concentration of about 500 mg/mL, on the first day;
orally administering to the patient about 600 mg of the compound of Formula Ia, or a pharmaceutically acceptable salt thereof, on the first day; and
orally administering to the patient about 600 mg of the compound of Formula Ia, or a pharmaceutically acceptable salt thereof, on the second day.

In some embodiments, the first period of time is two days and the initiation dosage comprises:
intramuscularly administering to the patient about 3000 mg of the compound of Formula Ib, or a pharmaceutically acceptable salt thereof, as two 3 mL intramuscular injections of the compound of Formula Ib, or a pharmaceutically acceptable salt thereof, each at a concentration of about 500 mg/mL, on the first day;
orally administering to the patient about 600 mg of the compound of Formula Ib, or a pharmaceutically acceptable salt thereof, on the first day; and
orally administering to the patient about 600 mg of the compound of Formula Ib, or a pharmaceutically acceptable salt thereof, on the second day.

In some embodiments, the initiation dosage is administered orally as one or two tablets (e.g., one or two tablets on the first day; one or two tablets on the second day; one or two tablets on the first day and one or two tablets on the second day; and the like). In some embodiments, the oral administrations are administered as two tablets.

In some embodiments, the initiation dosage is administered orally as two tablets, each comprising about 200 to about 400 mg of the compound of Formula Ia or Ib, or a pharmaceutically acceptable salt thereof, on the first day and on the second day.

In some embodiments, the initiation dosage is administered orally as two tablets each comprising about 200 to about 400 mg of the compound of Formula Ia or Ib, or a pharmaceutically acceptable salt thereof, on the first day; and as two tablets each comprising about 200 to about 400 mg of the compound of Formula Ia or Ib, or a pharmaceutically acceptable salt thereof, on the second day.

In some embodiments, the initiation dosage is administered orally as two tablets, each comprising about 200 to about 400 mg of the compound of Formula Ia or Ib, or a pharmaceutically acceptable salt thereof, on the first day; and as two tablets, each comprising about 200 to about 400 mg of the compound of Formula Ia or Ib, or a pharmaceutically acceptable salt thereof, on the second day.

In some embodiments, the initiation dosage is administered orally as two tablets, each comprising about 300 mg of the compound of Formula Ia or Ib, or a pharmaceutically acceptable salt thereof, on the first day and on the second day.

In some embodiments, the initiation dosage is administered orally as two tablets, each comprising about 300 mg of the compound of Formula Ia or Ib, or a pharmaceutically acceptable salt thereof, on the first day and on the second day.

In some embodiments, the initiation dosage is administered orally as two tablets, each comprising about 300 mg of the compound of Formula Ia or Ib, or a pharmaceutically acceptable salt thereof, on the first day; and as two tablets, each comprising about 300 mg of the compound of Formula Ia or Ib, or a pharmaceutically acceptable salt thereof, on the second day.

In some embodiments, the intramuscular formulation of the initiation dosage is a solution comprising a compound of Formula Ia or Ib, or a pharmaceutically acceptable salt thereof, PEG 300, ethanol, and water. In some embodiments, the intramuscular formulation of the initiation dosage is a solution comprising a sodium salt of the compound of Formula Ia or Ib, PEG 300, ethanol, and water. In some embodiments, the intramuscular formulation of the initiation dosage is a solution comprising a sodium salt of the compound of Formula Ia, PEG 300, ethanol, and water. In some embodiments, the intramuscular formulation of the initiation dosage is a solution comprising a sodium salt of the compound of Formula Ib, PEG 300, ethanol, and water.

In some embodiments, the intramuscular formulation of the initiation dosage comprises:
about 30% to about 45% of the free acid form of the compound of Formula Ia;
about 37% to about 50% of the PEG 300;
about 5% to about 10% of the water; and
about 7% to about 15% of the ethanol.
   In some embodiments, the above formulation consists of the specified ingredients. In some embodiments, the above formulation consists essentially of the specified ingredients.

In some embodiments, the intramuscular formulation of the initiation dosage comprises:
about 30% to about 45% of the free acid form of the compound of Formula Ia;
about 40% to about 50% of the PEG 300;
about 5% to about 10% of the water; and
about 7% to about 15% of the ethanol.
   In some embodiments, the above formulation consists of the specified ingredients. In some embodiments, the above formulation consists essentially of the specified ingredients.

In some embodiments, the intramuscular formulation of the initiation dosage comprises:
about 41% to about 43% of the free acid form of the compound of Formula Ia;
about 37% to about 43% of the PEG 300;
about 6% to about 8% of the water; and
about 9% to about 11% of the ethanol.
   In some embodiments, the above formulation consists of the specified ingredients. In some embodiments, the above formulation consists essentially of the specified ingredients.

In some embodiments, the intramuscular formulation of the initiation dosage comprises:
about 42% of the free acid form of the compound of Formula Ia;
about 37% to about 43% of the PEG 300;
about 6% to about 8% of the water; and
about 9% to about 11% of the ethanol.
   In some embodiments, the above formulation consists of the specified ingredients. In some embodiments, the above formulation consists essentially of the specified ingredients.

In some embodiments, the intramuscular formulation of the initiation dosage comprises:
about 42.02% of the free acid form of the compound of Formula Ia;
about 37% to about 43% of the PEG 300;
about 6% to about 8% of the water; and
about 9% to about 11% of the ethanol.
   In some embodiments, the above formulation consists of the specified ingredients. In some embodiments, the above formulation consists essentially of the specified ingredients.

In some embodiments, the intramuscular formulation of the initiation dosage is Composition 3.

In some embodiments, the tablet for the initiation dosage provided herein is prepared from a spray-dried dispersion of a compound of Formula Ia or Ib, or a pharmaceutically acceptable salt thereof. In some embodiments, the amount of a compound of Formula Ia or Ib, or a pharmaceutically acceptable salt thereof, in the tablet is about 5 mg to about 500 mg. In some embodiments, the amount of a compound of Formula Ia or Ib, or a pharmaceutically acceptable salt thereof, in the tablet is about 25 mg to about 500 mg. In some embodiments, the amount of a compound of Formula Ia or Ib, or a pharmaceutically acceptable salt thereof, in the tablet is about 25 mg to about 400 mg. In some embodiments, the amount of a compound of Formula Ia or Ib, or a pharmaceutically acceptable salt thereof, in the tablet is about 25 mg to about 300 mg. In some embodiments, the amount of a compound of Formula Ia or Ib, or a pharmaceutically acceptable salt thereof, in the tablet is about 50 mg to about 500 mg. In some embodiments, the amount of a compound of Formula Ia or Ib, or a pharmaceutically acceptable salt thereof, in the tablet is about 75 mg to about 500 mg. In some embodiments, the amount of a compound of Formula Ia or Ib, or a pharmaceutically acceptable salt thereof, in the tablet is about 50 mg to about 400 mg. In some embodiments, the amount of a compound of Formula Ia or Ib, or a pharmaceutically acceptable salt thereof, in the tablet is about 50 mg to about 300 mg. In some embodiments, the amount of a compound of Formula Ia or Ib, or a pharmaceutically acceptable salt thereof, in the tablet is about 75 mg to about 400 mg. In some embodiments, the amount of a compound of Formula Ia or Ib, or a pharmaceutically acceptable salt thereof, in the tablet is about 75 mg to about 300 mg. In some embodiments, the amount of a compound of Formula Ia or Ib, or a pharmaceutically acceptable salt thereof, in the tablet is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, or about 500 mg. In some embodiments, the amount of a compound of Formula Ia or Ib, or a pharmaceutically acceptable salt thereof, in the tablet is about 5 mg. In some embodiments, the amount of a compound of Formula Ia or Ib, or a pharmaceutically acceptable salt thereof, in the tablet is about 10 mg. In some embodiments, the amount of a compound of Formula Ia or Ib, or a pharmaceutically acceptable salt thereof, in the tablet is about 20 mg. In some embodiments, the amount of a compound of Formula Ia or Ib, or a pharmaceutically acceptable salt thereof, in the tablet is about 25 mg. In some embodiments, the amount of a compound of Formula Ia or Ib, or a pharmaceutically acceptable salt thereof, in the tablet is about 30 mg. In some embodiments, the amount of a compound of Formula Ia or Ib, or a pharmaceutically acceptable salt thereof, in the tablet is about 40 mg. In some embodiments, the amount of a compound of Formula Ia or Ib, or a pharmaceutically acceptable salt thereof, in the tablet is about 50 mg. In some embodiments, the amount of a compound of Formula Ia or Ib, or a pharmaceutically acceptable salt thereof, in the tablet is about 75 mg. In some embodiments, the amount of a compound of Formula Ia or Ib, or a pharmaceutically acceptable salt thereof, in the tablet is about 100 mg. In some embodiments, the amount of a compound of Formula Ia or Ib, or a pharmaceutically acceptable salt thereof, in the tablet is about 125 mg. In some embodiments, the amount of a compound of Formula Ia or Ib, or a pharmaceutically acceptable salt thereof, in the tablet is about 150 mg. In some embodiments, the amount of a compound of Formula Ia or Ib, or a pharmaceutically acceptable salt thereof, in the tablet is about 175 mg. In some embodiments, the amount of a compound of Formula Ia or Ib, or a pharmaceutically acceptable salt thereof, in the tablet is about 200 mg. In some embodiments, the amount of a compound of Formula Ia or Ib, or a pharmaceutically acceptable salt thereof, in the tablet is about 225 mg. In some embodiments, the amount of a compound of Formula Ia or Ib, or a pharmaceutically acceptable salt thereof, in the tablet is about 250 mg. In some embodiments, the amount of a compound of Formula Ia or Ib, or a pharmaceutically acceptable salt thereof, in the tablet is about 275 mg. In some embodiments, the amount of a compound of Formula Ia or Ib, or a pharmaceutically acceptable salt thereof, in the tablet is about 300 mg.

In some embodiments, the tablet comprises a pharmaceutically acceptable salt of the compound of Formula Ia or Ib. In some embodiments, the tablet comprises a pharmaceutically acceptable salt of the compound of Formula Ia. In some embodiments, the tablet comprises a pharmaceutically acceptable salt of the compound of Formula Ib. In some embodiments, the tablet comprises a sodium salt of the compound of Formula Ia or Ib. In some embodiments, the tablet comprises a sodium salt of the compound of Formula Ia. In some embodiments, the tablet comprises a sodium salt of the compound of Formula Ib.

In some embodiments, the tablet comprises the free acid form of the compound of Formula Ia or Ib. In some embodiments, the tablet comprises the free acid form of the compound of Formula Ia. In some embodiments, the tablet comprises the free acid form of the compound of Formula Ib. In some embodiments, the amount of the free acid of the compound of Formula Ia or Ib, in the tablet is about 300 mg. In some embodiments, the amount of the free acid of the compound of Formula Ib, in the tablet is about 300 mg.

In some embodiments, the amount of a compound of Formula Ia or Ib, or a pharmaceutically acceptable salt thereof, in the tablet is about 325 mg. In some embodiments, the amount of a compound of Formula Ia or Ib, or a pharmaceutically acceptable salt thereof, in the tablet is about 350 mg. In some embodiments, the amount of a compound of Formula Ia or Ib, or a pharmaceutically acceptable salt thereof, in the tablet is about 375 mg. In some embodiments, the amount of a compound of Formula Ia or Ib, or a pharmaceutically acceptable salt thereof, in the tablet is about 400 mg. In some embodiments, the amount of a compound of Formula Ia or Ib, or a pharmaceutically acceptable salt thereof, in the tablet is about 425 mg. In some embodiments, the amount of a compound of Formula Ia or Ib, or a pharmaceutically acceptable salt thereof, in the tablet is about 450 mg. In some embodiments, the amount of a compound of Formula Ia or Ib, or a pharmaceutically acceptable salt thereof, in the tablet is about 475 mg. In some embodiments, the amount of a compound of Formula Ia or Ib, or a pharmaceutically acceptable salt thereof, in the tablet is about 500 mg.

In some embodiments, the tablet herein comprises a compound of Formula Ia or Ib, or a pharmaceutically acceptable salt thereof, and one or more pharmaceutically acceptable excipients. In some embodiments, the tablet comprises a compound of Formula Ia or a pharmaceutically acceptable salt thereof, and one or more pharmaceutically acceptable excipients. In some embodiments, the tablet comprises a compound of Formula Ia and one or more pharmaceutically acceptable excipients. In some embodiments, the tablet comprises a sodium salt of the compound of Formula Ia and one or more pharmaceutically acceptable excipients. In some embodiments, the tablet comprises a compound of Formula Ib or a pharmaceutically acceptable salt thereof, and one or more pharmaceutically acceptable excipients. In some embodiments, the tablet comprises a compound of Formula Ib and one or more pharmaceutically acceptable excipients. In some embodiments, the tablet comprises a sodium salt of the compound of Formula Ib and one or more pharmaceutically acceptable excipients.

In some embodiments, the tablets disclosed herein comprise a compound of Formula Ia or Ib, or a pharmaceutically acceptable salt thereof, and one or more pharmaceutically acceptable excipients selected from the group consisting of a matrix former, a surfactant, a filler, a disintegrant, and a lubricant. In some embodiments, the tablet comprises about 1 w/w % to about 10 w/w % of a matrix former. In some embodiments, the matrix former comprises copovidone. In some embodiments, the tablet comprises about 0.01 w/w % to about 10 w/w % of a surfactant. In some embodiments, the surfactant comprises poloxamer 407. In some embodiments, the tablet comprises about 25-85 w/w % of one or more fillers. In some embodiments, the one or more fillers comprises microcrystalline cellulose and/or mannitol. In some embodiments, the tablet comprises about 1 w/w % to about 30 w/w % of a disintegrant. In some embodiments, the disintegrant comprises croscarmellose sodium. In some embodiments, the tablet comprises about 0.01 w/w % to about 10 w/w % of a lubricant. In some embodiments, the lubricant comprises magnesium stearate.

The tablets disclosed herein may be uncoated or coated (in which case they include an outer film coat). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water-soluble material included in the film coating of the tablets may include a single polymer material, it may also be formed using a mixture of more than one polymer. The coating may be white or colored. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional colouring such as iron oxide or indigo carmine or iron oxide yellow or FD&C yellow #6). The amount of coating will generally be between about 1-8% of the uncoated tablet's weight.

In some embodiments, the tablet disclosed herein comprises a compound of Formula Ia or Ib or a pharmaceutically acceptable salt thereof, and one or more pharmaceutically acceptable excipients. In some embodiments, the one or more pharmaceutically acceptable excipients is selected from the group consisting of copovidone, poloxamer 407, microcrystalline cellulose, mannitol, croscarmellose sodium, and magnesium stearate. In some embodiments, the one or more pharmaceutically acceptable excipient comprises copovidone. In some embodiments, the one or more pharmaceutically acceptable excipient comprises poloxamer 407. In some embodiments, the one or more pharmaceutically acceptable excipient comprises microcrystalline cellulose. In some embodiments, the one or more pharmaceutically acceptable excipient comprises mannitol. In some embodiments, the one or more pharmaceutically acceptable excipient comprises croscarmellose sodium. In some embodiments, the one or more pharmaceutically acceptable excipient comprises magnesium stearate. In some embodiments, the one or more pharmaceutically acceptable excipient comprises copovidone and poloxamer 407. In some embodiments, the one or more pharmaceutically acceptable excipient comprises copovidone, poloxamer 407, and microcrystalline cellulose. In some embodiments, the one or more pharmaceutically acceptable excipient comprises copovidone, poloxamer 407, microcrystalline cellulose, and mannitol. In some embodiments, the one or more pharmaceutically acceptable excipient comprises copovidone, poloxamer 407, microcrystalline cellulose, mannitol, and croscarmellose sodium. In some embodiments, the one or more pharmaceutically acceptable excipient comprises copovidone, poloxamer 407, microcrystalline cellulose, mannitol, croscarmellose sodium, and magnesium stearate.

In some embodiments, the tablet disclosed herein comprises a compound of Formula Ia or Ib or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ia, or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ib, or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ia, copovidone, poloxamer 407, microcrystalline cellulose, mannitol, croscarmellose sodium, and magnesium stearate. In some embodiments, the tablet comprises a sodium salt of the compound of Formula Ia, copovidone, poloxamer 407, microcrystalline cellulose, mannitol, croscarmellose sodium, and magnesium stearate. In some embodiments, the tablet comprises a compound of Formula Ib, copovidone, poloxamer 407, microcrystalline cellulose, mannitol, croscarmellose sodium, and magnesium stearate. In some embodiments, the tablet comprises a sodium salt of the compound of Formula Ib, copovidone, poloxamer 407, microcrystalline cellulose, mannitol, croscarmellose sodium, and magnesium stearate.

In some embodiments, the tablet disclosed herein comprises a sodium salt of the compound of Formula Ia or Ib, copovidone, poloxamer 407, microcrystalline cellulose, mannitol, croscarmellose sodium, and magnesium stearate. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 mg to about 500 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 500 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 400 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 50 mg to about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 10 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 30 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 40 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 50 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 75 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 100 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 125 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 150 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 175 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 200 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 225 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 250 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 275 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 325 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 350 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 375 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 400 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 425 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 450 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 475 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 500 mg.

In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 w/w % to about 45 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 10 w/w % to about 40 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 15 w/w % to about 35 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 15 w/w % to about 25 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20.46 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20.5 w/w %.

In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 1 w/w % to about 10 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 2 w/w % to about 10 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 3 w/w % to about 8 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 3 w/w % to about 6 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 4.88 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 4.9 w/w %.

In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.01 w/w % to about 10 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.05 w/w % to about 8 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 4 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 3.0 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 1.3 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 1.33 w/w %.

In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 5 w/w % to about 45 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 10 w/w % to about 40 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 15 w/w % to about 35 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 18 w/w % to about 30 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 21.28 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 21.3 w/w %.

In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 15 w/w % to about 70 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 20 w/w % to about 60 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 30 w/w % to about 55 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 40 w/w % to about 50 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 42.55 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 42.6 w/w %.

In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 1 w/w % to about 30 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 1 w/w % to about 20 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 4 w/w % to about 16 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 6 w/w % to about 10 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 8.0 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 8.00 w/w %.

In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.01 w/w % to about 10 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.05 w/w % to about 8 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 4 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.0 w/w % to about 3.0 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.5 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.50 w/w %.

In some embodiments, the tablet disclosed herein comprises about 5 w/w % to about 45 w/w % of a sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, about 0.01 w/w % to about 10 w/w % of magnesium stearate, and one or more pharmaceutically acceptable excipients. In some embodiments, the tablet disclosed herein comprises about 5 w/w % to about 45 w/w % of a sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, and about 0.01 w/w % to about 10 w/w % of magnesium stearate. In some embodiments, the tablet comprises about 10 w/w % to about 40 w/w % of a sodium salt of the compound of Formula Ia or Ib, about 2 w/w % to about 10 w/w % of copovidone, about 0.05 w/w % to about 8 w/w % of poloxamer 407, about 10 w/w % to about 40 w/w % of microcrystalline cellulose, about 20 w/w % to about 60 w/w % of mannitol, about 1 w/w % to about 20 w/w % of croscarmellose sodium, and about 0.05 w/w % to about 8 w/w % of magnesium stearate. In some embodiments, the tablet comprises about 15 w/w % to about 35 w/w % of a sodium salt of the compound of Formula Ia or Ib, about 3 w/w % to about 8 w/w % of copovidone, about 0.5 w/w % to about 4 w/w % of poloxamer 407, about 15 w/w % to about 35 w/w % of microcrystalline cellulose, about 30 w/w % to about 55 w/w % of mannitol, about 4 w/w % to about 16 w/w % of croscarmellose sodium, and about 0.5 w/w % to about 4 w/w % of magnesium stearate. In some embodiments, the tablet comprises about 15 w/w % to about 25 w/w % of a sodium salt of the compound of Formula Ia or Ib, about 3 w/w % to about 6 w/w % of copovidone, about 0.5 w/w % to about 3.0 w/w % of poloxamer 407, about 18 w/w % to about 30 w/w % of microcrystalline cellulose, about 40 w/w % to about 50 w/w % of mannitol, about 6 w/w % to about 10 w/w % of croscarmellose sodium, and about 1.0 w/w % to about 3.0 w/w % of magnesium stearate.

In some embodiments, the tablet comprises about 20.5 w/w % of a sodium salt of the compound of Formula Ia or Ib, about 4.9 w/w % of copovidone, about 1.3 w/w % of poloxamer 407, about 21.3 w/w % of microcrystalline cellulose, about 42.6 w/w % of mannitol, about 8.0 w/w % of croscarmellose sodium, and about 1.5 w/w % of magnesium stearate. In some embodiments, the tablet comprises about 20.46 w/w % of a sodium salt of the compound of Formula Ia or Ib, about 4.88 w/w % of copovidone, about 1.33 w/w % of poloxamer 407, about 21.28 w/w % of microcrystalline cellulose, about 42.55 w/w % of mannitol, about 8.00 w/w % of croscarmellose sodium, and about 1.50 w/w % of magnesium stearate. In some embodiments, the tablet comprises about 20.5 w/w % of a sodium salt of the compound of Formula Ib, about 4.9 w/w % of copovidone, about 1.3 w/w % of poloxamer 407, about 21.3 w/w % of microcrystalline cellulose, about 42.6 w/w % of mannitol, about 8.0 w/w % of croscarmellose sodium, and about 1.5 w/w % of magnesium stearate. In some embodiments, the tablet comprises about 20.46 w/w % of a sodium salt of the compound of Formula Ib, about 4.88 w/w % of copovidone, about 1.33 w/w % of poloxamer 407, about 21.28 w/w % of microcrystalline cellulose, about 42.55 w/w % of mannitol, about 8.00 w/w % of croscarmellose sodium, and about 1.50 w/w % of magnesium stearate.

In some embodiments, the tablet disclosed herein further comprises an outer film coat. In some embodiments, the tablet comprising a sodium salt of the compound of Formula Ia or Ib, copovidone, poloxamer 407, microcrystalline cellulose, mannitol, croscarmellose sodium, and magnesium stearate further comprises an outer film coat. In some embodiments, the outer film coat provides from about 1% to about 8% weight gain based on the uncoated tablet. In some embodiments, the outer film coat provides from about 2% to about 6% weight gain based on the uncoated tablet. In some embodiments, the outer film coat provides from about 2% to about 4% weight gain based on the uncoated tablet. In some embodiments, the outer film coat provides from about 4% to about 6% weight gain based on the uncoated tablet. In some embodiments, the outer film coat provides about 1%, about 2%, about 3%, about 4%, about 5%, about 6%, about 7%, or about 8% weight gain based on the uncoated tablet. In some embodiments, the outer film coat provides about 1% weight gain based on the uncoated tablet. In some embodiments, the outer film coat provides about 2% weight gain based on the uncoated tablet. In some embodiments, the outer film coat provides about 3% weight gain based on the uncoated tablet. In some embodiments, the outer film coat provides about 4% weight gain based on the uncoated tablet. In some embodiments, the outer film coat provides about 5% weight gain based on the uncoated tablet. In some embodiments, the outer film coat provides about 6% weight gain based on the uncoated tablet. In some embodiments, the outer film coat provides about 7% weight gain based on the uncoated tablet. In some embodiments, the outer film coat provides about 8% weight gain based on the uncoated tablet. In some embodiments, the outer film coat comprises Opadry® II. In some embodiments, the outer film coat comprises Opadry® II White. In some embodiments, the outer film coat comprises Opadry® II White 85F18422.

In some embodiments, the outer film coat comprises Opadry® II Green. In some embodiments, the outer film coat comprises Opadry® II Green 85F110187. In some embodiments, the outer film coat comprises Opadry® II Green 85F110186.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 3% weight gain from Opadry® II White 85F18422, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 3.0% weight gain from Opadry® II White 85F18422, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 3% weight gain from Opadry® II White 85F18422, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 3.0% weight gain from Opadry® II White 85F18422, wherein the weight gain is based on the uncoated tablet.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110187, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110187, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110187, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110187, wherein the weight gain is based on the uncoated tablet.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110186, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110186, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110186, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110186, wherein the weight gain is based on the uncoated tablet.

### EXAMPLES

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. Evaluation of Safety, Tolerability, and Pharmacokinetics of Single Dose Subcutaneous (SC) and Intramuscular (IM) Lenacapavir (LEN)

The following study has been designed in four parts (Parts A-D) as described below. The primary objectives of this study were to evaluate the safety and tolerability of single doses of subcutaneous (SC) and intramuscular (IM) administration of LEN; and to characterize the pharmacokinetics (PK) of single doses of SC and IM administration of LEN.

### Part A

Part A of this study entails SC administration of solution formulations of LEN to participants. Data suggest altering the concentration of LEN within the formulation and addition or removal of a poloxamer excipient impacts the PK profile of LEN. The objective of Part A of the study is to evaluate the safety, tolerability, and PK of LEN 400 mg/mL and 500 mg/mL LEN sodium salt (NaS) SC injection formulations with a range of poloxamer concentrations (up to 7.6% for 400 mg/mL and 500 mg/mL formulations). Dose selection for SC LEN NaS were supported by PK and safety data from Phase 1 studies in healthy participants and Phase 1b proof-of-concept study in treatment-naive and treatment experienced but capsid inhibitor-naive people with HIV (PWH), as well as nonclinical safety data.

### Part B

Part B of this study entails SC or IM administration of solution formulations of LEN to participants. The objective of Part B of the study is to evaluate the safety, tolerability, and PK of LEN following up to 500 mg/mL NaS or free acid (FA), SC or IM injection formulations with a range of poloxamer concentrations (up to 7.6%).

### Part C

Part C of this study entails SC or IM administration of formulations of LEN injection NaS or FA with optional oral loading, which have been dosed in Part A and Part B of this study. The objective of Part C of the study is to evaluate the safety, tolerability, and PK of LEN up to 500 mg/mL NaS or FA SC or IM injection formulations with a range of poloxamer concentrations (up to 7.6% for 400 mg/mL and 500 mg/mL NaS or FA formulations).

### Part D

Part D of this study entails SC or IM administration of LEN injection FA formulations with optional oral loading to participants. The objective of Part D of the study is to evaluate the safety, tolerability, and PK of LEN injection, 300 to 500 mg/mL FA SC or IM injection formulations with a range of poloxamer concentrations (up to 7.6% for 300 to 500 mg/mL FA formulations) and ethanol. Dose selection for SC or IM LEN NaS or FA in Parts B-D of this study is supported by PK and safety data from the Phase 1 studies in healthy participants and Phase 1b proof-of-concept study in treatment naive and treatment experienced but capsid inhibitor-naive PWH, as well as nonclinical safety data.

### Study Design

This protocol describes an open-label, multicenter, multiple-cohort Phase 1 study to evaluate the safety and tolerability of single doses of SC and IM LEN in healthy participants. Study drug was administered in the morning, without regard to food, with participants receiving either a single SC dose of LEN or single IM dose of LEN on Day 1.

### Inclusion Criteria

Participants must have met all of the following inclusion criteria to be eligible for participation in this study:
1. Have the ability to understand and sign a written informed consent form (ICF) obtained prior to initiation of study procedures.
2. Be aged 18 through 55 years, inclusive at screening.
3. Be a nonsmoker. The use of nicotine-containing products must be discontinued 42 days prior to the administration of study drug.
4. Have a calculated body mass index not greater than 35.0 kg/m² at screening.
5. Have a CL_{cr} at least 90 mL/min using the Cockcroft-Gault method (see e.g., Cockcroft & Gault, Nephron, 1976; 16:31-41) based on serum creatinine and actual body weight, as measured at screening:

$$Male : \frac{( 140 - Age [ years ] ) \times ( Weight [ kg ] )}{72 \times ( Serum Creatinine [ mg / dL ] )} = {CL}_{cr} ( mL / min )$$

$$Female : \frac{( 140 - Age [ years ] ) \times ( Weight [ kg ] )}{72 \times ( Serum Creatinine [ mg / dL ] )} \times 0.85 = {CL}_{cr} ( mL / min )$$

6. Participants who engage in heterosexual intercourse must agree to use protocol-specified method(s) of contraception.
7. Participants have not donated blood within 56 days of study entry or plasma within 7 days of study entry and must refrain from blood donation from clinic admission, throughout the study confinement period, and continuing for at least 449 days following the administration of study drug.
8. Screening laboratory evaluations and 12-lead ECG evaluations must be without clinically significant abnormalities as assessed by the investigator.
9. Have liver biometric tests of alanine aminotransferase (ALT), aspartate aminotransferase (AST), alkaline phosphatase, and total bilirubin below or equal to the upper limit of normal at screening.
10. Must be willing and able to comply with all study requirements.
11. Must, in the opinion of the investigator, be in good health based upon medical history and physical examination, including vital signs.

### Exclusion Criteria

Participants meeting any of the following exclusion criteria were not enrolled in this study:
1) Positive serum or urine pregnancy test.
2) Breastfeeding female.
3) Is currently participating in or has participated in an interventional clinical study with an investigational medicinal product within 30 days prior to study dosing on Day 1 through the duration of the study.
4) Has a tattoo or other dermatological condition overlying the injection site which in the opinion of the investigator, may interfere with interpretation of injection site reactions (ISRs).
5) Have current alcohol or substance abuse judged by the investigator to potentially interfere with participant compliance or safety, or a positive drug or alcohol test at screening or baseline.
6) Assessed by the investigator as being at risk for HIV infection in the past 6 months. This may include, but not be limited to, 1 or more of the following risk factors: (a) unprotected vaginal or anal sex with a person with HIV, (b) sex work for money or drugs, (c) sexually transmitted infection, (d) injection of nonprescribed drugs for recreational use, (e) post- or PrEP.
7) Have a positive test result for HIV at screening (antigen (Ag)/antibody (Ab) test) or Day −1 (rapid test).
8) Have a positive test result for hepatitis B surface antigen (HBsAg), or hepatitis C virus (HCV) Ab at screening.
9) Have poor venous access that limits phlebotomy.
10) Have taken any prescription medications or over-the-counter medications, including herbal products, within 28 days prior to start of study drug dosing, with the exception of vitamins and/or acetaminophen and/or ibuprofen and/or hormonal contraceptive medications.
11) Have been treated with systemic steroids, immunosuppressant therapies, or chemotherapeutic agents within 3 months prior to screening or is expected to receive these agents during the study (eg, corticosteroids, immunoglobulins, and other immune- or cytokine-based therapies).
12) Have a history of any of the following:
   a. Significant serious skin disease, such as but not limited to rash, food allergy, eczema, psoriasis, or urticaria.
   b. Significant drug sensitivity or drug allergy (such as anaphylaxis or hepatoxicity).
   c. Known hypersensitivity to the study drug, its metabolites, or to formulation excipients.
   d. Significant cardiac disease (including history of myocardial infarction based on ECG and/or clinical history, any history of ventricular tachycardia, congestive heart failure, or dilated cardiomyopathy with left ventricular ejection fraction <40%), a family history of long QT interval syndrome, or unexplained death in an otherwise healthy individual between the ages of 1 and 30 years.
   e. Syncope, palpitations, or unexplained dizziness.
   f. Implanted defibrillator or pacemaker.
   g. Liver disease, including Gilbert syndrome.
   h. Severe peptic ulcer disease, gastroesophageal reflux disease, or other gastric acid hypersecretory conditions requiring prolonged (>6 months) medical treatment.
13) Have any serious or active medical or psychiatric illness (including depression) that, in the opinion of the investigator, would interfere with participant treatment, assessment, or compliance with the protocol. This would include renal, cardiac, hematological, hepatic, pulmonary (including chronic asthma), endocrine (including diabetes), central nervous, gastrointestinal (including an ulcer), vascular, metabolic (thyroid disorders, adrenal disease), immunodeficiency disorders, active infection, or malignancy that are clinically significant or requiring treatment.

### LEN Formulations

Exemplary IM and SC compositions administered during this study are provided in Table 1.

TABLE 1
Exemplary IM and SC Compositions for 12-Month Dosing

| Composition # | 1 | 2 | 3 | 4 | 5 | 6 | 7 |
|---|---|---|---|---|---|---|---|
| Mode of | SC | IM | IM | IM | SC | SC | SC |
| Administration | | | | | | | |
| LEN sodium | 34.50% | 41.85% | — | — | 33.61% | — | — |
| LEN free acid | — | — | 42.02% | 41.08% | — | 34.19% | 33.78% |
| LEN | 400 | 500 | 500 | 500 | 400 | 400 | 400 |
| concentration | | | | | | | |
| (mg/mL) | | | | | | | |
| PEG 300 | 41.24% | 36.22% | 40.78% | 45.83% | 41.09% | 47.44 | 52.03% |
| Poloxamer | 0.00% | 0.00% | 0.00% | 0.00% | 6.12% | 0.00% | 0.00% |
| Water for | 19.26% | 16.93% | 7.20% | 8.09% | 19.18% | 8.37% | 9.18% |
| Injection | | | | | | | |
| Ethanol | 5.00% | 5.00% | 10.00% | 5.00% | 0.00% | 10.00% | 5.00% |
| Total (%) | 100.00% | 100.00% | 100.00% | 100.00% | 100.00% | 100.00% | 100% |
| Density | 1.184 | 1.222 | 1.190 | 1.217 | 1.222 | 1.17 | |
| (g/mL) | | | | | | | |
| Viscosity | 483 | 1820 | 565 | 1296 | | | |
| at 20 C. | | | | | | | |
| (cP) | | | | | | | |

Following completion of screening and admission assessments, eligible participants were enrolled on Day 1 to receive study drug. All SC treatments were administered in the abdomen, without regard to food, with SC dose volumes not exceeding 2 mL per injection. All IM treatments were administered in the gluteal, deltoid, or thigh, without regard to food, with IM dose volumes not exceeding 5 mL per injection in the gluteus or thigh, or 2 mL per injection in the deltoid. LEN may be administered as multiple SC injections or multiple IM injections at different anatomical sites, as appropriate.

### Pharmacokinetic (PK) Assessments

### Plasma Pharmacokinetic Collection

Intensive PK sampling occurs relative to the morning dose of LEN at the time points specified below.
Day 1:0 (predose; ≤5 minutes before dose) 2, 4, 8, and 12 hours postdose
Day 2:24 and 36 hours postdose

A single anytime PK sample is then collected on Days 3, 4, 6, 8, 10, 15, 22, 29, 36, 43, 57, 71, 85, 113, 141, 169, 197, 225, 253, 281, 309, 337, 351, 365, 379, 393, 421, 449, and at the early termination (ET) visit (if applicable).

### Injection Site Examination

Injection site examinations are completed 3 hours after injection on Day 1, daily on Days 2 through 15, at each outpatient visit based on reported signs and symptoms, and ET visit, if applicable. Photographs of ISRs may be taken.

### Clinical Laboratory Assessments

Blood and urine samples for safety evaluations are collected throughout the study for the following laboratory analyses:
Hematology: hematocrit, hemoglobin, platelet count, red blood cell count, white blood cell count with differential (absolute and percentage), including lymphocytes, monocytes, neutrophils, eosinophils, basophils, and mean corpuscular volume.
Chemistry (fasting): alkaline phosphatase, AST, ALT, total bilirubin, direct and indirect bilirubin, total cholesterol, high-density lipoprotein, low-density lipoprotein, triglycerides, total protein, albumin, lactic acid dehydrogenase, creatine phosphokinase, bicarbonate, blood urea nitrogen, calcium, chloride, creatinine and CLcr (see below), lipase, glucose, phosphorus, magnesium, potassium, sodium, and uric acid.
Coagulation panel (screening only): prothrombin time, partial thromboplastin time, and international normalized ratio.
Serum pregnancy test (women of childbearing potential only).
Follicle-stimulating hormone testing (screening only): women who are younger than 54 years old and have ceased menstruating for at least 12 months but do not have documentation of ovarian hormonal failure.
HBsAg and HCV testing (screening only).
HIV Ag/Ab combination testing.

### Adverse Events

An adverse event (AE) is any untoward medical occurrence in a clinical study participant administered a study drug that does not necessarily have a causal relationship with the treatment. An AE can, therefore, be any unfavorable and/or unintended sign, symptom, or disease temporally associated with the use of an investigational product, whether or not considered related to the study drug. Adverse events may also include pretreatment or posttreatment complications that occur as a result of protocol-specified procedures, or special situations.

An AE does not include the following:
Medical or surgical procedures such as surgery, endoscopy, tooth extraction, and transfusion.
Preexisting diseases, conditions, or laboratory abnormalities present or detected before the screening visit that do not worsen.
Situations where an untoward medical occurrence has not occurred (e.g., hospitalization for elective surgery, social and/or convenience admissions).
Overdose without clinical sequelae.
Any medical condition or clinically significant laboratory abnormality with an onset date before the consent form is signed and not related to a protocol-associated procedure is not an AE. Preexisting events or conditions that increase in severity or change in nature after the consent form is signed or as a consequence of participation in the clinical study will be considered AEs.

### Serious Adverse Events

A serious adverse event (SAE) is defined as an event that, at any dose, results in the following:
Death.
A life-threatening situation (the term “life-threatening” in the definition of “serious” refers to an event in which the participant was at risk of death at the time of the event; it does not refer to an event that hypothetically might have caused death if it were more severe).
Inpatient hospitalization or prolongation of existing hospitalization.
Persistent or significant disability/incapacity.
A congenital anomaly/birth defect.
A medically important event or reaction; such events may not be immediately life-threatening or result in death or hospitalization but may jeopardize the participant or may require intervention to prevent one of the other outcomes constituting SAEs. Medical and scientific judgment must be exercised to determine whether such an event is a reportable under expedited reporting rules. Examples of medically important events include intensive treatment in an emergency room or at home for allergic bronchospasm; blood dyscrasias or convulsions that do not result in hospitalization; and development of drug dependency or drug abuse.

### Preliminary Results

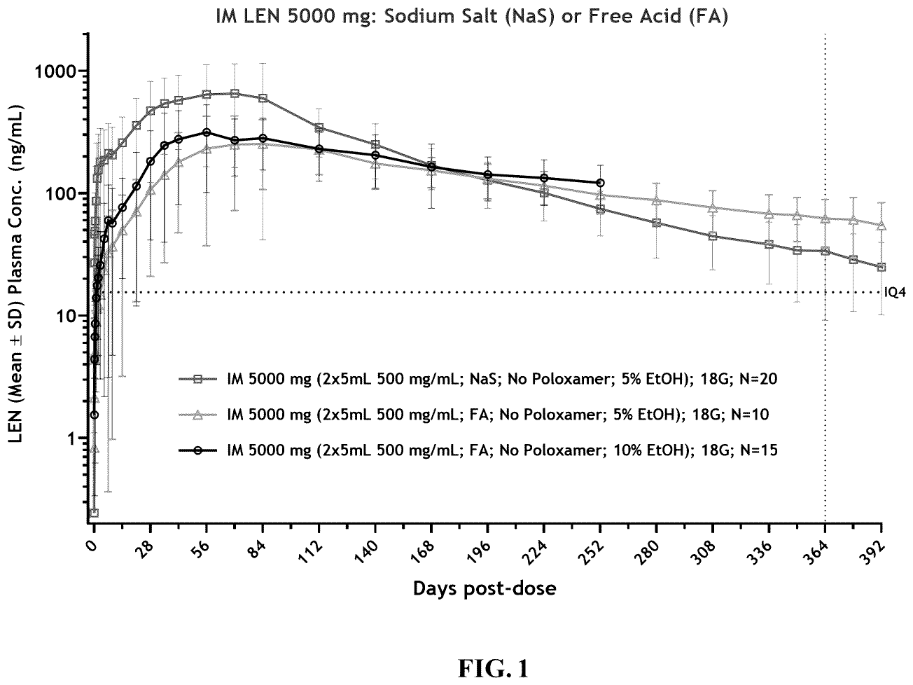

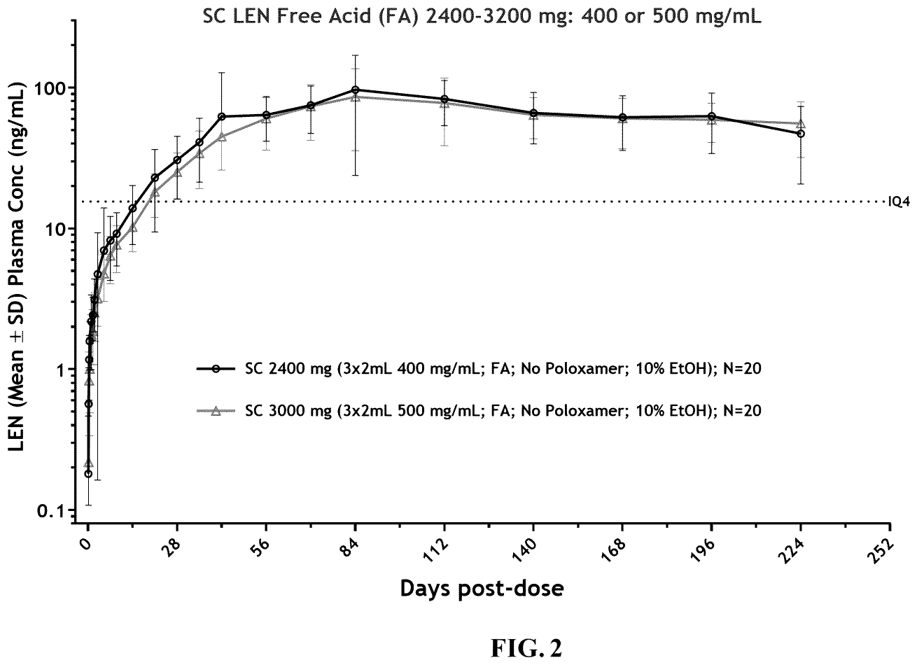

FIGS. 1-2 show preliminary results of LEN single doses in IM (FIG. 1) and SC (FIG. 2) studies. As shown in FIG. 1, a once-yearly intramuscular administration of Composition 2 (5000 mg lenacapavir sodium salt administered intramuscularly as two injections, 5 mL each at 500 mg/mL) or Composition 4 (5000 mg lenacapavir free acid administered intramuscularly as two injections, 5 mL each at 500 mg/mL, 5% ethanol in the formulation) maintained LEN mean plasma concentration in participants above inhibitory quotient-4 (IQ4; i.e., 4-fold in-vitro protein binding-adjusted 95% effective concentration) for at least 392 days post-dose. Further, a once-yearly intramuscular administration of Composition 3 (5000 mg lenacapavir free acid administered intramuscularly as two injections, 5 mL each at 500 mg/mL, 10% ethanol in the formulation) maintained LEN mean plasma concentration in patients above IQ4 for at least 252 days post-dose (based on the available data at the interim data-cut). An intramuscular administration of Composition 3 is particularly useful because the 10% ethanol included in the formulation reduces the viscosity and the injection time, improving the overall experience of the person receiving LEN.

As shown in FIG. 2, a once-yearly subcutaneous administration of Composition 6 (2400 mg LEN administered subcutaneously as three injections, 2 mL each at 400 mg/mL) or Composition 3 (3000 mg LEN administered subcutaneously as three injections, 2 mL each at 500 mg/mL) maintained mean plasma concentrations in patients above IQ4 for at least 224 days post-dose (based on the available data at the interim data-cut).

Mean trough concentrations of LEN>15.5 ng/mL, which is inhibitory quotient-4 (IQ4;), are associated with high rates of virologic suppression. LEN plasma concentrations at or above IQ4 are believed to provide near-maximal antiviral activity.

### Example 2. Pharmacokinetics and Safety of Once-Yearly Lenacapavir

This study evaluated the pharmacokinetics and safety of two once-yearly intramuscular lenacapavir formulations. In this phase 1 study of two intramuscular formulations of lenacapavir, we found pharmacokinetics adequate for once-yearly dosing and intramuscular administration to be safe and well tolerated. Mean plasma lenacapavir concentrations after intramuscular administration remained above the mean twice-yearly subcutaneous lenacapavir concentrations through 52 weeks, indicating that similarly high efficacy can be expected.

PURPOSE 1 and PURPOSE 2 were two phase 3, multicenter, double-blind, randomized, active-controlled trials in which twice-yearly subcutaneous lenacapavir (927 mg, administered as two 1.5 mL injections of 309 mg/mL, with oral loading doses of 600 mg on days 1 and 2) demonstrated superiority to daily oral PrEP, as well as a background incidence rate with 100% efficacy in cisgender women and 99.9% efficacy in cisgender men and gender-diverse people (see e.g., Bekker et al, N. Engl. J. Med. 2024; 391 (13): 1179-92; and Kelley et al, N. Engl. J. Med. 2024). In both studies, twice-yearly subcutaneous lenacapavir was well tolerated, with the most common adverse events being injection site reactions. Additionally, twice-yearly subcutaneous lenacapavir demonstrated better adherence than currently available daily oral PrEP options.

Twice-yearly lenacapavir has the potential to address multiple PrEP barriers because of its long duration of action and route of administration. For example, once-yearly intramuscular lenacapavir can build on this advantage by further decreasing the dosing frequency and need for clinic visits, reducing the potential for PrEP-related stigma, and avoiding the need for daily oral tablet adherence, thereby increasing the uptake of, persistence on, and, therefore, scalability and public health impact of PrEP in populations that would benefit most.

In this analysis, pharmacokinetics and safety of two different once-yearly intramuscular lenacapavir formulations were analyzed.

### Study Design and Participants

A multicenter, phase 1, open-label pharmacokinetics and safety study in healthy participants to evaluate lenacapavir formulations with once-yearly dosing potential was conducted. This example presents data from participants who were administered one of two lenacapavir formulations, as described below.

Eligible participants were healthy individuals assigned either male or female at birth and who were not pregnant or lactating, who were aged 18-55 years, with body-mass index≤35.0 kg/m², normal renal function (estimated creatinine clearance ≥90 mL/min by Cockcroft-Gault method), and no significant medical history. Participants had a low likelihood of HIV acquisition, as determined by the investigator at the screening evaluation. Participants tested negative for HIV with a laboratory instrumented fourth-generation HIV-1/2 antibody/antigen test at screening and with a point-of-care fourth-generation HIV-1/2 antibody/antigen test at day −1. Data on race and ethnicity were collected by participant self-report. Full inclusion and exclusion criteria were as follows:

Inclusion criteria
1) Have the ability to understand and sign a written informed consent form, which must be obtained before initiation of study procedures.
2) Be aged 18 through 55 years (inclusive) at screening.
3) Be a nonsmoker. The use of nicotine-containing products must be discontinued 42 days before the administration of study drug.
4) Have a calculated body-mass index not greater than 35.0 kg/m² at screening.
5) Have a creatinine clearance of at least 90 mL/min (using the Cockcroft-Gault method) based on serum creatinine and actual body weight, as measured at screening.
6) Participants who engage in heterosexual intercourse must agree to use contraception.
7) Participants have not donated blood within 56 days of study entry or plasma within 7 days of study entry, and must refrain from blood donation from clinic admission, throughout the study confinement period, and continuing through the duration of the study.
8) Screening laboratory evaluations and 12-lead electrocardiograms must be without clinically significant abnormalities as assessed by the investigator.
9) Have liver biometric test results of alanine aminotransferase, aspartate aminotransferase, alkaline phosphatase, and total bilirubin below or equal to the upper limit of normal at screening.
10) Must be willing and able to comply with all study requirements.
11) Must, in the opinion of the investigator, be in good health based upon medical history and physical examination, including vital signs.

Exclusion criteria
1) Have a positive serum or urine pregnancy test result.
2) Be a breastfeeding female.
3) Be currently participating in, or have participated in, an interventional clinical study with an investigational medicinal product within 30 days before study dosing on day 1 through the duration of the study.
4) Have a tattoo or other dermatological condition overlying the injection site that, in the opinion of the investigator, may interfere with interpretation of injection site reactions.
5) Have current alcohol or substance misuse judged by the investigator to potentially interfere with participant compliance or safety, or a positive drug or alcohol test result at screening or baseline.
6) Assessed by the investigator as being at risk of HIV infection in the past 6 months. This may include, but not be limited to, one or more of the following risk factors: (a) unprotected vaginal or anal sex with a person with HIV, (b) sex work for money or drugs, (c) sexually transmitted infection, (d) injection of non-prescribed drugs for recreational use, (e) post- or pre-exposure prophylaxis.
7) Have a positive test result for HIV at screening (antibody/antigen test) or day −1 (rapid test).
8) Have a positive test result for hepatitis B surface antigen or hepatitis C virus antibody at screening.
9) Have poor venous access that limits phlebotomy.
10) Have taken any prescription medications or over-the-counter medications, including herbal products, within 28 days before the start of study drug dosing, with the exception of vitamins and/or acetaminophen and/or ibuprofen and/or hormonal contraceptive medications.
11) Have been treated with systemic steroids, immunosuppressant therapies, or chemotherapeutic agents within 3 months before screening or is expected to receive these agents during the study (eg, corticosteroids, immunoglobulins, and other immune- or cytokine-based therapies).
12) Have a history of any of the following:
   a. Significant serious skin disease, such as (but not limited to) rash, food allergy, eczema, psoriasis, or urticaria.
   b. Significant drug sensitivity or drug allergy (such as anaphylaxis or hepatoxicity).
   c. Known hypersensitivity to the study drug, its metabolites, or to formulation excipients.
   d. Significant cardiac disease (including history of myocardial infarction based on ECG and/or clinical history, any history of ventricular tachycardia, congestive heart failure, or dilated cardiomyopathy with left ventricular ejection fraction <40%), a family history of long QT interval syndrome, or unexplained death in an otherwise healthy individual between the ages of 1 and 30 years.
   e. Syncope, palpitations, or unexplained dizziness.
   f. Implanted defibrillator or pacemaker.
   g. Liver disease, including Gilbert syndrome.
   h. Severe peptic ulcer disease, gastroesophageal reflux disease, or other gastric acid hypersecretory conditions requiring prolonged (>6 months) medical treatment.
   i. Have any serious or active medical or psychiatric illness (including depression) that, in the opinion of the investigator, would interfere with participant treatment, assessment, or compliance with the protocol. This would include renal, cardiac, haematological, hepatic, pulmonary (including chronic asthma), endocrine (including diabetes), central nervous, gastrointestinal (including an ulcer), vascular, metabolic (thyroid disorders, adrenal disease), and immunodeficiency disorders; active infection; or malignancy that are clinically significant or requiring treatment.

### Procedures

Participants meeting the eligibility criteria were enrolled and received a single dose of 5000 mg lenacapavir, administered as 2×5 mL intramuscular injections of either Composition 4 or Composition 3 (see Example 1, Table 1). Each formulation contained 500 mg/mL lenacapavir free acid, formulated with 5% w/w ethanol (Composition 4) or 10% w/w ethanol (Composition 3), included to reduce viscosity. Intramuscular doses were injected into the ventrogluteal site on day 1. Half of the participants who received Composition 3 were pretreated for approximately 10 minutes with an ice pack at the sites of injection.

Participants remained at the study center for observation and assessment from day −1 through day 15. Intensive plasma pharmacokinetic sampling occurred relative to the morning dose of lenacapavir at the following time points for each cohort: day 1 at 0 (predose; ≤5 minutes before dose), 2, 4, 8, and 12 hours post-dose; day 2 at 24 and 36 hours post-dose. Following intensive pharmacokinetic sampling, A single anytime pharmacokinetic sample was collected on days 3, 4, 6, 8, 10, 15, 22, 29, 36, 43, 57, 71, 85, 113, 141, 169, 197, 225, 253, 281, 309, 337, 351, 365, 379, and 393; and at the early termination visit (if applicable). Additional single anytime pharmacokinetic samples will be collected on days 421, 449 for both formulations (and days 477, 505, 533, 561, and 603 for Composition 3).

Safety assessments included a complete physical examination at screening and before discharge, and a symptom-driven physical examination at each visit based on reported signs and symptoms. Vital signs and laboratory assessments, including HIV-1/2 antibody/antigen testing, occurred at screening and at prespecified time points throughout the study. Injection sites were examined on day 1 (3 hours after study drug injection), daily on day 2 through day 15 (before discharge), and at each outpatient visit based on reported signs and symptoms. Participants completed a pain questionnaire on day 1 (following injection), day 2 (24 hours after study drug injection), day 7, and day 15 (before discharge). Assessment of adverse events and concomitant medications was performed throughout the study. All adverse events and clinically significant laboratory abnormalities were graded according to the Division of AIDS (DAIDS) Table for Grading the Severity of Adult and Pediatric Adverse Events, Version 2.1 (July 2017).

### Outcomes

Pharmacokinetic parameters evaluated included the area under the concentration-time curve for the once-yearly dosing interval calculated from days 1-365 (AUC_{days 1-365}), mean peak plasma concentration (Cₘₐₓ), mean time to reach peak plasma concentration (Tₘₐₓ), and mean trough concentration (C_{trough}) at the end of 52 weeks (day 365). Safety outcomes included the results of the safety assessments outlined above.

Mean lenacapavir concentrations of once-yearly intramuscular formulations were compared to those observed in phase 3 studies of twice-yearly subcutaneous lenacapavir for PrEP, which were established by pooling the observed concentrations from PURPOSE 1 and 2 (see e.g., Bekker et al, N. Engl. J. Med. 2024; 391 (13): 1179-92; and Kelley et al, N. Engl. J. Med. 2024). To ensure trough concentrations remained similar to those observed in PURPOSE 1 and 2, the median C_{trough} at 52 weeks following once-yearly intramuscular lenacapavir administration was compared with the median twice-yearly subcutaneous lenacapavir C_{trough} at 26 weeks.

### Bioanalysis of Plasma Lenacapavir Concentrations

Plasma concentrations of lenacapavir were determined using a validated liquid chromatography-tandem mass spectroscopy bioanalytical method with multiple reaction monitoring and electrospray ionization in the positive mode (Labcorp, Madison, WI, USA). Quantification was performed using multiple reaction monitoring of the transitions m/z 968.4→869.3 and m/z 974.4→875.3 for lenacapavir and an isotopically labelled internal standard (lenacapavir-d6), respectively. The bioanalytical method was validated over a calibrated range of 0.5-500 ng/mL. Interassay precision, based on coefficient of variation (% CV), ranged from 2.8 to 8.5, and accuracy (% relative error) ranged from −6.5 to −4.6. All samples were analyzed in the timeframe supported by frozen stability storage data.

### Statistical Analysis

Noncompartmental analyses using Phoenix WinNonlin Professional software (version 8.2; Certara, Princeton, NJ, USA) were performed to estimate the pharmacokinetic parameters of lenacapavir. Lenacapavir pharmacokinetic parameters were summarized using descriptive statistics. Treatment-emergent adverse events, including injection site reactions and laboratory abnormalities, were descriptively summarized. The safety analyses were conducted using SAS (version 9.4, SAS Institute).

### Results

A total of 20 participants received lenacapavir Composition 4 (lenacapavir 500 mg/mL with 5% w/w ethanol) and 20 participants received lenacapavir Composition 3 (lenacapavir 500 mg/mL with 10% w/w ethanol) as a single 5000 mg dose administered by intramuscular injection (2×5 mL). Participant demographics and baseline characteristics are shown in Table 2. Data presented in Table 2 are n (%) or median (Q1, Q3).

TABLE 2
Demographics and Baseline Characteristics

| | Lenacapavir | | Lenacapavir | |
|---|---|---|---|---|
| | Composition 4 | | Composition 3 | |
| | (N = 20) | | (N = 20) | |
| Age, years | 37 | (29, 50) | 33 | (29, 45) |
| Assigned male sex at birth | 13 | (65%) | 13 | (65%) |
| Assigned female sex at birth | 7 | (35%) | 7 | (35%) |
| Race (self-reported) | | | | |
| Black or African American | 3 | (15%) | 5 | (25%) |
| White | 17 | (85%) | 15 | (75%) |
| Ethnicity (self-reported) | | | | |
| Hispanic or Latine | 20 | (100%) | 16 | (80%) |
| Not Hispanic or Latino | 0 | | 4 | (20%) |
| Weight, kg | 73.6 | (68.6, 86.8) | 77.1 | (72.5, 85.6) |
| Body-mass index, kg/m² | 26.5 | (24.1, 29.4) | 28.0 | (24.9, 30.0) |

The median age was 37 years in the Composition 4 cohort and 33 years in the Composition 3 cohort; 13/20 (65%) participants in each cohort were assigned male at birth. Of those who received Composition 4, 17/20 (85%) were White and 20/20 (100%) were of Hispanic or Latine ethnicity. Of those who received Composition 3, 15/20 (75%) were White and 16/20 (80%) were of Hispanic or Latine ethnicity. The median (Q1, Q3) body weight for participants who received Composition 4 was 73.6 (68.6, 86.8) kg and for participants who received Composition 3, 77.1 (72.5, 85.6) kg. For the pharmacokinetic analysis, there were a total of 13 participants for Composition 4 and 19 participants for Composition 3 at the end of week 52 weeks due to early discontinuations for reasons unrelated to the study drug or missing pharmacokinetic data leading up to the data cut-off.

Pharmacokinetic parameters of lenacapavir following administration of both formulations are presented in Table 3, where % CV=percentage coefficient of variation; AUC=area under the concentration-time curve; AUC_{days 1-365}=area under the concentration-time curve for the once-yearly dosing interval calculated from days 1-365; Cₘₐₓ=peak plasma concentration; C_{trough} (day 365)=trough concentration at day 365; Q1=first quartile; Q3=third quartile; and Tₘₐₓ=time to reach peak plasma concentration. Pharmacokinetic parameters Cₘₐₓ, AUC_{days 1-365}, and C_{trough} (day 365) are presented as mean (% CV), Tₘₐₓ is presented as median (Q1, Q3). For Composition 4, n=15 (Cₘₐₓ and Tₘₐₓ) and n=13 (AUC_{days 1-365} and C_{trough}); for Composition 3, n=19 (AUC_{days 1-365} and C_{trough}).

TABLE 3
Plasma pharmacokinetic parameters following once-yearly
intramuscular administration of 5000 mg lenacapavir

| | Composition 4: | | Composition 3: | |
|---|---|---|---|---|
| | Lenacapavir 5000 mg | | Lenacapavir 5000 mg | |
| | (2 × 5 mL 500 mg/mL | | (2 × 5 mL 500 mg/mL | |
| | with 5% w/w ethanol) | | with 10% w/w ethanol) | |
| | (N = 20) | | (N = 20) | |
| Cₘₐₓ, ng/mL | 294 | (62.2) | 376 | (53.0) |
| Tₘₐₓ, days | 84.1 | (56.1, 112) | 69.9 | (55.3, 105) |
| AUC_{days 1-365}, | 1153 | (38.0) | 1368 | (35.7) |
| h*μg/mL | | | | |
| C_{trough (day 365)}, | 62.2 | (42.8) | 73.6 | (47.9) |
| ng/mL | | | | |

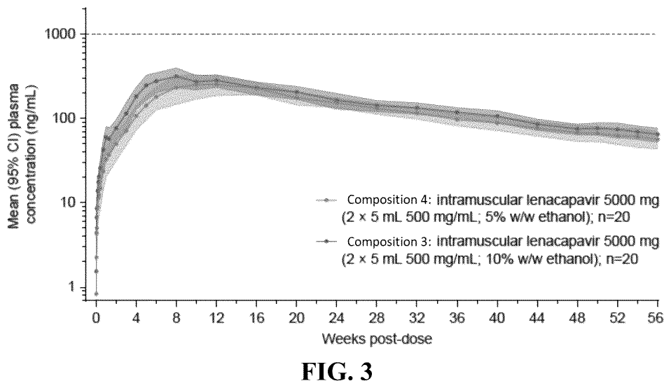

Following intramuscular administration of Composition 4 and Composition 3, plasma concentrations increased rapidly, and median Tₘₐₓ occurring approximately 12 weeks post-dose for Composition 4 and approximately 10 weeks post-dose for Composition 3. Mean lenacapavir Cₘₐₓ (% CV) was 294 (62.2) ng/ml with Composition 4 and 376 (53.0) ng/ml with Composition 3, as shown in Table 3 and FIG. 3.

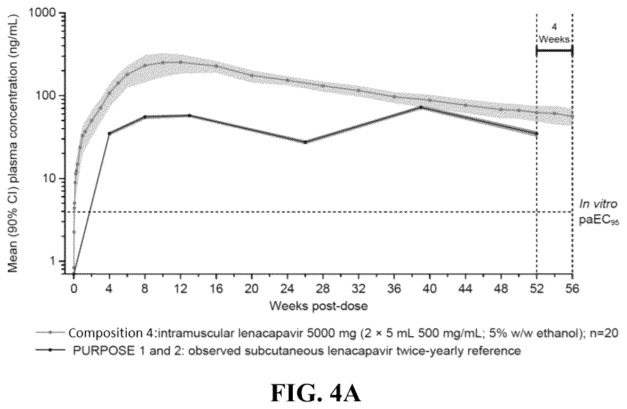

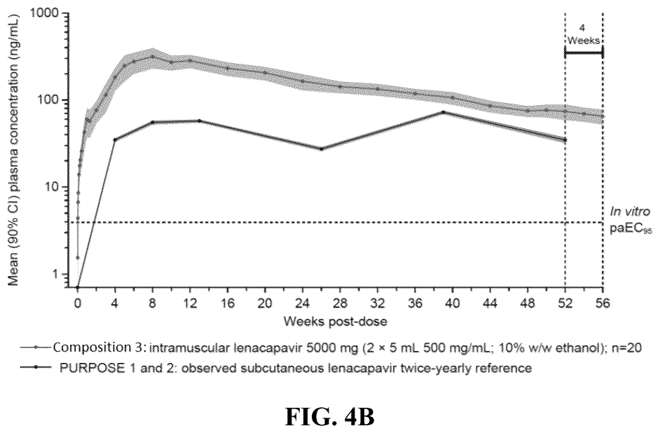

When evaluating the similarity of once-yearly intramuscular formulations to the twice-yearly subcutaneous formulation, mean lenacapavir concentrations with once-yearly intramuscular lenacapavir remained higher than the mean concentrations with twice-yearly subcutaneous lenacapavir observed in PURPOSE 1 and 2 through at least 56 weeks, as shown in FIGS. 4A-4B.

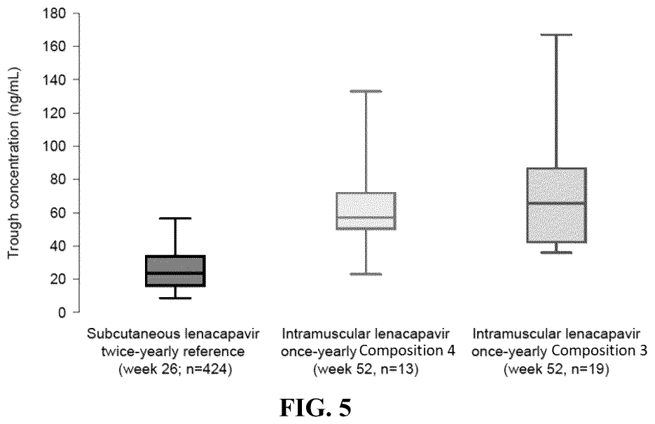

The observed median lenacapavir C_{trough} at the end of 52 and 56 weeks for participants who received Composition 4 (n=13) were 57.0 ng/ml and 50.7 ng/ml, respectively, and for those who received Composition 3 (n=19), 65.6 ng/ml and 55.9 ng/mL, respectively, in comparison with the median observed C_{trough} of 23.4 ng/ml in PURPOSE 1 and 2 at week 26, as shown in FIG. 5. The lenacapavir C_{trough} at the end of 52 weeks for all participants following Composition 4 and Composition 3 were similar to or higher than the lenacapavir C_{trough} for twice-yearly subcutaneous lenacapavir at the end of 26 weeks.

Treatment-emergent adverse events and grade ≥3 laboratory abnormalities are shown in Table 4. Data presented in Table 4 are n (%). Gait disturbance was defined as difficulty walking due to pain at the injection site but did not appear to limit daily activities.

TABLE 4
Treatment-Emergent Adverse Events (TEAEs)

| | Lenacapavir | Lenacapavir |
|---|---|---|
| | Composition 4 | Composition 3 |
| | (N = 20) | (N = 20) |
| Any TEAE | 18 (90%) | 16 (80%) |
| Adverse events occurring in ≥10% | | |
| of participants in a cohort | | |
| Diarrhoea | 2 (10%) | 0 |
| Injection site pain | 16 (80%) | 15 (75%) |
| Injection site bruising | 2 (10%) | 1 (5%) |
| Injection site swelling | 4 (20%) | 0 |
| Gait disturbance | 0 | 4 (20%) |
| Feeling hot | 0 | 2 (10%) |
| Headache | 0 | 5 (25%) |
| Dizziness | 0 | 2 (10%) |
| Study drug-related TEAEs | 17 (85%) | 16 (80%) |
| Any grade ≥3 TEAEs | 0 | 2 (10%) |
| Study drug-related grade ≥3 TEAEs | 0 | 1 (5%)† |
| Any serious TEAEs | 0 | 1 (5%) |
| Study drug-related serious TEAES | 0 | 0 |
| Death | 0 | 0 |
| Grade ≥3 laboratory abnormalities | 6 (30%)‡ | 3 (15%)§ |

†There was one participant with lenacapavir-related grade 3 injection site pain and syncope who received Composition 3.
‡n = 3 increased low-density lipoprotein; n = 1 each of increased creatinine kinase, increased lipase, hyperkalaemia, increased triglycerides, glycosuria.
§n = 2 decreased creatinine clearance; n = 1 hypercholesterolemia; n = 1 increased low-density lipoprotein.

Most treatment-emergent adverse events were mild or moderate. There were no grade 4 treatment-emergent adverse events, laboratory abnormalities, or deaths. Among participants who received Composition 3, 4/20 (20%) experienced an adverse event of gait disturbance, which was defined as difficulty walking due to pain at the injection site, and this did not appear to interfere with daily activities per the investigator's report. None of these four participants received pretreatment with ice.

Most injection site reactions experienced by participants were grade 1 or 2. Injection site pain was reported by 16/20 (80%) participants who received Composition 4 and 15/20 (75%) who received Composition 3. Injection site bruising occurred in 2/20 (10%) participants receiving Composition 4 and 1/20 (5%) receiving Composition 3. Injection site swelling occurred in 4/20 (20%) participants receiving Composition 4, but 0/20 participants receiving Composition 3. Longer-lasting injection site reactions, such as nodules, were not observed with either formulation. The median (Q1, Q3) duration of any lenacapavir-related injection site reaction was 4 (2, 5) days following Composition 4 and 3 (3, 4) days following Composition 3.

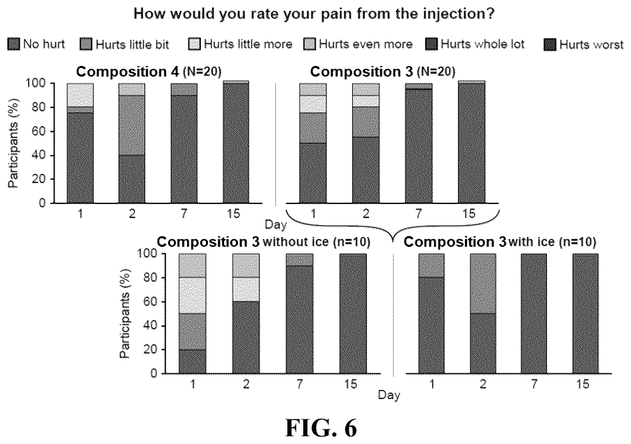

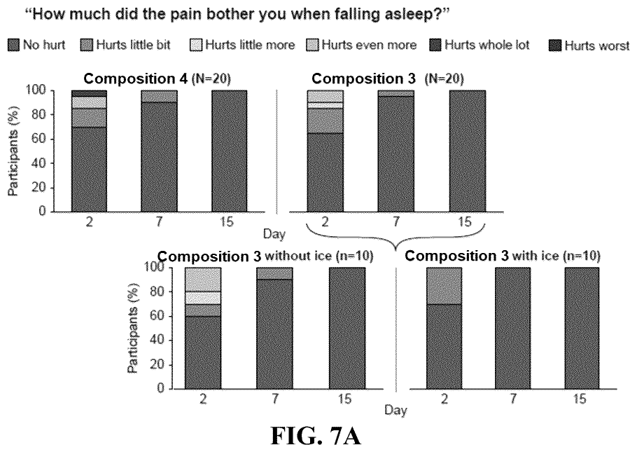

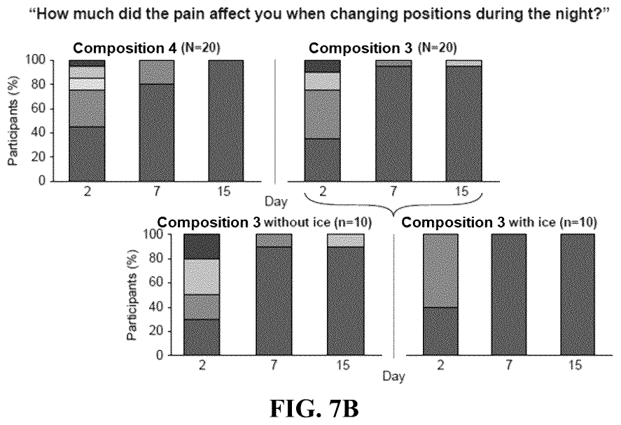

Participant-reported outcomes that were collected via participant questionnaire are shown in FIG. 6. Most participants reported minor pain on days 1 and 2 only and none chose the options “Hurts whole lot” or “Hurts worst.” Ice pretreatment (n=10) resulted in numerically lower pain ratings on days 1 and 2 for Composition 3, with the majority resolved by day 7. Additional questions were asked regarding the impact of injection site pain on sleep. Responses were similar, as shown in FIG. 7A-7B.

### Discussion

In this study of two lenacapavir formulations administered intramuscularly, plasma lenacapavir concentrations remained above those associated with twice-yearly subcutaneous lenacapavir efficacy for more than one year post-administration. Injections were generally well tolerated, with injection site pain being the most common adverse event. No clinically significant safety concerns were identified.

The mean lenacapavir concentrations observed with once-yearly intramuscular lenacapavir formulations exceeded those associated with high efficacy in PURPOSE 1 and 2 (see e.g., Bekker et al, N. Engl. J. Med. 2024; 391 (13): 1179-92; and Kelley et al, N. Engl. J. Med. 2024). For both formulations, median C_{trough} after 52 weeks post-administration was higher than the median C_{trough} at 26 weeks observed in PURPOSE 1 and 2. These findings suggest that once-yearly intramuscular lenacapavir should confer similar HIV prevention efficacy as twice-yearly subcutaneous lenacapavir, thereby creating the potential to expedite the development of once-yearly lenacapavir by allowing for the extrapolation of efficacy from twice-yearly subcutaneous lenacapavir to once-yearly intramuscular lenacapavir. Use of model-informed drug development approaches to extrapolate efficacy between drug formulations or routes of administration have been used, for example, in development of antipsychotics, cancer therapeutics, and autoimmune treatments (see e.g., Samtani et al, CNS Drugs 2011; 25 (10): 829-45; Hard et al, J Clin Psychopharmacol 2017; 37 (3): 289-95; Morrissey et al, Cancer Chemother Pharmacol 2019; 84 (6): 1257-67; Masters et al, CPT Pharmacometrics Syst Pharmacol 2022; 11 (4): 458-68; Liu et al, Lupus 2022; 31 (4): 424-32; Pisal et al, Clin Pharmacol Ther 2025; 117 (2): 475-84; Yenerel et al, Adv Ther 2023; 40 (1): 211-232; and Wang et al, Clin Pharmacol Ther 2024; 115 (3): 440-451). For example, with the monoclonal antibody, ravulizumab, clinical pharmacokinetic data showing comparability to the intravenous dosing regimen with previously established efficacy and safety was used for the approval of the subcutaneous route of administration (see e.g., Yenerel et al, Adv Ther 2023; 40 (1): 211-232).

The highest observed mean concentrations with the once-yearly formulations (294 ng/ml for Composition 4; 376 ng/mL for Composition 3) also exceeded the highest observed mean concentration with twice-yearly subcutaneous lenacapavir (72.1 ng/ml) (see e.g., Jogiraju et al, Pharmacokinetics of a Simplified Subcutaneous Lenacapavir Regimen Versus Phase 2/3 Regimen. Poster presentation #PESUB22 at AIDS 2022, 29 Jul.-2 Aug.). This observation is attributable to the higher total dose needed to achieve once-yearly pharmacokinetics and the more rapid drug absorption observed with intramuscular administration. However, systemic lenacapavir exposures up to approximately 3-fold higher than those achieved with these intramuscular lenacapavir formulations have been observed previously and were found to be well tolerated with no safety concerns, as shown in FIG. 3 (see e.g., Jogiraju et al, Antimicrob Agents Chemother 2024; 68 (4): e0134423; and Sunlenca (lenacapavir) [Package Insert]). These data indicate that the therapeutic window of lenacapavir is wide and support further evaluation of once-yearly intramuscular lenacapavir for PrEP.

Injection tolerability is an important consideration in the development of long-acting injectables. Twice-yearly subcutaneous lenacapavir was well tolerated in phase 3 studies; palpable nodules occurring due to the subcutaneous lenacapavir drug depot were observed in some participants (see e.g., Castagna et al, Follow-Up of Injection Site Reactions in Clinical Studies of People Using Lenacapavir Every 6 Months for HIV Treatment [abstract eP.A.104]. In: Program and abstracts of the 19th European AIDS Conference (EACS) (Warsaw, Poland); 2023; and Kumar et al, Injection-Site Reaction Experience in Clinical Studies of People Using Lenacapavir For HIV Treatment [abstract EPB184]. In: Program and abstracts of the 24th International AIDS Conference (Montreal, Canada); 2022). Despite the high volume of injection, intramuscular lenacapavir was well tolerated in this study. Injection-related pain was commonly reported but was of mild to moderate severity in most participants. Pre-treatment of the injection site with an ice pack appeared to substantially reduce injection pain. Superficial injection site reactions were uncommon, with only isolated reports of bruising or swelling. These findings suggest that once-yearly intramuscular lenacapavir could be a well-tolerated PrEP option, and the availability of an intramuscular route of administration for lenacapavir in addition to a twice-yearly subcutaneous formulation could provide another choice for people taking PrEP and allow greater individualization of care.

As the doses administered in this study resulted in lenacapavir concentrations higher than those observed with twice-yearly subcutaneous lenacapavir, the optimal dose needed to match the relevant pharmacokinetic parameters from twice-yearly subcutaneous lenacapavir is likely to be lower than 5000 mg. Oral lenacapavir loading doses were not given with intramuscular lenacapavir in this study, in contrast to twice-yearly subcutaneous lenacapavir, which requires oral lenacapavir to be administered on days 1 and 2 at the time of the first injection administration because of the slow initial release of lenacapavir from the subcutaneous drug depot. Here, a faster increase in initial lenacapavir plasma concentration was observed with the intramuscular lenacapavir formulations than with the twice-yearly subcutaneous formulation. However, the intramuscular formulations still took a few days to reach concentrations similar to the PURPOSE 1 and 2 studies, and therefore oral loading may be needed (see e.g., Jogiraju et al, Pharmacokinetics of a Simplified Subcutaneous Lenacapavir Regimen Versus Phase 2/3 Regimen. Poster presentation #PESUB22 at AIDS 2022, 29 Jul.-2 Aug.). The total dose and oral loading regimen for a once-yearly intramuscular lenacapavir to achieve similar pharmacokinetics to twice-yearly subcutaneous lenacapavir will be determined using population pharmacokinetic modelling and simulation.

### Summary

Two intramuscular lenacapavir formulations maintained plasma concentrations higher than those associated with HIV prevention efficacy in phase 3 studies of twice-yearly subcutaneous lenacapavir for the duration of a year, indicating that intramuscular lenacapavir could provide similarly high preventative efficacy to twice-yearly subcutaneous lenacapavir but with an annual dosing interval. Both formulations of intramuscular lenacapavir were well tolerated and no unexpected safety findings occurred. These data support the planned development of once-yearly intramuscular lenacapavir for HIV PrEP, which has the potential to improve PrEP uptake and persistence.

### Example 3. Synthesis of Lenacapavir Free Acid

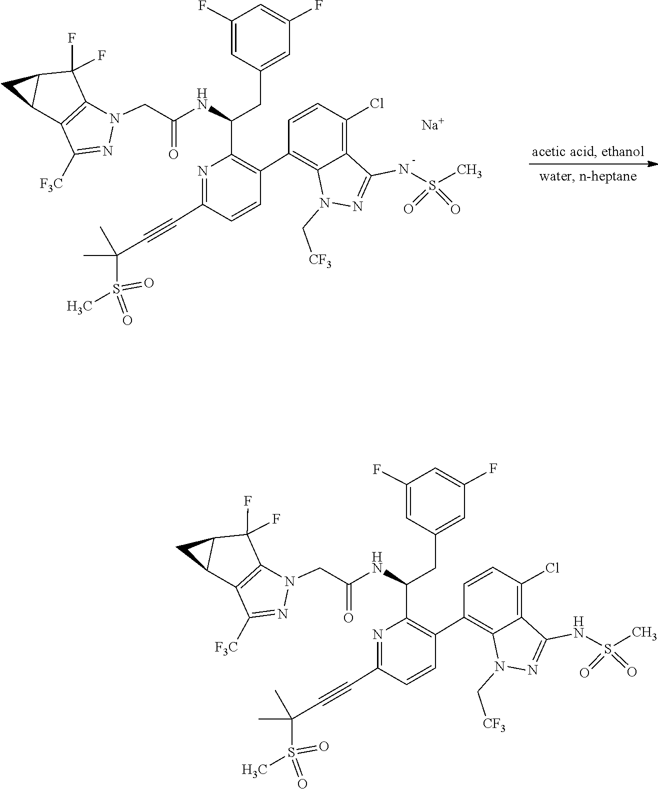 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O ; [Na+] ; CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Lenacapavir sodium (45 g, 1.0 equivalent), anhydrous ethanol (185 g), and glacial acetic acid (13.5 g) were charged to a reactor. The temperature was adjusted, and the mixture was agitated until a clear solution was achieved. The organic solution was polish filtered, slowly transferred to high purity water (900 g) in another reactor, and rinsed forward with anhydrous ethanol (22.5 g). The resulting slurry was agitated and solids were isolated by filtration. The filter cake slurry was washed with high purity water (450 g), followed by additional rinses with high purity water (225 g) and n-heptane (315 g). The filter cake was de-liquored and dried under vacuum to afford 40 g (92%) lenacapavir free acid. 1H NMR (400 MHz, DMSO d₆): δ 0.96 (m, 1H), 1.40 (m, 1H), 1.74 (s, 6H), 2.55 (m, 1H), 2.60 (m, 1H), 2.95 and 2.92 (dd, m, 1H), 3.02 and 3.11 (dd, dd, 1H), 3.17 and 3.24 (s, s, 3H), 3.27 (s, 3H), 3.96 and 4.19 (dq, dq, 1H), 4.51 and 4.68 (dq, m, 1H), 4.71 and 4.60 (d, m, 1H), 9.25 and 8.92 (d, d, 1H), 4.59 and 4.74 (m, m, 1H), 4.92 and 4.77 (d, d, 1H), 6.48 and 6.54 (m, m, 2H), 7.02 and 6.94 (tt, m, 1H), 6.86 and 7.19 (d, d, 1H), 7.32 (d, 1H); 7.76 and 7.79 (d, m, 1H), 7.81 and 7.90 (d, d, 1H), 10.00 and 10.11 (s, s, 1H).

Various modifications of the invention, in addition to those described herein, will be apparent to those skilled in the art from the foregoing description. Such modifications are also intended to fall within the scope of the appended claims. Each reference, including all patent, patent applications, and publications, cited in the present disclosure is incorporated herein by reference in its entirety.

1. A pharmaceutical composition comprising a free acid form of the compound of Formula Ia: 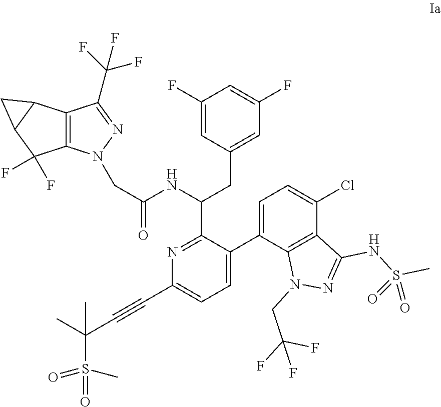 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
PEG 300, ethanol, and water, wherein the composition comprises:
about 30% to about 45% of the free acid form of the compound of Formula Ia;
about 37% to about 50% of the PEG 300;
about 7% to about 15% of the ethanol; and
about 5% to about 10% of the water.

2. The pharmaceutical composition of claim 1, wherein the composition comprises:
about 30% to about 45% of the free acid form of the compound of Formula Ia;
about 40% to about 50% of the PEG 300;
about 7% to about 15% of the ethanol; and
about 5% to about 10% of the water.

3. The pharmaceutical composition of claim 1, wherein the composition comprises about 40% to about 43% of the free acid form of the compound of Formula Ia.

4. The pharmaceutical composition of claim 1, wherein the composition comprises about 42% to about 43% of the free acid form of the compound of Formula Ia.

5. The pharmaceutical composition of claim 1, wherein the composition comprises about 42% of the free acid form of the compound of Formula Ia.

6. The pharmaceutical composition of claim 1, wherein the composition comprises about 42.02% of the free acid form of the compound of Formula Ia.

7. The pharmaceutical composition of claim 1, wherein the composition comprises about 40% to about 45% of the PEG 300.

8. The pharmaceutical composition of claim 1, wherein the composition comprises about 40% to about 41% of the PEG 300.

9. The pharmaceutical composition of claim 1, wherein the composition comprises about 41% of the PEG 300.

10. The pharmaceutical composition of claim 1, wherein the composition comprises about 40.78% of the PEG 300.

11. The pharmaceutical composition of claim 1, wherein the composition comprises about 6% to about 9% of the water.

12. The pharmaceutical composition of claim 1, wherein the composition comprises about 7% to about 8% of the water.

13. The pharmaceutical composition of claim 1, wherein the composition comprises about 7% of the water.

14. The pharmaceutical composition of claim 1, wherein the composition comprises about 7.20% of the water.

15-26. (canceled)

27. The pharmaceutical composition of claim 1, wherein the composition comprises about 9% to about 11% of the ethanol.

28. The pharmaceutical composition of claim 1, wherein the composition comprises about 10% of the ethanol.

29. The pharmaceutical composition of claim 1, wherein the composition consists of:
about 30% to about 45% of the free acid form of the compound of Formula Ia;
about 40% to about 50% of the PEG 300;
about 7% to about 15% of the ethanol; and
about 5% to about 10% of the water.

30. The pharmaceutical composition of claim 1, wherein the composition comprises:
about 40% to about 43% of the free acid form of the compound of Formula Ia;
about 40% to about 42% of the PEG 300;
about 9% to about 11% of the ethanol; and
about 6% to about 8% of the water.

31. (canceled)

32. The pharmaceutical composition of claim 1, wherein the composition comprises:
about 41% to about 43% of the free acid form of the compound of Formula Ia;
about 37% to about 43% of PEG 300;
about 9% to about 11% of ethanol; and
about 6% to about 8% of water.

33. The pharmaceutical composition of claim 1, wherein the composition comprises:
about 42% of the free acid form of the compound of Formula Ia;
about 37% to about 43% of PEG 300;
about 9% to about 11% of ethanol; and
about 6% to about 8% of water.

34. The pharmaceutical composition of claim 1, wherein the composition comprises:
about 42.02% of the free acid form of the compound of Formula Ia;
about 37% to about 43% of PEG 300;
about 9% to about 11% of ethanol; and
about 6% to about 8% of water.

35. The pharmaceutical composition of claim 1, wherein the composition comprises:
about 42% to about 43% of the free acid form of the compound of Formula Ia;
about 40% to about 41% of the PEG 300;
about 9% to about 11% of the ethanol; and
about 7% to about 8% of the water.

36. The pharmaceutical composition of claim 1, wherein the composition comprises:
about 42.02% of the free acid form of the compound of Formula Ia;
about 40.78% of the PEG 300;
about 10% of the ethanol; and
about 7.20% of the water.

37-40. (canceled)

41. The pharmaceutical composition of claim 1, wherein the composition comprises about 300 mg/mL to about 600 mg/mL of the free acid form of the compound of Formula Ia.

42. The pharmaceutical composition of claim 1, wherein the composition comprises about 400 mg/mL to about 500 mg/mL of the free acid form of the compound of Formula Ia.

43. The pharmaceutical composition of claim 1, wherein the composition comprises about 500 mg/mL of the free acid form of the compound of Formula Ia.

44. (canceled)

45. The pharmaceutical composition of claim 1, wherein the composition is a solution.

46-47. (canceled)

48. The pharmaceutical composition of claim 1, wherein the composition does not comprise a poloxamer.

49. The pharmaceutical composition of claim 1, wherein the free acid form of the compound of Formula Ia is a free acid form of the compound of Formula Ib:

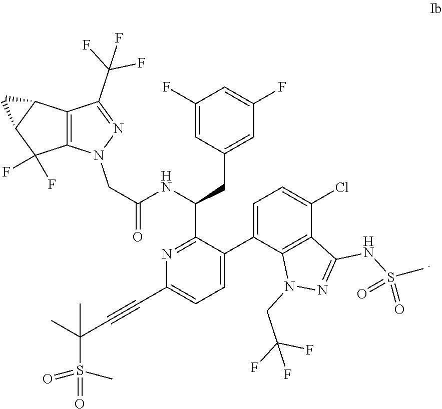 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

50. A method of treating or preventing HIV in a patient, comprising administering to the patient a pharmaceutical composition of claim 1.

51-71. (canceled)

72. A method of reducing the risk of sexually acquired HIV in a patient, comprising administering to the patient a composition of claim 1.

73. (canceled)

74. A method of reducing the risk of sexually acquired HIV in a patient, comprising administering to the patient a compound of Formula Ia: 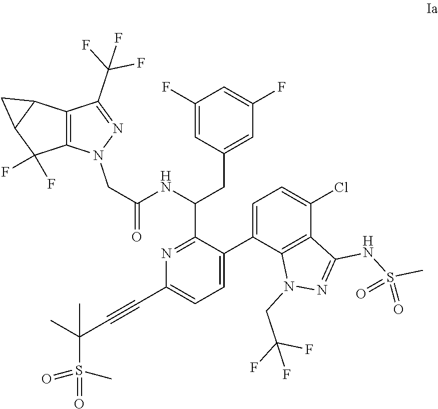 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
or a pharmaceutically acceptable salt thereof, the method comprising:
(i) administering to the patient an initiation dosage comprising:
intramuscularly administering to the patient about 3000 mg of the compound of Formula Ia, or a pharmaceutically acceptable salt thereof, on the first day;
orally administering to the patient about 600 mg of the compound of Formula Ia, or a pharmaceutically acceptable salt thereof, on the first day; and
orally administering to the patient about 600 mg of the compound of Formula Ia, or a pharmaceutically acceptable salt thereof, on the second day; and
(ii) intramuscularly administering to the patient a pharmaceutical composition comprising a free acid form of the compound of Formula Ia, PEG 300, ethanol, and water, once every twelve months from the first day, wherein the composition comprises:
about 30% to about 45% of the free acid form of the compound of Formula Ia;
about 37% to about 50% of the PEG 300;
about 7% to about 15% of the ethanol; and
about 5% to about 10% of the water.

75-92. (canceled)
